(19) United States
(12) Patent Application Publication
(10) Pub. No.: US 2003/0082598 A1
(43) Pub. Date: May 1, 2003

(54) Molecular interaction sites of 23S ribosomal RNA and methods of modulating the same

(76) Inventor: David J. Ecker, Encinitas, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/224,836

(22) Filed: Aug. 20, 2002

Related U.S. Application Data
(60) Provisional application No. 60/314,251, filed on Aug. 22, 2001.

(51) Int. Cl. C12Q 1/68; C07H 21/04

(52) U.S. Cl. 435/6; 536/23.1

(57) ABSTRACT

Polynucleotides comprising molecular interaction sites of 23S rRNA that have particular secondary structure are provided. Methods of using such polynucleotides to screen, virtually or actually, combinatorial libraries of compounds that bind thereto are also provided. Method of modulating the activity of 23S rRNA by contacting 23S rRNA or prokaryotic cells containing the same with a compound identified by such virtual or actual screening are also provided.

### CROSS-REFERENCE TO RELATED APPLICATIONS

This application claims priority to U.S. provisional application Serial No. 60/314,251 filed Aug. 22, 2001, which is incorporated herein by reference in its entirety.

### FIELD OF THE INVENTION

The present invention relates to identification of molecular interaction sites of 23S rRNA, virtual or actual screening of compounds that bind thereto, and to modulating the activity of 23S rRNA with such compounds identified in the actual or virtual screening.

### BACKGROUND OF THE INVENTION

Ribosomes are large, multisubunit ribonucleoprotein complexes (RNPs) that are responsible for protein synthesis, and are highly conserved, both structurally and functionally, across microbial phyla. They include large (50S) and small (30S) subunits that are assembled from ribosomal RNAs (rRNAs) and proteins bound to the rRNA. The 50S ribosomal subunit contains the 23S rRNA. Ribosomes synthesize proteins when correctly bound to messenger RNA (rnRNA) and transfer RNA (tRNA). It is now generally accepted that the sites of action of numerous antimicrobial compounds that inhibit ribosomes lie within 23S rRNA. Very large molecules such as ribosomes are not, however, usually desirable targets for high-throughput screens.

Several factors related to the structural complexity of the ribosome complicate screening assays that rely on binding of a potential drug candidate to a ribosomal target, including difficulty in obtaining large quantities of purified ribosomes and degradation of ribosomes under typical screening conditions.

It is now generally accepted that 16S and 23S rRNAs play important, if not critical, roles in the decoding and peptidyl transferase activities of ribosomes. A major target of anti-bacterial antibiotics are the catalytic rRNAs of prokaryotic ribosomes. Prokaryotes posses a 50S subunit that houses 23S and 5S rRNA, and a 30S ribosome subunit that houses 16S rRNA. The 50S ribosome contains the peptidyl-transferase and GTPase activities. The 3′ site acceptor end of tRNA interacts with a conserved region in the 23S rRNA. Specific nucleotides in 23S rRNA are targeted by numerous MLS compounds (macrolides, lincomycins, and streptogramins), including erythromycin.

Erythromycin binds to the 23S rRNA and inhibits translation. Tetracycline binds to the 23S rRNA subunit and inhibits binding of aminoacyl-tRNAs. Chloramphenicol inhibits the peptidyl-transferase activity. This chemical binds to the loop of the 23S rRNA, which interacts with 3′-CCA end of all tRNAs. Moazed et al., Biochimie, 1987, 69, 879-884. Because linezolid resistance can be obtained by a altering the 23S rRNA genes, the oxazolidinone class of inhibitors appears to interact with catalytic rRNA. Kloss et al., J. Mol. Biol., 1999, 294, 93-101. The loop where these resistant inducing mutations are located is in proximity to the peptidyl-transferase catalytic site, possibly at the level of the pre-initiation complex.

Thiostrepton, a cyclic peptide based antibiotic, inhibits several reactions at the ribosomal GTPase center of the 50S ribosomal subunit. Evidence exists that thiostrepton acts by binding to the 23S rRNA component of the 50S subunit at the same site as the large ribosomal protein L11. The binding of L11 to the 23S rRNA causes a large conformation shift in the proteins tertiary structure. The binding of thiostrepton to the rRNA appears to cause an increase in the strength of the L11/23S rRNA interactions and prevents a conformational transition event in the L11 protein thereby stalling translation. Unfortunately, thiostrepton has very poor solubility, relatively high toxicity, and is not generally useful as an antibiotic. The mode of action of thiostrepton appears to be to stabilize a region of the 23S rRNA and by doing so prevent a structural transition in the L11 protein.

The oligonucleotide analog approach provides a useful alternative strategy in such applications by effectively subdividing large RNP's into small protein-free subdomains that, to some significant extent, recapitulate the functional properties of the analogous regions of the intact RNP. Implicit in this approach are the notions that the RNP (in this case the ribosome) is essentially an RNA machine, and that most, if not all, of the associated (ribosomal) proteins perform essentially a chaperonin function, by helping to guide the folding of the large and complexly structured rRNA. The feasibility of the oligonucleotide analog strategy has already been demonstrated with analogs of the decoding region of 16S rRNA, which recapitulate aminoglycoside antibiotic binding (and other) interactions of the small (30S) subunit of the ribosome. The present invention identifies subdomains of 23S rRNA that can act as targets for ribosome-targeted antimicrobial drug discovery.

Recent advances in genomics, molecular biology, and structural biology have highlighted how RNA molecules participate in or controls many of the events required to express proteins in cells. Rather than function as simple intermediaries, RNA molecules actively regulate their own transcription from DNA, splice and edit mRNA molecules and tRNA molecules, synthesize peptide bonds in the ribosome, catalyze the migration of nascent proteins to the cell membrane, and provide fine control over the rate of translation of messages. RNA molecules can adopt a variety of unique structural motifs that provide the framework required to perform these functions.

“Small” molecule therapeutics, which bind specifically to structured RNA molecules, are organic chemical molecules that are not polymers. “Small” molecule therapeutics include, for example, the most powerful naturally-occurring antibiotics. For example, the aminoglycoside and macrolide antibiotics are “small” molecules that bind to defined regions in ribosomal RNA (rRNA) structures and work, it is believed, by blocking conformational changes in the RNA required for protein synthesis. In addition, changes in the conformation of RNA molecules have been shown to regulate rates of transcription and translation of mRNA molecules. Small molecules are generally less than 10 kDa.

RNA molecules or groups of related RNA molecules are believed by Applicants to have regulatory regions that are used by the cell to control synthesis of proteins. The cell is believed to exercise control over both the timing and the amount of protein that is synthesized by direct, specific interactions with RNA. This notion is inconsistent with the impression obtained by reading the scientific literature on gene regulation, which is highly focused on transcription. The process of RNA maturation, transport, intracellular localization and translation are rich in RNA recognition sites that provide good opportunities for drug binding. Applicants' invention is directed, inter alia, to finding these regions of RNA molecules, in particular the 23S rRNA, in the microbial genome. Applicants' invention also makes use of combinatorial chemistry to make and/or screen, actually or virtually, a large number of chemical entities for their ability to bind and/or modulate these drug binding sites.

The determination of potential three dimensional structures of nucleic acids and their attendant structural motifs affords insights into areas such as the study of catalysis by RNA, RNA-RNA interactions, RNA-nucleic acid interactions, RNA-protein interactions, and the recognition of small molecules by nucleic acids. Four general approaches to the generation of model three dimensional structures of RNA have been demonstrated in the literature. All of these employ sophisticated molecular modelling and computational algorithms for the simulation of folding and tertiary interactions within target nucleic acids, such as RNA. Westhof and Altman (Proc. Natl. Acad. Sci., 1994, 91, 5133, incorporated herein by reference in its entirety) have described the generation of a three-dimensional working model of M1 RNA, the catalytic RNA subunit of RNase P from E. coli via an interactive computer modelling protocol. Leveraging the significant body of work in the area of cryo-electron microscopy (cryo-EM) and biochemical studies on ribosomal RNAs, Mueller and Brimacombe (J. Mol. Biol., 1997, 271, 524) have constructed a three dimensional model of E. coli 16S Ribosomal RNA. A method to model nucleic acid hairpin motifs has been developed based on a set of reduced coordinates for describing nucleic acid structures and a sampling algorithm that equilibrates structures using Monte Carlo (MC) simulations (Tung, Biophysical J., 1997, 72, 876, incorporated herein by reference in its entirety). MC-SYM is yet another approach to predicting the three dimensional structure of RNAs using a constraint-satisfaction method. Major et al., Proc. Natl. Acad. Sci., 1993, 90, 9408. The MC-SYM program is an algorithm based on constraint satisfaction that searches conformational space for all models that satisfy query input constraints, and is described in, for example, Cedergren et al., RNA Structure And Function, 1998, Cold Spring Harbor Lab. Press, p. 37-75. Three dimensional structures of RNA are produced by that method by the stepwise addition of nucleotide having one or several different conformations to a growing oligonucleotide model.

Westhof and Altman (Proc. Natl. Acad. Sci., 1994, 91, 5133) have described the generation of a three-dimensional working model of M1 RNA, the catalytic RNA subunit of RNase P from E. coli via an interactive computer modelling protocol. This modelling protocol incorporated data from chemical and enzymatic protection experiments, phylogenetic analysis, studies of the activities of mutants and the kinetics of reactions catalyzed by the binding of substrate to M1 RNA. Modelling was performed for the most part as described in the literature. Westhof et al., in “Theoretical Biochemistry and Molecular Biophysics,” Beveridge and Lavery (Eds.), Adenine, N.Y., 1990, 399. In general, starting with the primary sequence of M1 RNA, the stem-loop structures and other elements of secondary structure were created. Subsequent assembly of these elements into a three dimensional structure using a computer graphics station and FRODO (Jones, J. Appl. Crystallogr., 1978, 11, 268) followed by refinement using NUCLIN-NUCLSQ afforded a RNA model that had correct geometries, the absence of bad contacts, and appropriate stereochemistry. The model so generated was found to be consistent with a large body of empirical data on M1 RNA and opens the door for hypotheses about the mechanism of action of RNase P. The models generated by this method, however, are less well resolved that the structures determined via X-ray crystallography.

Mueller and Brimacombe (J. Mol. Biol., 1997, 271, 524, which is incorporated herein by reference in its entirety) have constructed a three dimensional model of E. coli 16S ribosomal RNA using a modelling program called ERNA-3D. This program generates three dimensional structures such as A-form RNA helices and single-strand regions via the dynamic docking of single strands to fit electron density obtained from low resolution diffraction data. After helical elements have been defined and positioned in the model, the configurations of the single strand regions is adjusted, so as to satisfy any known biochemical constraints such as RNA-protein cross-linking and foot-printing data.

A method to model nucleic acid hairpin motifs has been developed based on a set of reduced coordinates for describing nucleic acid structures and a sampling algorithm that equilibrates structures using Monte Carlo (MC) simulations. Tung, Biophysical J., 1997, 72, 876, incorporated herein by reference in its entirety. The stem region of a nucleic acid can be adequately modelled by using a canonical duplex formation. Using a set of reduced coordinates, an algorithm that is capable of generating structures of single stranded loops with a pair of fixed ends was created. This allows efficient structural sampling of the loop in conformational space. Combining this algorithm with a modified Metropolis Monte Carlo algorithm afforded a structure simulation package that simplifies the study of nucleic acid hairpin structures by computational means. Once the RNA subdomains have been identified, they can, if desired, be stabilized by the methods disclosed in U.S. Pat. No. 5,712,096.

While X-ray crystallography is a very powerful technique that can allow for the determination of some secondary and tertiary structure of biopolymeric targets (Erikson et al., Ann. Rep. in Med. Chem., 1992, 27, 271-289), this technique can be an expensive procedure and very difficult to accomplish. Crystallization of biopolymers is extremely challenging, difficult to perform at adequate resolution, and is often considered to be as much an art as a science. Further confounding the utility of X-ray crystal structures in the drug discovery process is the inability of crystallography to reveal insights into the solution-phase, and therefore the biologically relevant, structures of the targets of interest. Some analysis of the nature and strength of interaction between a ligand (agonist, antagonist, or inhibitor) and its target can be performed by ELISA (Kemeny and Challacombe, in ELISA and other Solid Phase Immunoassays: 1988), radioligand binding assays (Berson et al., Clin. 1968; Chard, in “An Introduction to Radioimmunoassay and Related Techniques,” 1982), surface-plasmon resonance (Karlsson et al., 1991, Jonsson et al., Biotechniques, 1991), or scintillation proximity assays (Udenfriend et al., Anal. Biochem., 1987), all cited previously. The radioligand binding assays are typically useful only when assessing the competitive binding of the unknown at the binding site for that of the radioligand and also require the use of radioactivity. The surface-plasmon resonance technique is more straightforward to use, but is also quite costly. Conventional biochemical assays of binding kinetics, and dissociation and association constants are also helpful in elucidating the nature of the target-ligand interactions.

Accordingly, one aspect of the invention identifies molecular interaction sites in 23S rRNA. These molecular interaction sites, which comprise secondary structural elements, are highly likely to give rise to significant therapeutic, regulatory, or other interactions with “small” molecules and the like. Another aspect of the invention is to compare molecular interaction sites of 23S rRNA with compounds proposed for interaction therewith.

Yet another aspect of the present invention is the establishment of databases of the numerical representations of three-dimensional structures of molecular interaction sites of 23S rRNA. Such databases libraries provide powerful tools for the elucidation of structure and interactions of molecular interaction sites with potential ligands and predictions thereof. Another aspect of the present invention is to provide a general method for the screening of combinatorial libraries comprising individual compounds or mixtures of compounds against 23S rRNA, so as to determine which components of the library bind to the target.

### SUMMARY OF THE INVENTION

The present invention is directed to identification of molecular interaction sites of 23S rRNA that comprise particular secondary structure.

The present invention is also directed to nucleic acid molecules, polynucleotides or oligonucleotides comprising the molecular interaction sites that can be used to screen, virtually or actually, combinatorial libraries of compounds that bind thereto.

The present invention is also directed to computer-readable medium comprising three dimensional representations of the structures of the molecular interaction sites.

The present invention is also directed to modulating the activity of 23S rRNA by contacting 23S rRNA or prokaryotic cells comprising the same with a compound identified by such virtual or actual screening.

The present invention is also directed to modulating prokaryotic cell growth comprising contacting a prokaryotic cell with a compound identified by such virtual or actual screening.

### BRIEF DESCRIPTION OF THE DRAWINGS

FIGS. 1A-1D depict representative secondary structures of a consensus 23S rRNA showing consensus sites 1-8 (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIGS. 2A-2E depict representative secondary structures of a consensus 23S rRNA showing consensus sites 9-18 and 22 (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIGS. 3A-3C depict representative secondary structures of a consensus 23S rRNA showing consensus sites 19-21 (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIGS. 4A-4C depict representative secondary structures of a consensus 23S rRNA showing consensus sites 23-27 (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIGS. 5A-5C depict representative secondary structures of a consensus 23S rRNA showing consensus sites 28-32 (nucleotides: capitalized letters=>95% conservation; small letters=90 to 95% conservation; ●−80 to 90% conservation; and ∘=<80% conservation; bonds: −=Watson-Crick bond; ● and ∘=non-cannonical bonds).

FIGS. 6A-6C depicts a representative secondary structure of a consensus 23S rRNA showing consensus sites 33-35 (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIG. 7 depicts a representative secondary structure of Candida albicans 23S rRNA (bonds: −=Watson-Crick bond; ● and ∘=non-cannonical bonds).

FIG. 8 depicts a representative secondary structure of Archaea consensus 23S rRNA (nucleotides: capitalized letters=>95% conservation; small letters =90 to 95% conservation; ●=80 to 90% conservation; and ∘=<80% conservation; bonds: −=Watson-Crick bond; ● and ∘=non-cannonical bonds).

FIG. 9 depicts a representative secondary structure of Haloarcula marismortui 23S rRNA (bonds: −=Watson-Crick bond; ● and ∘−non-cannonical bonds).

FIG. 10 depicts a representative secondary structure of chloroplast consensus 23S rRNA (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIG. 11 depicts a representative secondary structure of E. coli 23S rRNA (bonds: −=Watson-Crick bond; ● and ∘=non-cannonical bonds).

FIG. 12 depicts a representative secondary structure of fungal consensus 23S rRNA (nucleotides: capitalized letters=>95% conservation; small letters=90 to 95% conservation; ●=80 to 90% conservation; and ∘=<80% conservation; bonds: −=Watson-Crick bond; ● and ∘=non-cannonical bonds).

FIG. 13 depicts a representative secondary structure of Staphylococcus aureus 23S rRNA (bonds: −=Watson-Crick bond; ● and ∘=non-cannonical bonds).

FIGS. 14A and 14B depict representative secondary structures of region 116 of 23S rRNA in numerous species (bonds: −=Watson-Crick bond; ● and ∘=non-cannonical bonds).

FIG. 15 depicts representative secondary structures of region 120 of 23S rRNA in numerous species (bonds: −=Watson-Crick bond; ● and ∘=non-cannonical bonds).

FIG. 16 depicts representative secondary structures of region 165 of 23S rRNA in numerous species (bonds: −=Watson-Crick bond; ● and ∘=non-cannonical bonds).

FIG. 17 depicts representative secondary structures of region 113 of 23S rRNA in numerous species (bonds: −=Watson-Crick bond; ● and ∘=non-cannonical bonds).

FIG. 18 depicts representative secondary structures of region 114 of 23S rRNA in numerous species (bonds: −=Watson-Crick bond; ● and ∘=non-cannonical bonds).

FIG. 19 depicts representative secondary structures of region 117 of 23S rRNA in numerous species (bonds: −=Watson-Crick bond; ● and ∘=non-cannonical bonds).

FIG. 20 depicts representative secondary structures of region 115 of 23S rRNA in numerous species (bonds: −=Watson-Crick bond; ● and ∘−non-cannonical bonds).

FIG. 21 depicts representative secondary structures of region 110 of 23S rRNA in numerous species (bonds: −=Watson-Crick bond; ● and ∘=non-cannonical bonds).

### DESCRIPTION OF PREFERRED EMBODIMENTS OF THE INVENTION

The present invention is directed to, inter alia, identification of molecular interaction sites of 23S rRNA. Such molecular interaction sites comprise secondary structure capable of interacting with cellular components, such as factors and proteins required for translation and other cellular processes. Nucleic acid molecules or polynucleotides comprising the molecular interaction sites can be used to screen, virtually or actually, combinatorial libraries of compounds that bind thereto. The compounds identified by such screening are used to modulate the activity of 23S rRNA and, thus, can be used to modulate, either inhibit or stimulate, prokaryotic cell growth. Thus, novel drugs, agricultural chemicals, industrial chemicals and the like that operate through the modulation of 23S rRNA can be identified.

A number of procedures and protocols are preferably integrated to provide powerful drug and other biologically useful compound identification. Pharmaceuticals, veterinary drugs, agricultural chemicals, pesticides, herbicides, fungicides, industrial chemicals, research chemicals and many other beneficial compounds useful in pollution control, industrial biochemistry, and biocatalytic systems can be identified in accordance with embodiments of this invention. Novel combinations of procedures provide extraordinary power and versatility to the present methods. While it is preferred in some embodiments to integrate a number of processes developed by the assignee of the present application as will be set forth more fully herein, it should be recognized that other methodologies can be integrated herewith to good effect. Thus, while it is greatly advantageous to determine molecular binding sited on 23S rRNA in accordance with the teachings of this invention, the interactions of ligands and libraries of ligands with other 23S rRNA identified as being of interest may greatly benefit from other aspects of this invention. All such combinations are within the spirit of the invention.

One aspect of Applicants' invention is directed to identifying secondary structures in 23S rRNA termed “molecular interaction sites.” As used herein, “molecular interaction sites” are regions of 23S rRNA that have secondary structure. Molecular interaction sites can be conserved among a plurality of different taxonomic species of 23S rRNA. Molecular interaction sites are small, preferably less than 200 nucleotides, preferably less than 150 nucleotides, preferably less than 70 nucleotides, preferably less than 50 nucleotides, alternatively less than 30 nucleotides, independently folded, functional subdomains contained within a larger RNA molecule. Molecular interaction sites can contain both single-stranded and double-stranded regions. Thus, molecular interaction sites are capable of undergoing interaction with “small” molecules and otherwise, and are expected to serve as sites for interacting with “small” molecules, oligomers such as oligonucleotides, and other compounds in therapeutic and other applications. Molecular interaction sites also comprise a pocket for binding small molecules, drugs and the like.

The molecular interaction sites are present within at least 23S rRNA. In accordance with some embodiments of this invention, it will be appreciated that the 23S rRNAs having a molecular interaction site or sites may be derived from a number of sources. Thus, such 23S rRNAs can be identified by any means, rendered into three dimensional representations and employed for the identification of compounds that can interact with them to effect modulation of the 23S rRNA. In some embodiments, the molecular interaction sites that are identified in 23S rRNA are absent from eukaryotes, particularly humans, and, thus, can serve as sites for “small” molecule binding with concomitant modulation of the 23S rRNA of prokaryotic organisms without effecting human toxicity.

The molecular interaction sites can be identified by any means known to the skilled artisan. In some embodiments of the invention, the molecular interaction sites in 23S rRNA are identified according to the general methods described in International Publication WO 99/58719, which is incorporated herein by reference in its entirety. Briefly, a target 23S rRNA nucleotide sequence is chosen from among known sequences. Any 23S rRNA nucleotide sequence can be chosen. The nucleotide sequence of the target 23S rRNA is compared to the nucleotide sequences of a plurality of 23S rRNAs from different taxonomic species. At least one sequence region that is effectively conserved among the plurality of 23S rRNAs and the target 23S rRNA is identified. Such conserved region is examined to determine whether there is any secondary structure, and, for conserved regions having secondary structure, such secondary structure is identified.

In accordance with some embodiments of the invention, the nucleotide sequence of the target 23S rRNA is compared with the nucleotide sequences of a plurality of corresponding 23S rRNAs from different taxonomic species. Initial selection of a particular target nucleic acid can be based upon any functional criteria. 23S rRNA known to be involved in pathogenic genomes such as, for example, bacterial and yeast, are exemplary targets. Pathogenic bacteria and yeast are well known to those skilled in the art. Additional 23S rRNA targets can be determined independently or can be selected from publicly available prokaryotic genetic databases known to those skilled in the art. Databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet at, for example, ncbi.nlm.nih.gov/Omim/. CGAP, which is an interdisciplinary program to establish the information and technological tools required to decipher the molecular anatomy of a cancer cell, can be accessed through the world wide web of the Internet at, for example, ncbi.nlm.nih.gov/ncicgap/. Some of these databases may contain complete or partial nucleotide sequences. In addition, 23S rRNA targets can also be selected from private genetic databases. Alternatively, 23S rRNA targets can be selected from available publications or can be determined especially for use in connection with the present invention.

After a 23S rRNA target is selected or provided, the nucleotide sequence of the 23S rRNA target is determined and then compared to the nucleotide sequences of a plurality of 23S rRNAs from different taxonomic species. In one embodiment of the invention, the nucleotide sequence of the 23S rRNA target is determined by scanning at least one genetic database or is identified in available publications. Databases known and available to those skilled in the art include, for example, GenBank, and the like. These databases can be used in connection with searching programs such as, for example, Entrez, which is known and available to those skilled in the art, and the like. Entrez can be accessed through the world wide web of the Internet at, for example, ncbi.nlm.nih.gov/Entrez/. Preferably, the most complete nucleic acid sequence representation available from various databases is used. The GenBank database, which is known and available to those skilled in the art, can also be used to obtain the most complete nucleotide sequence. GenBank is the NIH genetic sequence database and is an annotated collection of all publicly available DNA sequences. GenBank is described in, for example, Nuc. Acids Res., 1998, 26, 1-7, which is incorporated herein by reference in its entirety, and can be accessed by those skilled in the art through the world wide web of the Internet at, for example, ncbi.nlm.nih.gov/Web/Genbank/ index.html. Alternatively, partial nucleotide sequences of 23S rRNA targets can be used when a complete nucleotide sequence is not available.

The nucleotide sequence of the 23S rRNA target is compared to the nucleotide sequences of a plurality of 23S rRNAs from different taxonomic species. A plurality of 23S rRNAs from different taxonomic species, and the nucleotide sequences thereof, can be found in genetic databases, from available publications, or can be determined especially for use in connection with the present invention. In one embodiment of the invention, the 23S rRNA target is compared to the nucleotide sequences of a plurality of 23S rRNAs from different taxonomic species by performing a sequence similarity search, an ortholog search, or both, such searches being known to persons of ordinary skill in the art.

The result of a sequence similarity search is a plurality of 23S rRNAs having at least a portion of their nucleotide sequences which are homologous to at least an 8 to 20 nucleotide region of the target 23S rRNA, referred to as the window region. Preferably, the plurality of 23S rRNAs comprise at least one portion which is at least 60% homologous to any window region of the target 23S rRNA. More preferably, the homology is at least 70%. More preferably, the homology is at least 80%. Most preferably, the homology is at least 90% or 95%. For example, the window size, the portion of the target 23S rRNA to which the plurality of sequences are compared, can be from about 8 to about 20, preferably from about 10 to about 15, most preferably from about 11 to about 12, contiguous nucleotides. The window size can be adjusted accordingly. A plurality of 23S rRNAs from different taxonomic species is then preferably compared to each likely window in the target 23S rRNA until all portions of the plurality of sequences is compared to the windows of the target 23S rRNA. Sequences of the plurality of 23S rRNAs from different taxonomic species which have portions which are at least 60%, preferably at least 70%, more preferably at least 80%, or most preferably at least 90% homologous to any window sequence of the target 23S rRNA are considered as likely homologous sequences.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet at, for example, ncbi.nlm.nih.gov/BLAST/. The GCG Package provides a local version of Blast that can be used either with public domain databases or with any locally available searchable database. GCG Package v.9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the world wide web of the Internet at, for example, gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Another toolkit capable of doing sequence similarity searching and data manipulation is SEALS, also from NCBI. This tool set is written in perl and C and can run on any computer platform that supports these languages. It is available for download, for example, at the world wide web of the Internet at ncbi.nlm.nih.gov/Walker/SEALS/. This toolkit provides access to Blast2 or gapped blast. It also includes a tool called tax_collector which, in conjunction with a tool called tax_break, parses the output of Blast2 and returns the identifier of the sequence most homologous to the query sequence for each species present. Another useful tool is feature2fasta which extracts sequence fragments from an input sequence based on the annotation.

Preferably, the plurality of 23S rRNAs from different taxonomic species which have homology to the target nucleic acid, as described above in the sequence similarity search, are further delineated so as to find orthologs of the target 23S rRNA therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing an ortholog search, an exhaustive list of homologous sequences from diverse organisms is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. The Blast Results-Relation database and the Annotations-Relational database are used in the Q-Compare protocol, which results in a list of ortholog sequences to compare in the interspecies sequence comparisons programs described below.

The above-described similarity searches provide results based on cut-off values, referred to as e-scores. E-scores represent the probability of a random sequence match within a given window of nucleotides. The lower the e-score, the better the match. One skilled in the art is familiar with e-scores. The user defines the e-value cut-off depending upon the stringency, or degree of homology desired, as described above. In some embodiments of the invention, it is preferred that any homologous nucleotide sequences of 23S rRNA that are identified not be present in the human genome.

In another embodiment of the invention, the sequences required are obtained by searching ortholog databases. One such database is Hovergen, which is a curated database of vertebrate orthologs. Ortholog sets may be exported from this database and used as is, or used as seeds for further sequence similarity searches as described above. Further searches may be desired, for example, to find invertebrate orthologs. Hovergen can be downloaded as a file transfer program at, for example, pbil.univ-lyonl.fr/pub/hovergen/. A database of prokaryotic orthologs, COGS, is available and can be used interactively through the world wide web of the Internet at, for example, ncbi.nlm.nih.gov/COG/.

After the orthologs or virtual transcripts described above are obtained through either the sequence similarity search or the ortholog search, at least one sequence region which is conserved among the plurality of 23S rRNAs from different taxonomic species and the target 23S rRNA is identified. Interspecies sequence comparisons can be performed using numerous computer programs which are available and known to those skilled in the art. Preferably, interspecies sequence comparison is performed using Compare, which is available and known to those skilled in the art. Compare is a GCG tool that allows pair-wise comparisons of sequences using a window/stringency criterion. Compare produces an output file containing points where matches of specified quality are found. These can be plotted with another GCG tool, DotPlot.

Alternatively, the identification of a conserved sequence region is performed by interspecies sequence comparisons using the ortholog sequences generated from Q-Compare in combination with CompareOverWins. Preferably, the list of sequences to compare, i.e., the ortholog sequences, generated from Q-Compare is entered into the CompareOverWins algorithm. Preferably, interspecies sequence comparisons are performed by a pair-wise sequence comparison in which a query sequence is slid over a window on the master target sequence. Preferably, the window is from about 9 to about 99 contiguous nucleotides.

Sequence homology between the window sequence of the target 23S rRNA and the query sequence of any of the plurality of 23S rRNAs obtained as described above, is preferably at least 60%, more preferably at least 70%, more preferably at least 80%, and most preferably at least 90% or 95%. The most preferable method of choosing the threshold is to have the computer automatically try all thresholds from 50% to 100% and choose a threshold based a metric provided by the user. One such metric is to pick the threshold such that exactly n hits are returned, where n is usually set to 3. This process is repeated until every base on the query nucleic acid, which is a member of the plurality of 23S rRNAs described above, has been compared to every base on the master target sequence. The resulting scoring matrix can be plotted as a scatter plot. Based on the match density at a given location, there may be no dots, isolated dots, or a set of dots so close together that they appear as a line. The presence of lines, however small, indicates primary sequence homology. Sequence conservation within 23S rRNA in divergent species is likely to be an indicator of conserved regulatory elements that are also likely to have a secondary structure. The results of the interspecies sequence comparison can be analyzed using MS Excel and visual basic tools in an entirely automated manner as known to those skilled in the art.

After at least one region that is conserved between the nucleotide sequence of the 23S rRNA target and the plurality of 23S rRNAs from different taxonomic species, preferably via the orthologs, is identified, the conserved region is analyzed to determine whether it contains secondary structure. Determining whether the identified conserved regions contain secondary structure can be performed by a number of procedures known to those skilled in the art. Determination of secondary structure is preferably performed by self complementarity comparison, alignment and covariance analysis, secondary structure prediction, or a combination thereof.

In one embodiment of the invention, secondary structure analysis is performed by alignment and covariance analysis. Numerous protocols for alignment and covariance analysis are known to those skilled in the art. Preferably, alignment is performed by ClustalW, which is available and known to those skilled in the art. ClustalW is a tool for multiple sequence alignment that, although not a part of GCG, can be added as an extension of the existing GCG tool set and used with local sequences. ClustalW can be accessed through the world wide web of the Internet at, for example, dot.imgen.bcm.tmc.edu:9331/multi-align/Options/clustalw.html. ClustalW is also described in Thompson, et al., Nuc. Acids Res., 1994, 22, 4673-4680, which is incorporated herein by reference in its entirety. These processes can be scripted to automatically use conserved UTR regions identified in earlier steps. Seqed, a UNIX command line interface available and known to those skilled in the art, allows extraction of selected local regions from a larger sequence. Multiple sequences from many different species can be clustered and aligned for further analysis.

In another embodiment of the invention, the output of all possible pair-wise CompareOverWindows comparisons are compiled and aligned to a reference sequence using a program called AlignHits, a program that can be reproduced by one skilled in the art. One purpose of this program is to map all hits made in pair-wise comparisons back to the position on a reference sequence. This method combining CompareOverWindows and AlignHits provides more local alignments (over 20-100 bases) than any other algorithm. This local alignment is required for the structure finding routines described later such as covariation or RevComp. This algorithm writes a fasta file of aligned sequences. It is important to differentiate this from using ClustalW by itself, without CompareOverWindows and AlignHits.

Covariation is a process of using phylogenetic analysis of primary sequence information for consensus secondary structure prediction. Covariation is described in the following references, each of which is incorporated herein by reference in their entirety: Gutell et al., “Comparative Sequence Analysis Of Experiments Performed During Evolution” In Ribosomal RNA Group I Introns, Green, Ed., Austin: Landes, 1996; Gautheret et al., Nuc. Acids Res., 1997, 25, 1559-1564; Gautheret et al., RNA, 1995, 1, 807-814; Lodmell et al., Proc. Natl. Acad. Sci. USA, 1995, 92, 10555-10559; Gautheret et al., J. Mol. Biol., 1995, 248, 27-43; Gutell, Nuc. Acids Res., 1994, 22, 3502-3517; Gutell, Nuc. Acids Res., 1993, 21, 3055-3074; Gutell, Nuc. Acids Res., 1993, 21, 3051-3054; Woese, Proc. Natl. Acad. Sci. USA, 1989, 86, 3119-3122; and Woese et al., Nuc. Acids Res., 1980, 8, 2275-2293, each of which is incorporated herein by reference in its entirety. Preferably, covariance software is used for covariance analysis. Preferably, Covariation, a set of programs for the comparative analysis of RNA structure from sequence alignments, is used. Covariation uses phylogenetic analysis of primary sequence information for consensus secondary structure prediction. Covariation can be obtained through the world wide web of the Internet at, for example, mbio.ncsu.edu/RNaseP/info/programs/programs.html. A complete description of a version of the program has been published (Brown, J. W. 1991, Phylogenetic analysis of RNA structure on the Macintosh computer. CABIOS 7:391-393). The current version is v4.1, which can perform various types of covariation analysis from RNA sequence alignments, including standard covariation analysis, the identification of compensatory base-changes, and mutual information analysis. The program is well-documented and comes with extensive example files. It is compiled as a stand-alone program; it does not require Hypercard (although a much smaller ‘stack’ version is included). This program will run in any Macintosh environment running MacOS v7.1 or higher. Faster processor machines (68040 or PowerPC) is suggested for mutual information analysis or the analysis of large sequence alignments.

In another embodiment of the invention, secondary structure analysis is performed by secondary structure prediction. There are a number of algorithms that predict RNA secondary structures based on thermodynamic parameters and energy calculations. Preferably, secondary structure prediction is performed using either M-fold or RNA Structure 2.52. M-fold can be accessed through the world wide web of the Internet at, for example, ibc.wustl.edu/-zuker/ma/form2.cgi or can be downloaded for local use on UNIX platforms. M-fold is also available as a part of GCG package. RNA Structure 2.52 is a windows adaptation of the M-fold algorithm and can be accessed through the world wide web of the Internet at, for example, 128.151.176.70/RNAstructure.html.

In another embodiment of the invention, secondary structure analysis is performed by self complementarity comparison. Preferably, self complementarity comparison is performed using Compare, described above. More preferably, Compare can be modified to expand the pairing matrix to account for G-U or U-G basepairs in addition to the conventional Watson-Crick G-C/C-G or A-U/U-A pairs. Such a modified Compare program (modified Compare) begins by predicting all possible base-pairings within a given sequence. As described above, a small but conserved region is identified based on primary sequence comparison of a series of orthologs. In modified Compare, each of these sequences is compared to its own reverse complement. Allowable base-pairings include Watson-Crick A-U, G-C pairing and non-canonical G-U pairing. An overlay of such self complementarity plots of all available orthologs, and selection for the most repetitive pattern in each, results in a minimal number of possible folded configurations. These overlays can then used in conjunction with additional constraints, including those imposed by energy considerations described above, to deduce the most likely secondary structure.

In another embodiment of the invention, the output of AlignHits is read by a program called RevComp. This program could be reproduced by one skilled in the art. One purpose of this program is to use base pairing rules and ortholog evolution to predict RNA secondary structure. RNA secondary structures are composed of single stranded regions and base paired regions, called stems. Since structure conserved by evolution is searched, the most probable stem for a given alignment of ortholog sequences is the one which could be formed by the most sequences. Possible stem formation or base pairing rules is determined by, for example, analyzing base pairing statistics of stems which have been determined by other techniques such as NMR. The output of RevComp is a sorted list of possible structures, ranked by the percentage of ortholog set member sequences which could form this structure. Because this approach uses a percentage threshold approach, it is insensitive to noise sequences. Noise sequences are those that either not true orthologs, or sequences that made it into the output of AlignHits due to high sequence homology even though they do not represent an example of the structure which is searched. A very similar algorithm is implemented using Visual basic for Applications (VBA) and Microsoft Excel to be run on PCs, to generate the reverse complement matrix view for the given set of sequences.

A result of the secondary structure analysis described above, whether performed by alignment and covariance, self complementarity analysis, secondary structure predictions, such as using M-fold or otherwise, is the identification of secondary structure in the conserved regions among the target 23S rRNA and the plurality of 23S rRNAs from different taxonomic species. Exemplary secondary structures that may be identified include, but are not limited to, bulges, loops, stems, hairpins, knots, triple interacts, cloverleafs, or helices, or a combination thereof. Alternatively, new secondary structures may be identified.

The present invention is also directed to nucleic acid molecules, such as polynucleotides and oligonucleotides, comprising a molecular interaction site present in 23S rRNA. Nucleic acid molecules include the physical compounds themselves as well as in silico representations of the same. Thus, the nucleic acid molecules are derived from 23S rRNA. The molecular interaction site serves as a binding site for at least one molecule which, when bound to the molecular interaction site, modulates the expression of the 23S rRNA in a cell. The nucleotide sequence of the polynucleotide is selected to provide the secondary structure of the molecular interaction sites described in grater detail in the Examples. The nucleotide sequence of the polynucleotide is preferably the nucleotide sequence of the target 23S rRNAs, described above. Alternatively, the nucleotide sequence is preferably the nucleotide sequence of 23S rRNAs from a plurality of different taxonomic species which also contain the molecular interaction site.

The polynucleotides of the invention comprise the molecular interaction sites of the 23S rRNA. Thus, the polynucleotides of the invention comprise the nucleotide sequences of the molecular interaction sites. In addition, the polynucleotides can comprise up to 50, more preferably up to 40, more preferably up to 30, more preferably up to 20, and most preferably up to 10 additional nucleotides at either the 5′ or 3′, or combination thereof, ends of each polynucleotide. Thus, for example, if a molecular interaction site comprises 25 nucleotides, the polynucleotide can comprise up to 75 nucleotides. The nucleotides that are in addition to those present in the molecular interaction site are selected to preserve the secondary structure of the molecular interaction site. One skilled in the art can select such additional nucleotides so as to conserve the secondary structure. The polynucleotides can comprise either RNA or DNA or can be chimeric RNA/DNA. The polynucleotides can comprise modified bases, sugars and backbones that are well known to the skilled artisan. Further, a single polynucleotide can comprise a plurality of molecular interaction sites. In addition, a plurality of polynucleotides can, together, comprise a single molecular interaction site. Alternatively, when a plurality of polynucleotides together comprise a molecular interaction site, one skilled in the art can attach the polynucleotides to one another, thus, forming a single polynucleotide.

The portion of the polynucleotide comprising the molecular interaction site can comprise one or more deletions, insertions and substitutions. Stems, terminal loops, bulges, internal loops, and dangling regions can comprise one or more deletions, insertions and substitutions. Thus, for example, a terminal loop of a molecular interaction site that consists of 10 nucleotides can be modified to contain one or more insertions, deletions or substitutions, thus, resulting in a shortening or lengthening of the stem preceding the terminal loop. In addition, unpaired, dangling nucleotides that are adjacent to, for example, a double-stranded region can be deleted or can be basepaired with the addition of another nucleotide, thus, lengthening the stem. In addition, nucleotide base pairings within a stem can also be substituted, deleted, or inserted. Thus, for example, an A-U basepair within a stem portion of a molecular interaction site can be replaced with a G-C basepair. Further, non-canonical base pairing (e.g., G-A, C-T, G-U, etc.) can also be present within the polynucleotide. Thus, polynucleotides having at least 70%, more preferably 80%, more preferably 90%, more preferably 95%, and most preferably 99% homology with the molecular interaction sites, such as those set forth in the Examples below, are included within the scope of the invention. Percent homology can be determined by, for example, the Gap program (Wisconsin Sequence Analysis Package, Version 8 for Unix, Genetics Computer Group, University Research Park, Madison Wis.), using the default settings, which uses the algorithm of Smith and Waterman (Adv. Appl. Math., 1981, 2, 482-489, which is incorporated herein by reference in its entirety).

The present invention is also directed to the purified and isolated nucleic acid molecules, or polynucleotides, described above, that are present within 23S rRNA. The polynucleotides comprising the molecular interaction site mimic the portion of the 23S rRNA comprising the molecular interaction site.

Polynucleotides, and modifications thereof, are well known to those skilled in the art. The polynucleotides of the invention can be used, for example, as research reagents to detect, for example, naturally occurring molecules that bind the molecular interaction sites. Alternatively, the polynucleotides of the invention can be used to screen, either actually or virtually, small molecules that bind the molecular interaction sites, as described below in greater detail. Virtual generation of compounds and screening thereof for binding to molecular interaction sites is described in, for example, International Publication WO 99/58947, which is incorporated herein by reference in its entirety. The polynucleotides of the invention can also be used as decoys to compete with naturally-occurring molecular interaction sites within a cell for research, diagnostic and therapeutic applications. In particular, the polynucleotides can be used in, for example, therapeutic applications to inhibit bacterial growth. Molecules that bind to the molecular interaction site modulate, either by augmenting or diminishing, the function of 23S rRNA in translation. The polynucleotides can also be used in agricultural, industrial and other applications.

The present invention is also directed to compositions comprising at least one polynucleotide described above. In some embodiments of the invention, two polynucleotides are included within a composition. The compositions of the invention can optionally comprise a carrier. A “carrier” is an acceptable solvent, diluent, suspending agent or any other inert vehicle for delivering one or more nucleic acids to an animal, and are well known to those skilled in the art. The carrier can be a pharmaceutically acceptable carrier. The carrier can be liquid or solid and is selected, with the planned manner of administration in mind, so as to provide for the desired bulk, consistency, etc., when combined with the other components of the composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

The present invention is also directed to methods of identifying compounds that bind to a molecular interaction site of 23S rRNA comprising providing a numerical representation of the three-dimensional structure of the molecular interaction site and providing a compound data set comprising numerical representations of the three dimensional structures of a plurality of organic compounds. The numerical representation of the molecular interaction site is then compared with members of the compound data set to generate a hierarchy of organic compounds ranked in accordance with the ability of the organic compounds to form physical interactions with the molecular interaction site.

The present invention is also directed to methods of identifying compounds that bind to a molecular interaction site of 23S rRNA, or a polynucleotide comprising the same. In some embodiments of the invention, compounds that bind to a molecular interaction site of 23S rRNA, or a polynucleotide comprising the same, are identified according to the general methods described in International Publication WO 99/58947, which is incorporated herein by reference in its entirety. Briefly, the methods comprise providing a numerical representation of the three dimensional structure of the molecular interaction site, or a polynucleotide comprising the same, providing a compound data set comprising numerical representations of the three dimensional structures of a plurality of organic compounds, comparing the numerical representation of the molecular interaction site with members of the compound data set to generate a hierarchy of organic compounds which is ranked in accordance with the ability of the organic compounds to form physical interactions with the molecular interaction site.

While there are a number of ways to characterize binding between molecular interaction sites and ligands, such as for example, organic compounds, methodologies are described in International Publications WO 99/58719, WO 99/59061, WO 99/58722, WO 99/45150, WO 99/58474, and WO 99/58947, each of which is assigned to the assignee of the present inventions, and each of which is incorporated by reference herein in their entirety.

In addition, the present invention is also directed to three dimensional representations of the nucleic acid molecules, and compositions comprising the same, described above. The three dimensional structure of a molecular interaction site of 23S rRNA can be manipulated as a numerical representation. The three dimensional representations, i.e., in silico (e.g. in computer-readable form) representations can be generated by methods disclosed in, for example, International Publication WO 99/58947, which is incorporated herein by reference in its entirety. Briefly, the three dimensional structure of a molecular interaction site, preferably of an RNA, can be manipulated as a numerical representation. Computer software that provides one skilled in the art with the ability to design molecules based on the chemistry being performed and on available reaction building blocks is commercially available. Software packages such as, for example, Sybyl/Base (Tripos, St. Louis, Mo.), Insight II (Molecular Simulations, San Diego, Calif.), and Sculpt (MDL Information Systems, San Leandro, Calif.) provide means for computational generation of structures. These software products also provide means for evaluating and comparing computationally generated molecules and their structures. In silico collections of molecular interaction sites can be generated using the software from any of the above-mentioned vendors and others which are or may become available. The three dimensional representations can be used, for example, to dock the molecule(s) to potential therapeutic compounds. Thus, the three dimensional representations can be used in drug screening procedures. Accordingly, the nucleic acid molecules and compositions comprising the same of the present invention include the three dimensional representations of the same.

A set of structural constraints for the molecular interaction site of the 23S rRNA can be generated from biochemical analyses such as, for example, enzymatic mapping and chemical probes, and from genomics information such as, for example, covariance and sequence conservation. Information such as this can be used to pair bases in the stem or other region of a particular secondary structure. Additional structural hypotheses can be generated for noncanonical base pairing schemes in loop and bulge regions. A Monte Carlo search procedure can sample the possible conformations of the 23S rRNA consistent with the program constraints and produce three dimensional structures.

Reports of the generation of three dimensional, in silico representations are available from the standpoint of library design, generation, and screening against protein targets. Likewise, some efforts in the area of generating RNA models have been reported in the literature. However, there are no reports on the use of structure-based design approaches to query in silico representations of organic molecules, “small” molecules, polynucleotides or other nucleic acids, with three dimensional, in silico, representations of 23S rRNA structures. The present invention preferably employs computer software that allows the construction of three dimensional models of 23S rRNA structure, the construction of three dimensional, in silico representations of a plurality of organic compounds, “small” molecules, polymeric compounds, polynucleotides and other nucleic acids, screening of such in silico representations against 23S rRNA molecular interaction sites in silico, scoring and identifying the best potential binders from the plurality of compounds, and finally, synthesizing such compounds in a combinatorial fashion and testing them experimentally to identify new ligands for such 23S rRNA targets.

The molecules that may be screened by using the methods of this invention include, but are not limited to, organic or inorganic, small to large molecular weight individual compounds, and combinatorial mixture or libraries of ligands, inhibitors, agonists, antagonists, substrates, and biopolymers, such as peptides or polynucleotides. Combinatorial mixtures include, but are not limited to, collections of compounds, and libraries of compounds. These mixtures may be generated via combinatorial synthesis of mixtures or via admixture of individual compounds. Collections of compounds include, but are not limited to, sets of individual compounds or sets of mixtures or pools of compounds. These combinatorial libraries may be obtained from synthetic or from natural sources such as, for example to, microbial, plant, marine, viral and animal materials. Combinatorial libraries include at least about twenty compounds and as many as a thousands of individual compounds and potentially even more. When combinatorial libraries are mixtures of compounds these mixtures typically contain from 20 to 5000 compounds preferably from 50 to 1000, more preferably from 50 to 100. Combinations of from 100 to 500 are useful as are mixtures having from 500 to 1000 individual species. Typically, members of combinatorial libraries have molecular weight less than about 10,000 Da, more preferably less than 7,500 Da, and most preferably less than 5000 Da.

A significant advance in the area of virtual screening was the development of a software program called DOCK that allows structure-based database searches to find and identify the interactions of known molecules to a receptor of interest (Kuntz et al., Acc. Chem. Res., 1994, 27, 117; Geschwend and Kuntz, J. Compt.-Aided Mol. Des., 1996, 10, 123). DOCK allows the screening of molecules, whose 3D structures have been generated in silico, but for which no prior knowledge of interactions with the receptor is available. DOCK, therefore, provides a tool to assist in discovering new ligands to a receptor of interest. DOCK can thus be used for docking the compounds prepared according to the methods of the present invention to desired target molecules. Implementation of DOCK is described in, for example, International Publication WO 99/58947, which is incorporated herein by reference in its entirety.

In some embodiments of the invention, an automated computational search algorithm, such as those described above, is used to predict all of the allowed three dimensional molecular interaction site structures from 23S rRNA, which are consistent with the biochemical and genomic constraints specified by the user. Based, for example, on their root-mean-squared deviation values, these structures are clustered into different families. A representative member or members of each family can be subjected to further structural refinement via molecular dynamics with explicit solvent and cations.

Structural enumeration and representation by these software programs is typically done by drawing molecular scaffolds and substituents in two dimensions. Once drawn and stored in the computer, these molecules may be rendered into three dimensional structures using algorithms present within the commercially available software. Preferably, MC-SYM is used to create three dimensional representations of the molecular interaction site. The rendering of two dimensional structures of molecular interaction sites into three dimensional models typically generates a low energy conformation or a collection of low energy conformers of each molecule. The end result of these commercially available programs is the conversion of a 23S rRNA sequence containing a molecular interaction site into families of similar numerical representations of the three dimensional structures of the molecular interaction site. These numerical representations form an ensemble data set.

The three dimensional structures of a plurality of compounds, preferably “small” organic compounds, can be designated as a compound data set comprising numerical representations of the three dimensional structures of the compounds. “Small” molecules in this context refers to non-oligomeric organic compounds. Two dimensional structures of compounds can be converted to three dimensional structures, as described above for the molecular interaction sites, and used for querying against three dimensional structures of the molecular interaction sites. The two dimensional structures of compounds can be generated rapidly using structure rendering algorithms commercially available. The three dimensional representation of the compounds which are polymeric in nature, such as polynucleotides or other nucleic acids structures, may be generated using the literature methods described above. A three dimensional structure of “small” molecules or other compounds can be generated and a low energy conformation can be obtained from a short molecular dynamics minimization. These three dimensional structures can be stored in a relational database. The compounds upon which three dimensional structures are constructed can be proprietary, commercially available, or virtual.

In some embodiments of the invention, a compound data set comprising numerical representations of the three dimensional structure of a plurality of organic compounds is provided by, for example, Converter (MSI, San Diego) from two dimensional compound libraries generated by, for example, a computer program modified from a commercial program. Other suitable databases can be constructed by converting two dimensional structures of chemical compounds into three dimensional structures, as described above. The end result is the conversion of a two dimensional structure of organic compounds into numerical representations of the three dimensional structures of a plurality of organic compounds. These numerical representations are presented as a compound data set.

After both the numerical representations of the three-dimensional structure of the polynucleotides comprising the molecular interaction sites and the compound data set comprising numerical representations of the three dimensional structures of a plurality of organic compounds are obtained, the numerical representations of the molecular interaction sites are compared with members of the compound data set to generate a hierarchy of the organic compounds. The hierarchy is ranked in accordance with the ability of the organic compounds to form physical interactions with the molecular interaction site. Preferably, the comparing is carried out seriatim upon the members of the compound data set. In accordance with some embodiments, the comparison can be performed with a plurality of polynucleotides comprising molecular interaction sites at the same time.

A variety of theoretical and computational methods are known by those skilled in the art to study and optimize the interactions of “small” molecules or organic compounds with biological targets such as nucleic acids. These structure-based drug design tools have been very useful in modelling the interactions of proteins with small molecule ligands and in optimizing these interactions. Typically this type of study has been performed when the structure of the protein receptor was known by querying individual small molecules, one at a time, against this receptor. Usually these small molecules had either been co-crystallized with the receptor, were related to other molecules that had been co-crystallized or were molecules for which some body of knowledge existed concerning their interactions with the receptor. DOCK, as described above, can be used to find and identify molecules that are expected to bind to polynucleotides comprising the molecular interaction sites and, hence, 23S rRNA of interest. DOCK 4.0 is commercially available from the Regents of the University of California. Equivalent programs are also comprehended in the present invention.

The DOCK program has been widely applied to protein targets and the identification of ligands that bind to them. Typically, new classes of molecules that bind to known targets have been identified, and later verified by in vitro experiments. The DOCK software program consists of several modules, including SPHGEN (Kuntz et al., J. Mol. Biol., 1982, 161, 269) and CHEMGRID (Meng et al., J. Comput. Chem., 1992, 13, 505, each of which is incorporated herein by reference in its entirety). SPHGEN generates clusters of overlapping spheres that describe the solvent-accessible surface of the binding pocket within the target receptor. Each cluster represents a possible binding site for small molecules. CHEMGRID precalculates and stores in a grid file the information necessary for force field scoring of the interactions between binding molecule and target 23S rRNA. The scoring function approximates molecular mechanics interaction energies and consists of van der Waals and electrostatic components. DOCK uses the selected cluster of spheres to orient ligands molecules in the targeted site on 23S rRNA. Each molecule within a previously generated three dimensional database is tested in thousands of orientations within the site, and each orientation is evaluated by the scoring function. Only that orientation with the best score for each compound so screened is stored in the output file. Finally, all compounds of the database are ranked in a hierarchy in order of their scores and a collection of the best candidates may then be screened experimentally.

Using DOCK, numerous ligands have been identified for a variety of protein targets. Recent efforts in this area have resulted in reports of the use of DOCK to identify and design small molecule ligands that exhibit binding specificity for nucleic acids such as RNA double helices. While RNA plays a significant role in many diseases such as AIDS, viral and bacterial infections, few studies have been made on small molecules capable of specific RNA binding. Compounds possessing specificity for the RNA double helix, based on the unique geometry of its deep major groove, were identified using the DOCK methodology. Chen et al., Biochemistry, 1997, 36, 11402 and Kuntz et al., Acc. Chem. Res., 1994, 27, 117. Recently, the application of DOCK to the problem of ligand recognition in DNA quadruplexes has been reported. Chen et al., Proc. Natl. Acad. Sci., 1996, 93, 2635.

Preferably, individual compounds are designated as mol files, for example, and combined into a collection of in silico representations using an appropriate chemical structure program or equivalent software. These two dimensional mol files are exported and converted into three dimensional structures using commercial software such as Converter (Molecular Simulations Inc., San Diego) or equivalent software, as described above. Atom types suitable for use with a docking program such as DOCK or QXP are assigned to all atoms in the three dimensional mol file using software such as, for example, Babel, or with other equivalent software.

A low-energy conformation of each molecule is generated with software such as Discover (MSI, San Diego). An orientation search is performed by bringing each compound of the plurality of compounds into proximity with the molecular interaction site in many orientations using DOCK or QXP. A contact score is determined for each orientation, and the optimum orientation of the compound is subsequently used. Alternatively, the conformation of the compound can be determined from a template conformation of the scaffold determined previously.

The interaction of a plurality of compounds and molecular interaction sites is examined by comparing the numerical representations of the molecular interaction sites with members of the compound data set. Preferably, a plurality of compounds such as those generated by a computer program or otherwise, is compared to the molecular interaction site and undergoes random “motions” among the dihedral bonds of the compounds. Preferably about 20,000 to 100,000 compounds are compared to at least one molecular interaction site. Typically, 20,000 compounds are compared to about five molecular interaction sites and scored. Individual conformations of the three dimensional structures are placed at the target site in many orientations. Moreover, during execution of the DOCK program, the compounds and molecular interaction sites are allowed to be “flexible” such that the optimum hydrogen bonding, electrostatic, and van der Waals contacts can be realized. The energy of the interaction is calculated and stored for 10-15 possible orientations of the compounds and molecular interaction sites. QXP methodology allows true flexibility in both the ligand and target and is presently preferred.

The relative weights of each energy contribution are updated constantly to insure that the calculated binding scores for all compounds reflect the experimental binding data. The binding energy for each orientation is scored on the basis of hydrogen bonding, van der Waals contacts, electrostatics, solvation/desolvation, and the quality of the fit. The lowest-energy van der Waals, dipolar, and hydrogen bonding interactions between the compound and the molecular interaction site are determined, and summed. In some embodiments, these parameters can be adjusted according to the results obtained empirically. The binding energies for each molecule against the target are output to a relational database. The relational database contains a hierarchy of the compounds ranked in accordance with the ability of the compounds to form physical interactions with the molecular interaction site. The higher ranked compounds are better able to form physical interactions with the molecular interaction site.

In another embodiment, the highest ranking, i.e., the best fitting compounds, are selected for synthesis. In some embodiments of the invention, those compounds which are likely to have desired binding characteristics based on binding data are selected for synthesis. Preferably the highest ranking 5% are selected for synthesis. More preferably, the highest ranking 10% are selected for syntheses. Even more preferably, the highest ranking 20% are selected for synthesis. The synthesis of the selected compounds can be automated using a parallel array synthesizer or prepared using solution-phase or other solid-phase methods and instruments. In addition, the interaction of the highly ranked compounds with the nucleic acid containing the molecular interaction site is assessed as described below.

The interaction of the highly ranked organic compounds with the polynucleotide comprising the 23S rRNA molecular interaction site can be assessed by numerous methods known to those skilled in the art. For example, the highest ranking compounds can be tested for activity in high-throughput (HTS) functional and cellular screens. HTS assays can be determined by scintillation proximity, precipitation, luminescence-based formats, filtration based assays, colorometric assays, and the like. Lead compounds can then be scaled up and tested in animal models for activity and toxicity. The assessment preferably comprises mass spectrometry of a mixture of the 23S rRNA polynucleotide and at least one of the compounds or a functional bioassay.

Certain evaluation techniques employing mass spectroscopy are disclosed in International Publication WO 99/45150, which is incorporated herein by reference in its entirety, as exemplary of certain useful and mass spectrometric techniques for use herewith. It is to be specifically understood, however, that it is not essential that these particular mass spectrometric techniques be employed in order to perform the present invention. Rather, any evaluative technique may be undertaken so long as the objectives of the present invention are maintained.

In some embodiments of the invention, the highest ranking 20% of compounds from the hierarchy generated using the DOCK program or QXP are used to generate a further data set of three dimensional representations of organic compounds comprising compounds which are chemically related to the compounds ranking high in the hierarchy. Although the best fitting compounds are likely to be in the highest ranking 1%, additional compounds, up to about 20%, are selected for a second comparison so as to provide diversity (ring size, chain length, functional groups). This process insures that small errors in the molecular interaction sites are not propagated into the compound identification process. The resulting structure/score data from the highest ranking 20%, for example, is studied mathematically (clustered) to find trends or features within the compounds which enhance binding. The compounds are clustered into different groups. Chemical synthesis and screening of the compounds, described above, allows the computed DOCK or QXP scores to be correlated with the actual binding data. After the compounds have been prepared and screened, the predicted binding energy and the observed Kd values are correlated for each compound.

The results are used to develop a predictive scoring scheme, which weighs various factors (steric, electrostatic) appropriately. The above strategy allows rapid evaluation of a number of scaffolds with varying sizes and shapes of different functional groups for the high ranked compounds. In this manner, a further data set of representations of organic compounds comprising compounds which are chemically related to the organic compounds which rank high in the hierarchy can be compared to the numerical representations of the molecular interaction site to determine a further hierarchy ranked in accordance with the ability of the organic compounds to form physical interactions with the molecular interaction site. In this manner, the further data set of representations of the three dimensional structures of compound which are related to the compounds ranked high in the hierarchy are produced and have, in effect, been optimized by correlating actual binding with virtual binding. The entire cycle can be iterated as desired until the desired number of compounds highest in the hierarchy are produced.

Compounds which have been determined to have affinity and specificity for a target biomolecule, especially a target 23S rRNA or which otherwise have been shown to be able to bind to the target 23S rRNA to effect modulation thereof, can, in accordance with some embodiments of this invention, be tagged or labelled in a detectable fashion. Such labelling may include all of the labelling forms known to persons of skill in the art such as fluorophore, radiolabel, enzymatic label and many other forms. Such labelling or tagging facilitates detection of molecular interaction sites and permits facile mapping of chromosomes and other useful processes.

In order that the invention disclosed herein may be more efficiently understood, examples are provided below. It should be understood that these examples are for illustrative purposes only and are not to be construed as limiting the invention in any manner. Various modifications of the invention, in addition to those described herein, will be apparent to those skilled in the art from the foregoing description. Such modifications are also intended to fall within the scope of the appended claims. In addition, the disclosures of each patent, patent application, and publication cited or described in this document are incorporated herein by reference in their entirety.

### EXAMPLES

### Example 1

### Selection of 23S rRNA

To illustrate the strategy for identifying molecular interaction sites for small molecules, the 23S rRNA was used. The structure of the entire 23S rRNA molecule is described in, for example, Ban et al., Science, 2000, 289, 905-920, which is incorporated herein by reference in its entirety.

### Example 2

### Molecular Interaction Sites In 23S rRNA

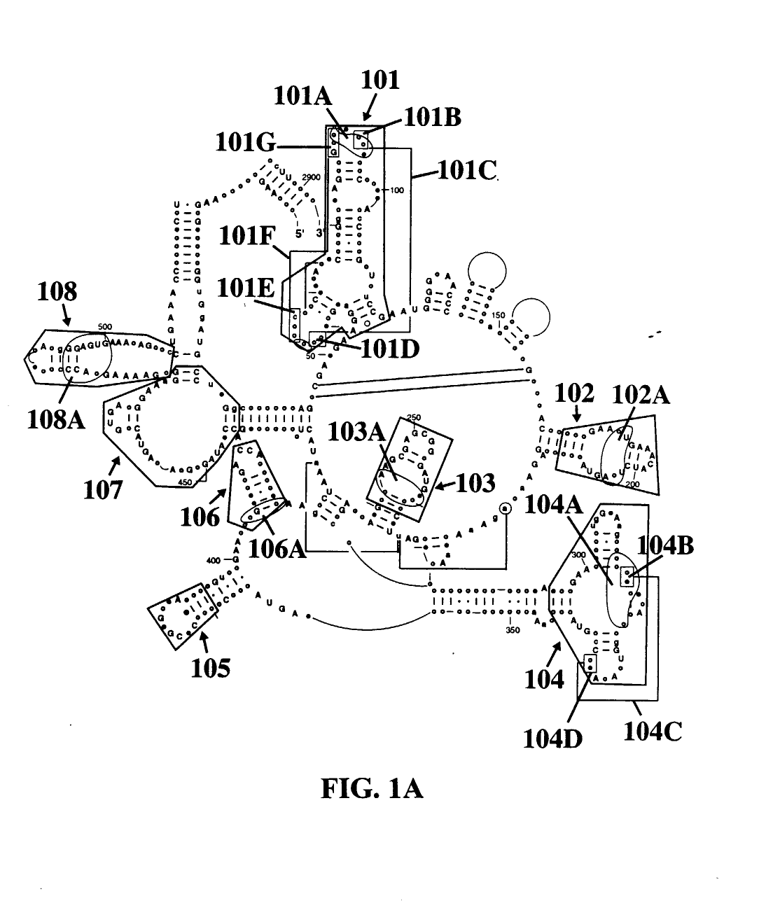

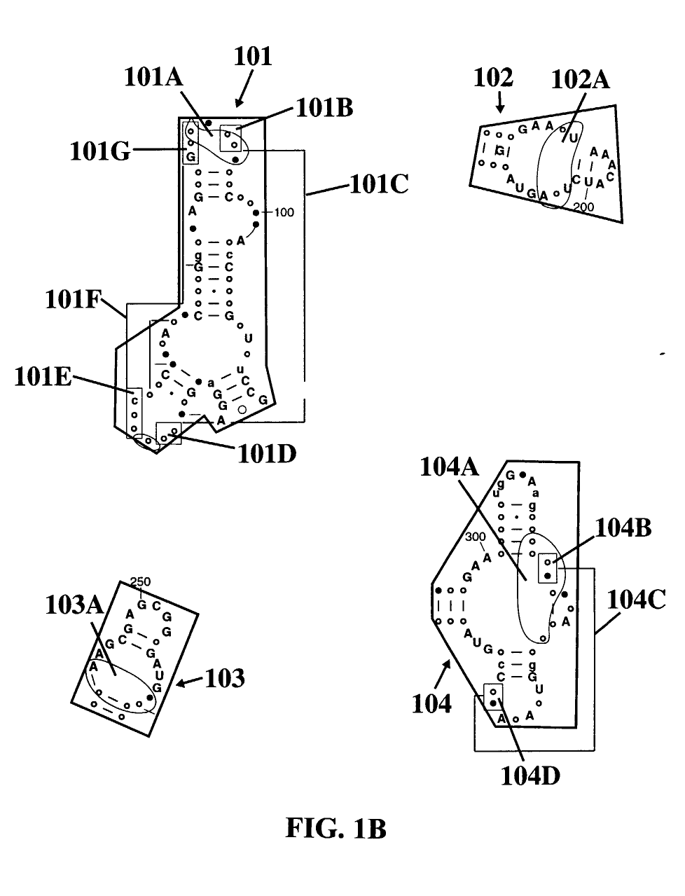

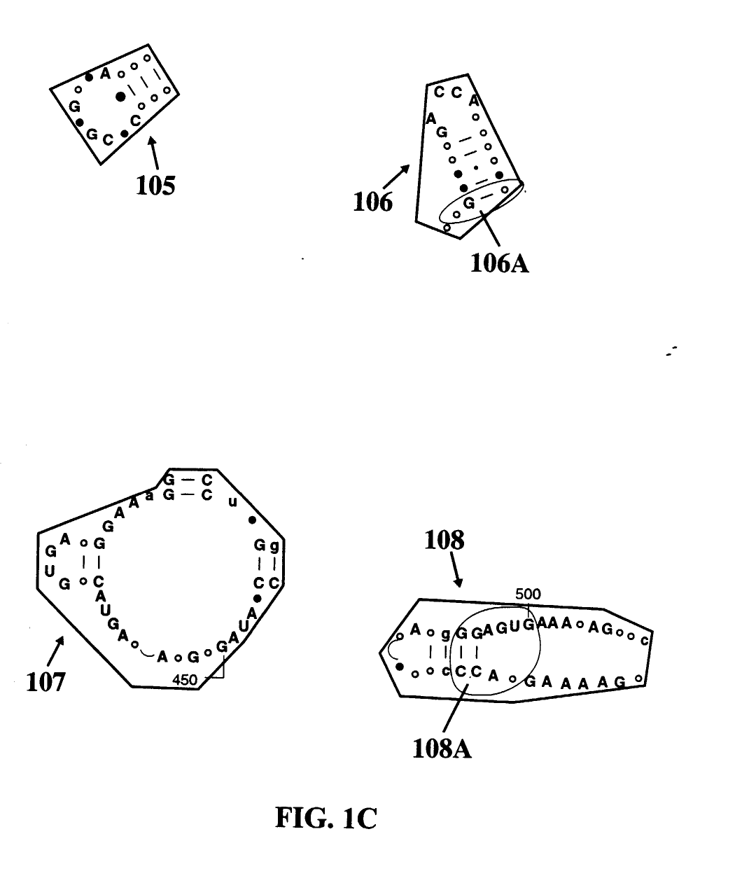

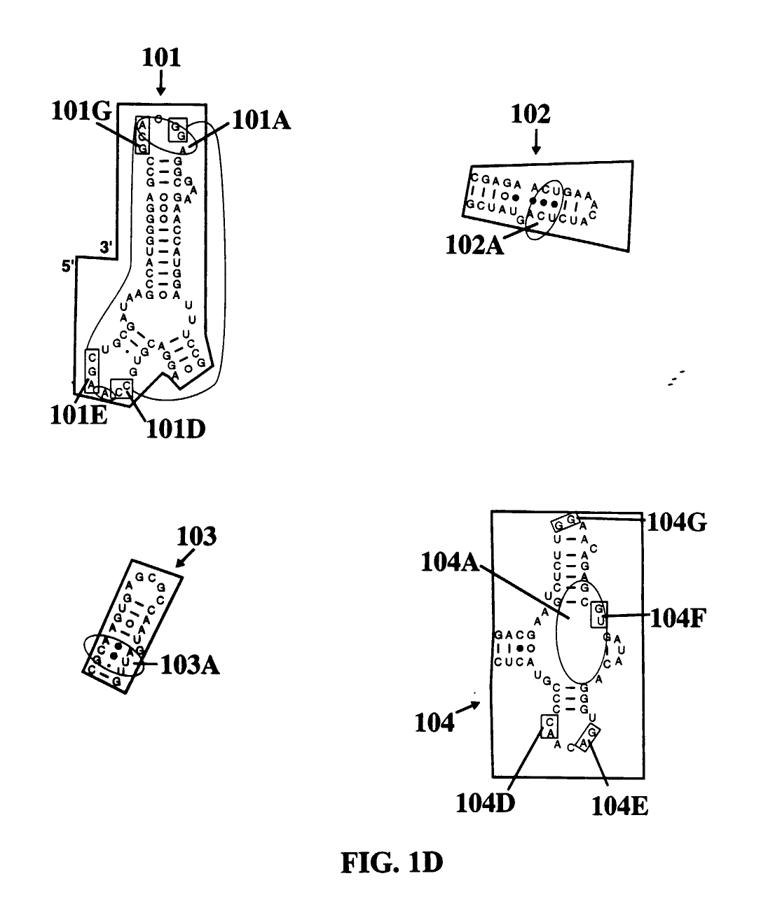

Numerous molecular interaction sites have been discovered within the consensus sequence of 23S rRNA. In the particular examples disclosed herein below, “n” refers to any nucleotide. Consensus site 1, shown in FIG. 1A as region 101, comprises a region of RNA comprising from about thirty five nucleotides to about ninety nine nucleotides, portions of which form a double-stranded RNA having the following features (5′ to 3′): a first side of a first stem comprising from about two nucleotides to about six nucleotides, a first side of a second stem comprising from about two nucleotides to about five nucleotides, a first terminal loop comprising from about four nucleotides to about twelve nucleotides, a second side of the second stem comprising from about two nucleotides to about five nucleotides, a first side of a first internal loop comprising from about three nucleotides to about seven nucleotides, a first side of a third stem comprising from about five nucleotides to about fifteen nucleotides wherein a first side of a second internal loop comprising from about one nucleotide to about three nucleotides is present in the first side of the third stem, a second terminal loop comprising from about four nucleotides to about ten nucleotides, a second side of the third stem comprising from about five nucleotides to about fifteen nucleotides wherein a second side of a second internal loop comprising from about three nucleotides to about nine nucleotides is present in the second side of the third stem, a second side of the first internal loop comprising from about two nucleotides to about five nucleotides, and a second side of the first stem comprising from about two nucleotides to about six nucleotides. As shown in FIG. 1B, nucleotides within region 101A form a pocket. Nucleotides within region 101B form an interaction (101C) with nucleotides within region 101D. Nucleotides within region 101E form an interaction (101F) with nucleotides within region 101G.

In some embodiments, consensus site 1 comprises sixty four or sixty five nucleotides, wherein portions thereof form a double-stranded RNA having the following features (5′ to 3′): a first side of a first stem comprising four nucleotides, a first side of a second stem comprising three nucleotides, a first terminal loop comprising eight nucleotides, a second side of the second stem comprising three nucleotides, a first side of a first internal loop comprising five nucleotides, a first side of a third stem comprising ten nucleotides wherein a first side of a second internal loop comprising two nucleotides is present between the seventh and eighth nucleotides of the first side of the third stem, a second terminal loop comprising seven nucleotides, a second side of the third stem comprising ten nucleotides wherein a second side of a second internal loop comprising five or six nucleotides is present between the third and fourth nucleotides of the second side of the third stem, a second side of the first internal loop comprising three nucleotides, and a second side of the first stem comprising four nucleotides. In some embodiments, the polynucleotide comprises the sequence 5′-aggangnnnnnnncnncnnnanncnnnggnnagnngnnnnnnnncnnn nanccnnngnunuccg-3′ (SEQ ID NO:1) or 5′-aggangnnnnnnncnncnnnanncnnnggnnagn ngnnnnnnnncnnnnnanccnnngnunuccg-3′ (SEQ ID NO:2). In other embodiments, the polynucleotide comprises the sequence 5′-aggacgugccaagcugcgauaagccauggggagccg cacggaggcgaagaaccauggauuuccg-3′ (SEQ ID NO:168), as shown in FIG. 1D.

Consensus site 2, shown in FIG. 1A as region 102, comprises a region of RNA comprising from about fourteen nucleotides to about thirty six nucleotides, wherein portions thereof form a double-stranded RNA having the following features (5′ to 3′): a first side of a first stem comprising from about three nucleotides to about seven nucleotides wherein a first side of an internal loop comprising from about three nucleotides to about seven nucleotides is present in the first side of the stem, a terminal loop comprising from about two nucleotides to about six nucleotides, and a second side of the stem comprising from about three nucleotides to about seven nucleotides wherein a second side of the internal loop comprising from about three nucleotides to about nine nucleotides is present in the second side of the stem. As shown in FIG. 1B, nucleotides within region 102A form a pocket.

In some embodiments, consensus site 2 comprises twenty five nucleotides, wherein portions thereof form a double-stranded RNA having the following features (5′ to 3′): a first side of a first stem comprising five nucleotides wherein a first side of an internal loop comprising five nucleotides is present between the third and fourth nucleotides of the first side of the stem, a terminal loop comprising four nucleotides, and a second side of the stem comprising five nucleotides wherein a second side of the internal loop comprising six nucleotides is present between the second and third nucleotides of the second side of the stem. In some embodiments, the polynucleotide comprises the sequence 5′-nnngaanugaaacaucunaguannn-3′ (SEQ ID NO:3). In other embodiments, the polynucleotide comprises the sequence 5′-cgagaacugaaacaucucagu aucg-3′ (SEQ ID NO:169), as shown in FIG. 1D.

Consensus site 3, shown in FIG. 1A as region 103, comprises a region of RNA comprising from about twelve nucleotides to about thirty one nucleotides, wherein portions thereof form a double-stranded RNA having the following features (5′ to 3′): a first side of a stem comprising from about two nucleotides to about six nucleotides wherein a first side of an internal loop comprising from about three nucleotides to about seven nucleotides is present in the first side of the stem, a terminal loop comprising from about three nucleotides to about seven nucleotides, and a second side of the stem comprising from about two nucleotides to about six nucleotides wherein a second side of the internal loop comprising from about two nucleotides to about five or four nucleotides is present in the second side of the stem. As shown in FIG. 1B, nucleotides within region 103A form a pocket.

In some embodiments, consensus site 3 comprises twenty one or twenty two nucleotides, wherein portions thereof form a double-stranded RNA having the following features (5′ to 3′): a first side of a stem comprising four nucleotides wherein a. first side of an internal loop comprising five nucleotides is present between the second and third nucleotides of the first side of the stem, a terminal loop comprising five nucleotides, and a second side of the stem comprising four nucleotides wherein a second side of the internal loop comprising three or four nucleotides is present between the second and third nucleotides of the second side of the stem. In some embodiments, the polynucleotide comprises the sequence 5′-nnnnguagnggcgagcgaann-3′ (SEQ ID NO:4) or 5′-nnnnguagnggcgagcgaannn-3′ (SEQ ID NO:5). In other embodiments, the polynucleotide comprises the sequence 5′-guuaguaaccgcgagugaacgc-3′ (SEQ ID NO: 170), as shown in FIG. 1D.

Consensus site 4, shown in FIG. 1A as region 104, comprises a region of RNA comprising from about thirty one nucleotides to about seventy seven nucleotides, wherein portions thereof form a double-stranded RNA having the following features (5′ to 3′): a first side of a first stem comprising from about two nucleotides to about five nucleotides, a first side of a first internal loop comprising from about two nucleotides to about five nucleotides, a first side of a second stem comprising from about three nucleotides to about seven nucleotides, a first terminal loop comprising from about three nucleotides to about nine nucleotides, a second side of the second stem comprising from about three nucleotides to about seven nucleotides, a second side of the first internal loop comprising from about one nucleotide to about three nucleotides, a first side of a third stem comprising from about one nucleotide to about two nucleotides, a second terminal loop comprising from about two nucleotides to about five nucleotides, a second side of the third stem comprising from about one nucleotide to about two nucleotides, a first side of a second internal loop comprising from about one nucleotide to about two nucleotides, a first side of a fourth stem comprising from about two nucleotides to about five nucleotides, a third terminal loop comprising from about four nucleotides to about ten nucleotides, a second side of the fourth stem comprising from about two nucleotides to about five nucleotides, a second side of the second internal loop comprising from about two nucleotides to about five nucleotides, and a second side of the first stem comprising from about two nucleotides to about five nucleotides. As shown in FIG. 1B, nucleotides within region 104A form a pocket. Nucleotides within region 104B form an interaction (104C) with nucleotides within region 104D.

In some embodiments, consensus site 4 comprises forty nine nucleotides, wherein portions thereof form a double-stranded RNA having the following features (5′ to 3′): a first side of a first stem comprising three nucleotides, a first side of a first internal loop comprising three nucleotides, a first side of a second stem comprising five nucleotides, a first terminal loop comprising six nucleotides, a second side of the second stem comprising five nucleotides, a second side of the first internal loop comprising two nucleotides, a first side of a third stem comprising one nucleotide, a second terminal loop comprising three nucleotides, a second side of the third stem comprising one nucleotide, a first side of a second internal loop comprising one nucleotide, a first side of a fourth stem comprising three nucleotides, a third terminal loop comprising seven nucleotides, a second side of the fourth stem comprising three nucleotides, a second side of the second internal loop comprising three nucleotides, and a second side of the first stem comprising three nucleotides. In some embodiments, the polynucleotide comprises the sequence 5′-nnngaannnnnuggnaagnnn nnnnnnannnggunanannccnguannn-3′ (SEQ ID NO:6). In other embodiments, the polynucleotide comprises the sequence 5′-gacgaagucucuuggaacagagcgugauacaggguga caaccccguacuc-3′ (SEQ ID NO:171), as shown in FIG. 1D.

Consensus site 5, shown in FIG. 1A as region 105, comprises a region of RNA comprising from about eight nucleotides to about twenty two nucleotides, wherein portions thereof form a double-stranded RNA having the following features (5′ to 3′): a first side of a stem comprising from about two nucleotides to about five nucleotides, a terminal loop comprising from about four nucleotides to about twelve nucleotides, and a second side of the stem comprising from about two nucleotides to about five nucleotides.

In some embodiments, consensus site 5 comprises fifteen nucleotides, wherein portions thereof form a double-stranded RNA having the following features (5′ to 3′): a first side of a stem comprising three nucleotides, a terminal loop comprising eight nucleotides, and a second side of the stem comprising three nucleotides. In some embodiments, the polynucleotide comprises the sequence 5′-nnncncgngnnannn-3′ (SEQ ID NO:7).

Consensus site 6, shown in FIG. 1A as region 106, comprises a region of RNA comprising from about ten nucleotides to about twenty six nucleotides, wherein portions thereof form a double-stranded RNA having the following features (5′ to 3′): a dangling region comprising from about one nucleotide to about three nucleotides, a first side of a stem comprising from about three nucleotides to about seven nucleotides, a terminal loop comprising from about three nucleotides to about nine nucleotides, and a second side of the stem comprising from about three nucleotides to about seven nucleotides. As shown in FIG. 1C, nucleotides within region 106A form a pocket.

In some embodiments, consensus site 6 comprises eighteen nucleotides, wherein portions thereof form a double-stranded RNA having the following features (5′ to 3′): a dangling region comprising two nucleotides, a first side of a stem comprising five nucleotides, a terminal loop comprising six nucleotides, and a second side of the stem comprising five nucleotides. In some embodiments, the polynucleotide comprises the sequence 5′-nngnnnngaccannnnnn-3′ (SEQ ID NO:8).

Consensus site 7, shown in FIG. 1A as region 107, comprises a region of RNA comprising a first and second polynucleotide. The first polynucleotide comprises from about fifteen nucleotides to about forty eight nucleotides, wherein portions of the polynucleotide form a double-stranded RNA having the following features (5′ to 3′): a first side of a first stem comprising from about one nucleotide to about three nucleotides, a first side of an internal loop comprising from about seven nucleotides to about twenty four nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about two nucleotides to about six nucleotides, and a first side of a third stem comprising from about one nucleotide to about three nucleotides. The second polynucleotide comprises from about three nucleotides to about nine nucleotides and interacts with the first polynucleotide such that the two most 5′ nucleotides form the second side of the third stem and the two most 3′ nucleotides form the second side of the first stem.

In some embodiments, the first polynucleotide of consensus site 7 comprises from thirty to thirty two nucleotides, wherein portions of the polynucleotide form a double-stranded RNA having the following features (5′ to 3′): a first side of a first stem comprising two nucleotides, a first side of an internal loop comprising from fourteen to sixteen nucleotides, a first side of a second stem comprising two nucleotides, a terminal loop comprising four nucleotides, a second side of the second stem comprising two nucleotides, a second side of the internal loop comprising four nucleotides, and a first side of a third stem comprising two nucleotides. In some embodiments, the first polynucleotide comprises the sequence 5′-ccnauagngnanaguac nguganggaaagg-3′ (SEQ ID NO:9) or 5′-ccnauagngnannaguacnguganggaaagg-3′ (SEQ ID NO:10) or 5′-ccnauagngnannnaguacnguganggaaagg-3′ (SEQ ID NO:11). In some embodiments, the second polynucleotide comprises six nucleotides and interacts with the first polynucleotide such that the two most 5′ nucleotides form the second side of the third stem and the two most 3′ nucleotides form the second side of the first stem. In some embodiments, the second polynucleotide comprises the sequence 5′-ccungg-3′.

Consensus site 8, shown in FIG. 1A as region 108, comprises a region of RNA comprising from about eighteen nucleotides to about forty nine nucleotides, wherein portions thereof form a double-stranded RNA having the following features (5′ to 3′): a first dangling region comprising from about five nucleotides to about thirteen nucleotides, a first side of a stem comprising from about two nucleotides to about six nucleotides, a terminal loop comprising from about two nucleotides to about six or five nucleotides, a second side of the stem comprising from about two nucleotides to about six nucleotides, and a second dangling region comprising from about seven nucleotides to about nineteen nucleotides. As shown in FIG. 1C, nucleotides within region 108A form a pocket.

In some embodiments, consensus site 8 comprises thirty four or thirty five nucleotides, wherein portions thereof form a double-stranded RNA having the following features (5′ to 3′): a first dangling region comprising nine nucleotides, a first side of a stem comprising four nucleotides, a terminal loop comprising four or five nucleotides, a second side of the stem comprising four nucleotides, and a second dangling region comprising thirteen nucleotides. In some embodiments, the polynucleotide comprises the sequence 5′-ngaaaagnacccnnnnangggagugaaanagnnc-3′ (SEQ ID NO:12) or 5′-ngaaaagnacccnnnnnangggagugaaanagnnc-3′ (SEQ ID NO:13).

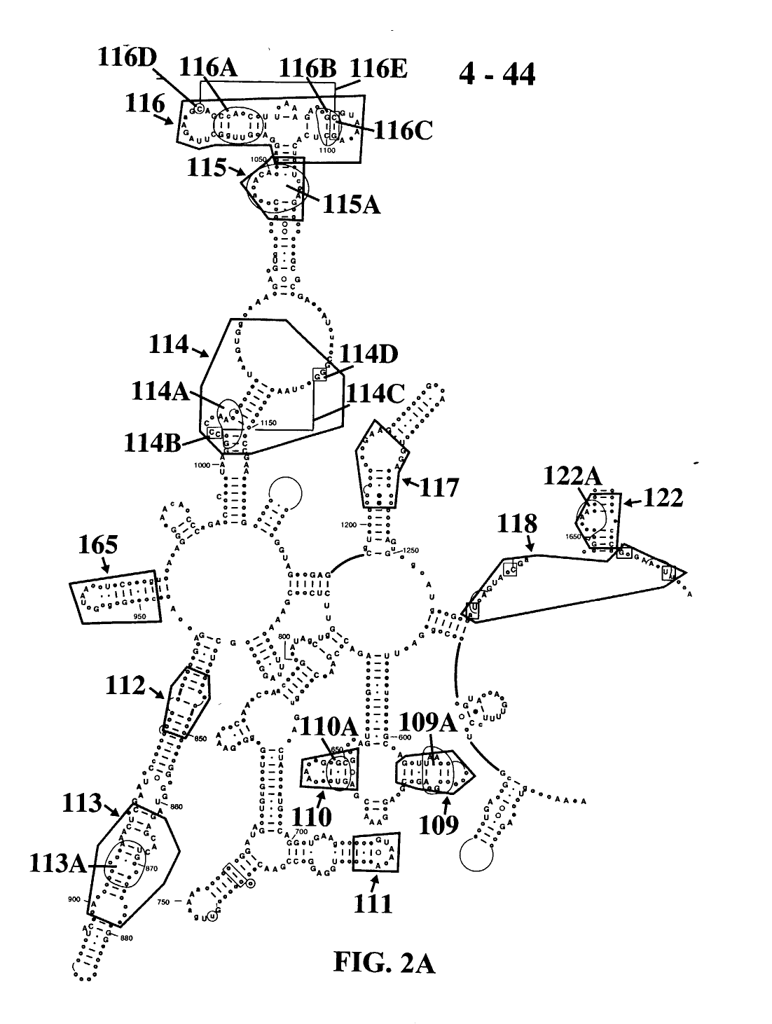

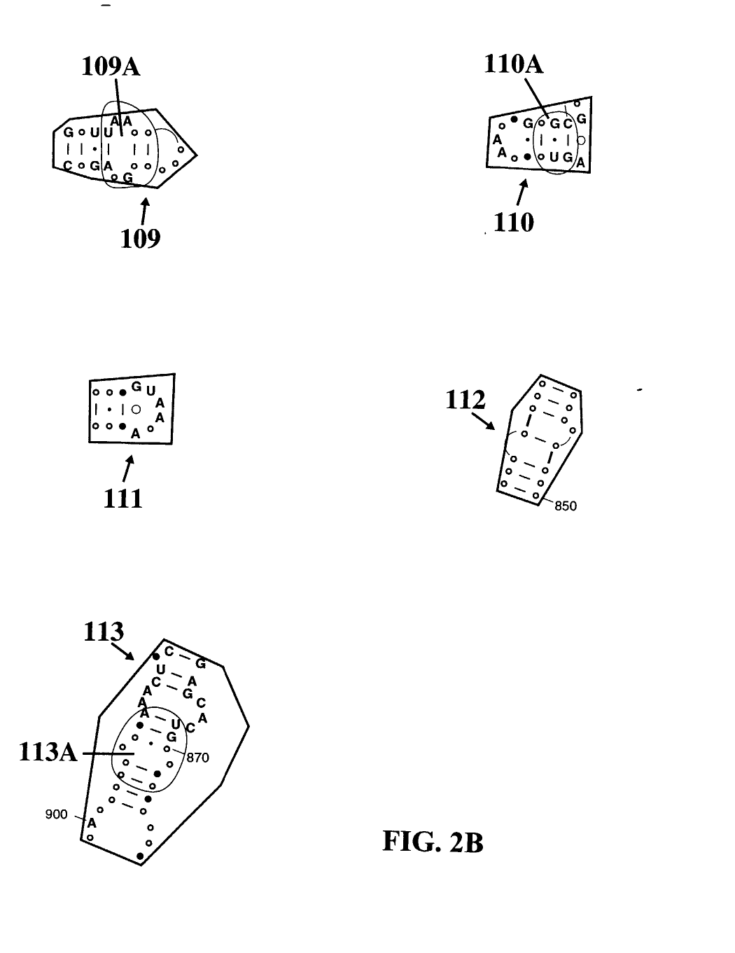

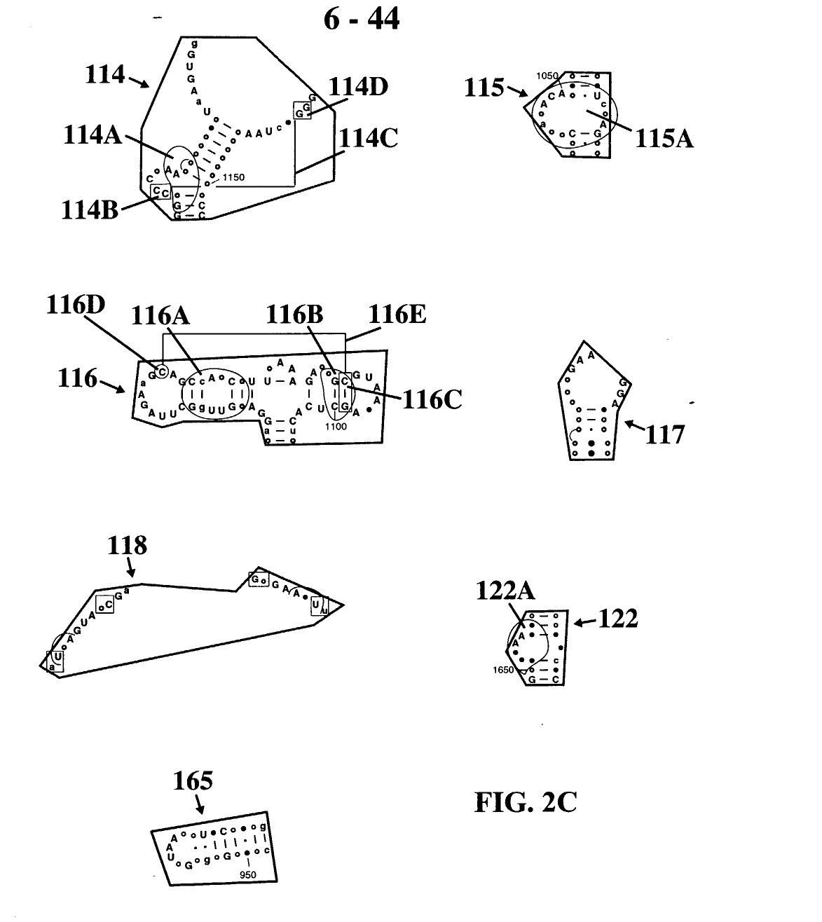

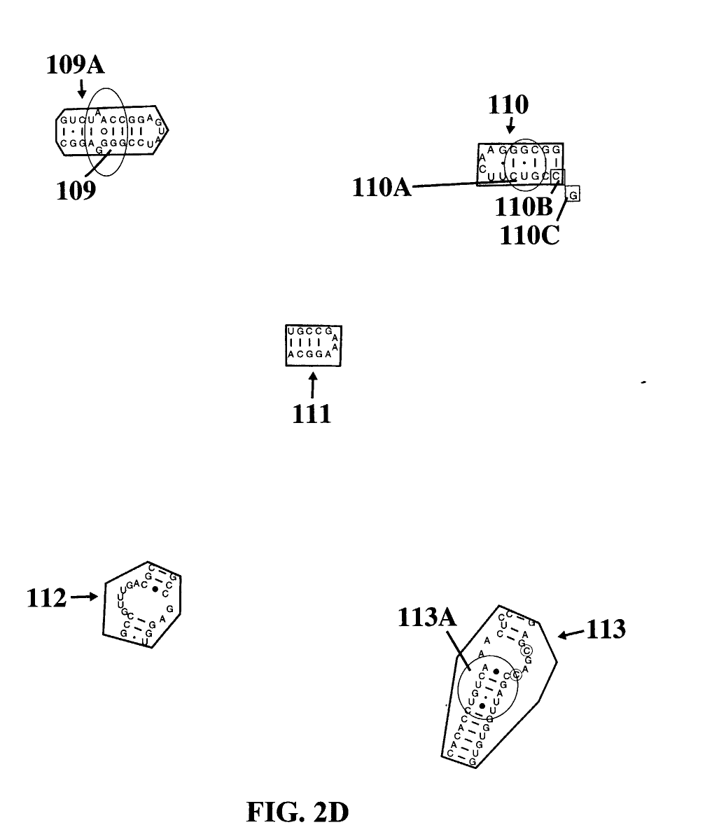

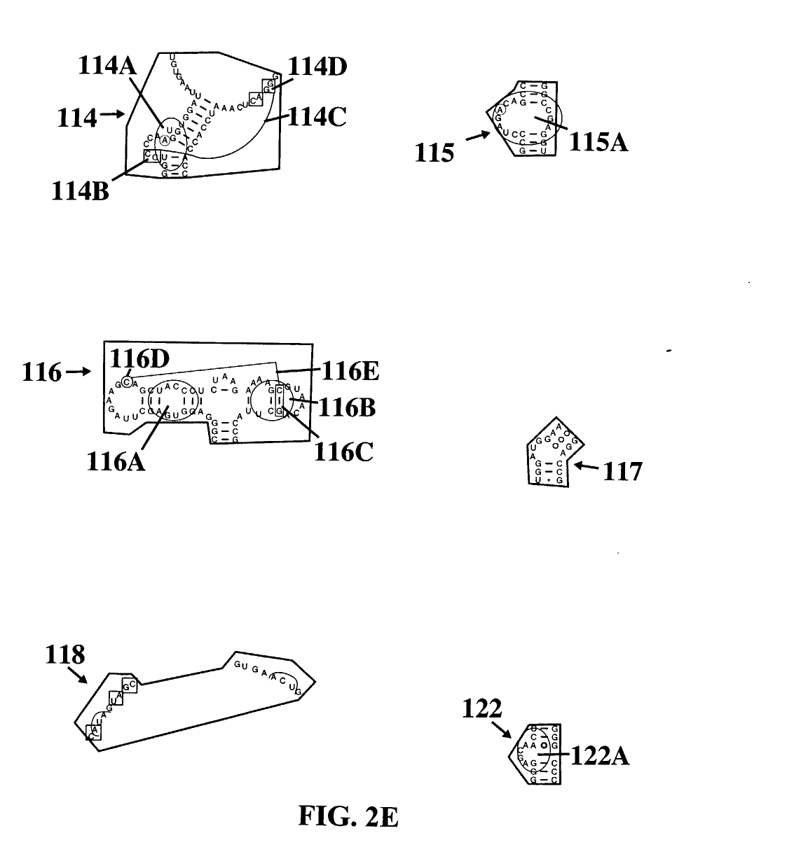

Consensus site 9, shown in FIG. 2A as region 109, comprises a region of RNA comprising from about ten nucleotides to about thirty six nucleotides, wherein portions thereof form a double-stranded RNA having the following features (5′ to 3′): a first side of a first stem comprising from about two nucleotides to about six nucleotides, a first side of an internal loop comprising from about one nucleotide to about three nucleotides, a first side of a second stem comprising from about one nucleotide to about three nucleotides, a terminal loop comprising from about two nucleotides to about twelve nucleotides, a second side of the second stem comprising from about one nucleotide to about three nucleotides, a second side of the internal loop comprising from about one nucleotide to about three nucleotides, and a second side of the first stem comprising from about two nucleotides to about six nucleotides. As shown in FIG. 2B, nucleotides within region 109A form a pocket.

In some embodiments, consensus site 9 preferably comprises twenty to twenty four nucleotides, wherein portions of the polynucleotide form a double-stranded RNA having the following features (5′ to 3′): a first side of a first stem comprising four nucleotides, a first side of an internal loop comprising two nucleotides, a first side of a second stem comprising two nucleotides, a terminal loop comprising from four to eight nucleotides, a second side of the second stem comprising two nucleotides, a second side of the internal loop comprising two nucleotides, and a second side of the first stem comprising four nucleotides. In some embodiments, the polynucleotide comprises the sequence 5′-gnuuaannnnnnnngaagnc-3′ (SEQ ID NO:14) or 5′-gnuuaannnnnnnnngaagnc-3′ (SEQ ID NO:15) or 5′-gnuuaannnnnnnnnngaagnc-3′ (SEQ ID NO:16) or 5′-gnuuaannnnnnnnnnngaagnc-3′ (SEQ ID NO:17) or 5′-gnuuaannnnnnnnnnnngaagnc-3′ (SEQ ID NO:18). In other embodiments, the polynucleotide comprises the sequence 5′-gucuaaccggaguauccgggg aggc-3′ (SEQ ID NO:172), as shown in FIG. 2D.

Consensus site 10, shown in FIG. 2A as region 110, comprises a region of RNA comprising from about nine nucleotides to about twenty three nucleotides, wherein portions thereof form a double-stranded RNA having the following features (5′ to 3′): a dangling region comprising from about one nucleotide to about two nucleotides, a first side of a stem comprising from about two nucleotides to about six nucleotides, a terminal loop comprising from about three nucleotides to about seven nucleotides, a second side of the stem comprising from about two nucleotides to about six nucleotides, and a dangling region comprising from about one nucleotide to about two nucleotides. As shown in FIG. 2B, nucleotides within region 110A form a pocket.

In some embodiments, consensus site 10 comprises sixteen nucleotides, wherein portions thereof form a double-stranded RNA having the following features (5′ to 3′): a dangling region comprising one nucleotide, a first side of a stem comprising four nucleotides, a terminal loop comprising five nucleotides, a second side of the stem comprising four nucleotides, and a dangling region comprising one nucleotide. In some embodiments, the polynucleotide comprises the sequence 5′-agunnnaanngngcg-3′ (SEQ ID NO:19). In other embodiments, the polynucleotide comprises the sequence 5′-gccgucuucaagggcgg-3′ (SEQ ID NO:173), as shown in FIG. 2D.

Consensus site 11, shown in FIG. 2A as region 111, comprises a region of RNA comprising from about seven nucleotides to about nineteen nucleotides, wherein portions thereof form a double-stranded RNA having the following features (5′ to 3′): a first side of a stem comprising from about two nucleotides to about five nucleotides, a terminal loop comprising from about three nucleotides to about nine nucleotides, and a second side of the stem comprising from about two nucleotides to about five nucleotides.

In some embodiments, consensus site 11 comprises twelve nucleotides, wherein portions thereof form a double-stranded RNA having the following features (5′ to 3′): a first side of a stem comprising three nucleotides, a terminal loop comprising six nucleotides, and a second side of the stem comprising three nucleotides. In some embodiments, the polynucleotide comprises the sequence 5′-nnnguaanannn-3′ (SEQ ID NO:20). In other embodiments, the polynucleotide comprises the sequence 5′-ugccgaaaggca-3′ (SEQ ID NO:174), as shown in FIG. 2D.

Consensus site 12, shown in FIG. 2A as region 112, comprises a region of RNA comprising a first and second polynucleotide. The first polynucleotide comprises from about five nucleotides to about fourteen nucleotides and comprises the following features (5′ to 3′): a first side of a stem comprising from about four nucleotides to about ten nucleotides wherein a first bulge comprising from about one nucleotides to about two to four nucleotides is present in the first side of the stem. The second polynucleotide comprises from about five nucleotides to about sixteen nucleotides and comprises the following features (5′ to 3′): a second side of the stem comprising from about four nucleotides to about ten nucleotides wherein a second bulge comprising from about one nucleotide to about six nucleotides is optionally present in the second side of the stem.

In some embodiments, the first polynucleotide of consensus site 12 comprises from eight to eleven nucleotides and comprises the following features (5′ to 3′): a first side of a stem comprising seven nucleotides wherein a first bulge comprising one to four nucleotides is present between the third and fourth nucleotides of the first side of the stem. In some embodiments, the first polynucleotide comprises the sequence 5′-nnnnnnnn-3′ or 5′-nnnnnnnnn-3′ or 5′-nnnnnnnnnn-3′ (SEQ ID NO:21) or 5′-nnnnnnnnnnn-3′ (SEQ ID NO:22). In other embodiments, the first polynucleotide comprises the sequence 5′-gccgaggu-3′, as shown in FIG. 2D. In some embodiments, the second polynucleotide comprises seven to twelve nucleotides and comprises the following features (5′ to 3′): a second side of the stem comprising seven nucleotides wherein a second bulge comprising one to four nucleotides is optionally present between the third and fourth nucleotides of the second side of the stem. In some embodiments, the second polynucleotide comprises the sequence 5′-nnnnnnn-3′ or 5′-nnnnnnnn-3′ or 5′-nnnnnnnnn-3′ or 5-nnnnnnnnnn-3′ (SEQ ID NO:23) or 5′-nnnnnnnnnnn-3′ (SEQ ID NO:24) or 5′-nnnnnnnnnnnn-3′ (SEQ ID NO:25). In other embodiments, the second polynucleotide comprises the sequence 5′-gccguuugacgc-3′ (SEQ ID NO:175), as shown in FIG. 2D.

Consensus site 13, shown in FIG. 2A as region 113, comprises a region of RNA comprising a first and second polynucleotide. The first polynucleotide comprises from about eleven nucleotides to about twenty eight nucleotides and comprises the following features (5′ to 3′): a first side of a first stem comprising from about two nucleotides to about five nucleotides, a first side of a first internal loop comprising from about two nucleotides to about five nucleotides, a first side of a second stem comprising from about two nucleotides to about five nucleotides, a first side of a second internal loop comprising from about one nucleotide to about two nucleotides, a first side of a third stem comprising from about two nucleotides to about six nucleotides, and a dangling region comprising from about two nucleotides to about five nucleotides. The second polynucleotide comprises from about twelve nucleotides to about twenty eight nucleotides and comprises the following features (5′ to 3′): a dangling region comprising from about two nucleotides to about five nucleotides, a second side of the third stem comprising from about two nucleotides to about six nucleotides, a second side of the second internal loop comprising from about one nucleotide to about two nucleotides, a second side of the second stem comprising from about two nucleotides to about five nucleotides, a second side of the first internal loop comprising from about one nucleotide to about three nucleotides, and a second side of the first stem comprising from about two nucleotides to about five nucleotides wherein a bulge comprising from about one nucleotide to about two nucleotides is present in the second side of the first stem. As shown in FIG. 2B, nucleotides within region 113A form a pocket.

In some embodiments, the first polynucleotide of consensus site 13 comprises seventeen nucleotides and comprises the following features (5′ to 3′): a first side of a first stem comprising three nucleotides, a first side of a first internal loop comprising three nucleotides, a first side of a second stem comprising three nucleotides, a first side of a second internal loop comprising one nucleotide, a first side of a third stem comprising four nucleotides, and a dangling region comprising three nucleotides. In some embodiments, the first polynucleotide comprises the sequence 5′-gagcacugnnnnnnnnn-3′ (SEQ ID NO:26). In other embodiments, the polynucleotide comprises the sequence 5′-gagcgaccgauuggugug-3′ (SEQ ID NO:176), as shown in FIG. 2D. In some embodiments, the second polynucleotide comprises seventeen nucleotides and comprises the following features (5′ to 3′): a dangling region comprising three nucleotides, a second side of the third stem comprising four nucleotides, a second side of the second internal loop comprising one nucleotide, a second side of the second stem comprising three nucleotides, a second side of the first internal loop comprising two nucleotides, and a second side of the first stem comprising three nucleotides wherein a bulge comprising one nucleotide is present between the second and third nucleotides of the second side of the first stem. In some embodiments, the second polynucleotide comprises the sequence 5′ -nannnnnnnnaaacunc-3′ (SEQ ID NO:27). In other embodiments, the polynucleotide comprises the sequence 5′-cacaccugucaaacucc-3′ (SEQ ID NO:177), as shown in FIG. 2D.

Consensus site 14, shown in FIG. 2A as region 114, comprises a region of RNA comprising a first and second polynucleotide. The first polynucleotide comprises from about fourteen nucleotides to about thirty six nucleotides and comprises the following features (5′ to 3′): a first side of a first stem comprising from about two nucleotides to about five nucleotides, a bulge comprising from about three nucleotides to about nine nucleotides, a first side of a second stem comprising from about four nucleotides to about ten nucleotides wherein a bulge of from about one nucleotide to about two nucleotides is optionally present in the first side of the first stem, and a dangling region comprising from about four nucleotides to about ten nucleotides. The second polynucleotide comprises from about ten nucleotides to about twenty seven nucleotides and comprises the following features (5′ to 3′): a dangling region comprising from about four nucleotides to about twelve nucleotides, a second side of the second stem comprising from about four nucleotides to about ten nucleotides, and a second side of the first stem comprising from about two nucleotides to about five nucleotides. As shown in FIG. 2C, nucleotides within region 114A form a pocket. Nucleotides within region 114B form an interaction (114C) with nucleotides within region 114D.

In some embodiments, the first polynucleotide of consensus site 14 comprises twenty three or twenty four nucleotides and comprises the following features (5′ to 3′): a first side of a first stem comprising three nucleotides, a bulge comprising six nucleotides, a first side of a second stem comprising seven nucleotides wherein a bulge of one nucleotide is optionally present between the first and second nucleotides of the first side of the first stem, and a dangling region comprising seven nucleotides. In some embodiments, the first polynucleotide comprises the sequence 5′-ggncccnaannnnnnnuaagugg-3′ (SEQ ID NO:28) or 5′-ggncccnaannnnnnnnuaag ugg-3′ (SEQ ID NO:29). In other embodiments, the first polynucleotide comprises the sequence 5′-gguccccaaguguggauuaagugu-3′ (SEQ ID NO:178), as shown in FIG. 2E. In some embodiments, the second polynucleotide comprises eighteen nucleotides and comprises the following features (5′ to 3′): a dangling region comprising eight nucleotides, a second side of the second stem comprising seven nucleotides, and a second side of the first stem comprising three nucleotides. In some embodiments, the second polynucleotide comprises the sequence 5′-gggncuaannnnnnnncc-3′ (SEQ ID NO:30). In other embodiments, the polynucleotide comprises the sequence 5′-gggacucaaauccaccacc-3′ (SEQ ID NO:179), as shown in FIG. 2E.

Consensus site 15, shown in FIG. 2A as region 115, comprises a region of RNA comprising a first and second polynucleotide. The first polynucleotide comprises from about eight nucleotides to about twenty nucleotides and comprises the following features (5′ to 3′): a first side of a first stem comprising from about two nucleotides to about five nucleotides, a first side of an internal loop comprising from about four nucleotides to about ten nucleotides, and a first side of a second stem comprising from about two nucleotides to about five nucleotides. The second polynucleotide comprises from about six nucleotides to about fifteen nucleotides and comprises the following features (5′ to 3′): a second side of the second stem comprising from about two nucleotides to about five nucleotides, a second side of the internal loop comprising from about two nucleotides to about five nucleotides, and a second side of the first stem comprising from about two nucleotides to about five nucleotides. As shown in FIG. 2C, nucleotides within region 115A form a pocket.

In some embodiments, the first polynucleotide of consensus site 15 comprises thirteen nucleotides and comprises the following features (5′ to 3′): a first side of a first stem comprising three nucleotides, a first side of an internal loop comprising seven nucleotides, and a first side of a second stem comprising three nucleotides. In some embodiments, the first polynucleotide comprises the sequence 5′-nncnnanacannn-3′ (SEQ ID NO:31). In other embodiments, the first polynucleotide comprises the sequence 5′-gcccuagacagcc-3′ (SEQ ID NO:180), as shown in FIG. 2E. In some embodiments, the second polynucleotide comprises nine nucleotides and comprises the following features (5′ to 3′): a second side of the second stem comprising three nucleotides, a second side of the internal loop comprising three nucleotides, and a second side of the first stem comprising three nucleotides. In some embodiments, the second polynucleotide comprises the sequence 5′-nnucnagnn-3′. In other embodiments, the second polynucleotide comprises the sequence 5′-ggccgaggu-3′ (SEQ I) NO:181), as shown in FIG. 2E.

Consensus site 16, shown in FIG. 2A as region 116, comprises a region of RNA comprising from about thirty five nucleotides to about eighty eight nucleotides, wherein portions thereof form a double-stranded RNA having the following features (5′ to 3′): a first side of a first stem comprising from about two nucleotides to about five nucleotides, a first bulge of from about one nucleotide to about two nucleotides, a first side of a second stem comprising from about two nucleotides to about five nucleotides, a first side of a first internal loop comprising from about one nucleotide to about three nucleotides, a first side of a third stem comprising from about two nucleotides to about five nucleotides, a first terminal loop comprising from about five nucleotides to about thirteen nucleotides, a second side of the third stem comprising from about two nucleotides to about five nucleotides, a second side of the first internal loop comprising from about one nucleotide to about three nucleotides, a second side of the second stem comprising from about two nucleotides to about five nucleotides, a first side of a fourth stem comprising from about one nucleotide to about two nucleotides, a second terminal loop comprising from about two nucleotides to about five nucleotides, a second side of the fourth stem comprising from about one nucleotide to about two nucleotides, a first side of a fifth stem comprising from about two nucleotides to about five nucleotide wherein a first side of a second internal loop comprising from about two nucleotides to about five nucleotides is present in the first side of the fifth stem, a third terminal loop comprising from about three nucleotides to about nine nucleotides, a second side of the fifth stem comprising from about two nucleotides to about five nucleotides wherein a second side of the second internal loop comprising from about one nucleotide to about two nucleotides is present in the second side of the fifth stem, a second bulge of from about one nucleotide to about two nucleotides, and a second side of the first stem comprising from about two nucleotides to about five nucleotides. As shown in FIG. 2C, nucleotides within regions 116A and 116B form pockets. The nucleotide within region 116D forms an interaction (116E) with nucleotides within region 116C.

In some embodiments, consensus site 16 comprises fifty four nucleotides, wherein portions thereof form a double-stranded RNA having the following features (5′ to 3′): a first side of a first stem comprising three nucleotides, a first bulge of one nucleotide, a first side of a second stem comprising three nucleotides, a first side of a first internal loop comprising two nucleotides, a first side of a third stem comprising three nucleotides, a first terminal loop comprising nine nucleotides, a second side of the third stem comprising three nucleotides, a second side of the first internal loop comprising two nucleotides, a second side of the second stem comprising three nucleotides, a first side of a fourth stem comprising one nucleotide, a second terminal loop comprising three nucleotides, a second side of the fourth stem comprising one nucleotide, a first side of a fifth stem comprising three nucleotide wherein a first side of a second internal loop comprising three nucleotides is present between the first and second nucleotides of the first side of the fifth stem, a third terminal loop comprising six nucleotides, a second side of the fifth stem comprising three nucleotides wherein a second side of the second internal loop comprising one nucleotide is present between the second and third nucleotides of the second side of the fifth stem, a second bulge of one nucleotide, and a second side of the first stem comprising three nucleotides. In some embodiments, the polynucleotide comprises the sequence 5′ -nagganguuggcuuagaagcagccancnuunaaaganngcguaanagcucacun-3′ (SEQ ID NO:32). In other embodiments, the polynucleotide comprises the sequence 5′-cgggaggugagcuu agaagcagcuacccucuaagaaaagcguaacagcuuaccg-3′ (SEQ ID NO:182), as shown in FIG. 2E. The molecular interaction site comprises a drug-binding pocket encompassing an area defined by about 12Å by 8Å (the region formed by the interaction of regions 116A and 116B) and is enclosed within a deep pocket formed by the major grooves of stems 43 and 44 facing each other.

Consensus site 17, shown in FIG. 2A as region 117, comprises a region of RNA comprising a first and second polynucleotide. The first polynucleotide comprises from about seven nucleotides to about one hundred sixty six nucleotides and comprises the following features (5′ to 3′): a first side of a stem comprising from about three nucleotides to about seven nucleotides wherein a bulge comprising from about one nucleotide to about one hundred fifty nucleotides is optionally present in the first side of the stem, and a dangling region comprising from about three nucleotides to about nine nucleotides. The second polynucleotide comprises from about five nucleotides to about twelve nucleotides and comprises the following features (5′ to 3′): a dangling region comprising from about two nucleotides to about five nucleotides and a second side of the stem comprising from about three nucleotides to about seven nucleotides.

In some embodiments, the first polynucleotide of consensus site 17 comprises from eleven to one hundred fourteen nucleotides and comprises the following features (5′ to 3′): a first side of a stem comprising five nucleotides wherein a bulge comprising from one to one hundred three nucleotides is optionally present between the second and third nucleotides of the first side of the stem, and a dangling region comprising six nucleotides. In some embodiments, the first polynucleotide comprises the sequence

| | |
|---|---|
| 5′-nnnnnnnngaa-3′, | (SEQ ID NO:33) |
| 5′-nnnnnnnnngaa-3′, | (SEQ ID NO:34) |
| 5′-nnnnnnnnnngaa-3′, | (SEQ ID NO:35) |
| 5′-nnnnnnnnnnngaa-3′, | (SEQ ID NO:36) |
| 5′-nnnnnnnnnnnngaa-3′, | (SEQ ID NO:37) |
| 5′-nnnnnnnnnnnnngaa-3′, | (SEQ ID NO:38) |
| 5′-nnnnnnnnnnnnnngaa-3′, | (SEQ ID NO:39) |
| 5′-nnnnnnnnnnnnnnngaa-3′, | (SEQ ID NO:40) |
| 5′-nnnnnnnnnnnnnnnngaa-3′, | (SEQ ID NO:41) |
| 5′-nnnnnnnnnnnnnnnnngaa-3′, | (SEQ ID NO:42) |
| 5′-nnnnnnnnnnnnnnnnnngaa-3′, | (SEQ ID NO:43) |
| 5′-nnnnnnnnnnnnnnnnnnngaa-3′, | (SEQ ID NO:44) |
| 5′-nnnnnnnnnnnnnnnnnnnngaa-3′, | (SEQ ID NO:45) |
| 5′-nnnnnnnnnnnnnnnnnnnnngaa-3′, | (SEQ ID NO:46) |
| 5′-nnnnnnnnnnnnnnnnnnnnnngaa-3′, | (SEQ ID NO:47) |
| 5′-nnnnnnnnnnnnnnnnnnnnnnngaa-3′, | (SEQ ID NO:48) |
| 5′-nnnnnnnnnnnnnnnnnnnnnnnngaa-3′, | (SEQ ID NO:49) |
| 5′-nnnnnnnnnnnnnnnnnnnnnnnnngaa-3′, | (SEQ ID NO:50) |
| 5′-nnnnnnnnnnnnnnnnnnnnnnnnnngaa-3′, | (SEQ ID NO:51) |
| 5′-nnnnnnnnnnnnnnnnnnnnnnnnnnngaa-3′, | (SEQ ID NO:52) |
| 5′-nnnnnnnnnnnnnnnnnnnnnnnnnnnngaa-3′, | (SEQ ID NO:53) |
| 5′-nnnnnnnnnnnnnnnnnnnnnnnnnnnnngaa-3′, | (SEQ ID NO:54) |
| 5′-nnnnnnnnnnnnnnnnnnnnnnnnnnnnnngaa-3′, | (SEQ ID NO:55) |
| 5′-nnnnnnnnnnnnnnnnnnnnnnnnnnnnnnngaa-3′, | (SEQ ID NO:56) |
| 5′-nnnnnnnnnnnnnnnnnnnnnnnnnnnnnnnngaa-3′, | (SEQ ID NO:57) |
| 5′-nnnnnnnnnnnnnnnnnnnnnnnnnnnnnnnnngaa-3′, | (SEQ ID NO:58) |
| 5′-nnnnnnnnnnnnnnnnnnnnnnnnnnnnnnnnnngaa-3′, | (SEQ ID NO:59) |
| 5′-nnnnnnnnnnnnnnnnnnnnnnnnnnnnnnnnnnngaa-3′, | (SEQ ID NO:60) |
| 5′-nnnnnnnnnnnnnnnnnnnnnnnnnnnnnnnnnnnngaa-3′, | (SEQ ID NO:61) |
| 5′-nnnnnnnnnnnnnnnnnnnnnnnnnnnnnnnnnnnnngaa-3′, | (SEQ ID NO:62) |
| 5′-nnnnnnnnnnnnnnnnnnnnnnnnnnnnnnnnnnnnnngaa-3′, | (SEQ ID NO:63) |
| 5′-nnnnnnnnnnnnnnnnnnnnnnnnnnnnnnnnnnnnnnngaa-3′, | (SEQ ID NO:64) |
| 5′-nnnnnnnnnnnnnnnnnnnnnnnnnnnnnnnnnnnnnnnngaa-3′, | (SEQ ID NO:65) |
| 5′-nnnnnnnnnnnnnnnnnnnnnnnnnnnnnnnnnnnnnnnnnngaa-3′, | (SEQ ID NO:66) |
| 5′-nnnnnnnnnnnnnnnnnnnnnnnnnnnnnnnnnnnnnnnnnngaa-3′, | (SEQ ID NO:67) |
| 5′-nnnnnnnnnnnnnnnnnnnnnnnnnnnnnnnnnnnnnnnnnnngaa-3′, | (SEQ ID NO:68) |
| 5′-nnnnnnnnnnnnnnnnnnnnnnnnnnnnnnnnnnnnnnnnnnnngaa-3′, | (SEQ ID NO:69) |
| 5′-nnnnnnnnnnnnnnnnnnnnnnnnnnnnnnnnnnnnnnnnnnnnngaa-3′, | (SEQ ID NO:70) |
| 5′-nnnnnnnnnnnnnnnnnnnnnnnnnnnnnnnnnnnnnnnnnnnnnngaa-3′, | (SEQ ID NO:71) |
| 5′-nnnnnnnnnnnnnnnnnnnnnnnnnnnnnnnnnnnnnnnnnnnnnnngaa-3′, | (SEQ ID NO:72) |
| 5′-nnnnnnnnnnnnnnnnnnnnnnnnnnnnnnnnnnnnnnnnnnnnnnnngaa-3′, | (SEQ ID NO:73) |
| 5′-nnnnnnnnnnnnnnnnnnnnnnnnnnnnnnnnnnnnnnnnnnnnnnnnngaa-3′, | (SEQ ID NO:74) |
| 5′-nnnnnnnnnnnnnnnnnnnnnnnnnnnnnnnnnnnnnnnnnnnnnnnnngaa-3′, | (SEQ ID NO:75) |
| 5′-nnnnnnnnnnnnnnnnnnnnnnnnnnnnnnnnnnnnnnnnnnnnnnnnnngaa-3′, | (SEQ ID NO:76) |
| 5′-nnnnnnnnnnnnnnnnnnnnnnnnnnnnnnnnnnnnnnnnnnnnnnnnnnnngaa-3′, | (SEQ ID NO:77) |
| 5′-nnnnnnnnnnnnnnnnnnnnnnnnnnnnnnnnnnnnnnnnnnnnnnnnnnnnngaa-3′, | (SEQ ID NO:78) |
| 5′-nnnnnnnnnnnnnnnnnnnnnnnnnnnnnnnnnnnnnnnnnnnnnnnnnnnnnngaa-3′, | (SEQ ID NO:79) |
| 5′-nnnnnnnnnnnnnnnnnnnnnnnnnnnnnnnnnnnnnnnnnnnnnnnnnnnnnnngaa-3′, | (SEQ ID NO:80) |
| 5′-nnnnnnnnnnnnnnnnnnnnnnnnnnnnnnnnnnnnnnnnnnnnnnnnnnnnnnnngaa-3′, | (SEQ ID NO:81) |
| 5′-nnnnnnnnnnnnnnnnnnnnnnnnnnnnnnnnnnnnnnnnnnnnnnnnnnnnnnnnngaa-3′, | (SEQ ID NO:82) |
| 5′-nnnnnnnnnnnnnnnnnnnnnnnnnnnnnnnnnnnnnnnnnnnnnnnnnnnnnnnnnngaa-3′, | (SEQ ID NO:83) |
| 5′-nnnnnnnnnnnnnnnnnnnnnnnnnnnnnnnnnnnnnnnnnnnnnnnnnnnnnnnnnnngaa-3′, | (SEQ ID NO:84) |
| 5′-nnnnnnnnnnnnnnnnnnnnnnnnnnnnnnnnnnnnnnnnnnnnnnnnnnnnnnnnnnnngaa-3′, | (SEQ ID NO:85) |
| 5′-nnnnnnnnnnnnnnnnnnnnnnnnnnnnnnnnnnnnnnnnnnnnnnnnnnnnnnnnnnnnngaa-3′, | (SEQ ID NO:86) |
| 5′-nnnnnnnnnnnnnnnnnnnnnnnnnnnnnnnnnnnnnnnnnnnnnnnnnnnnnnnnnnnnngaa-3′, | (SEQ ID NO:87) |
| 5′-nnnnnnnnnnnnnnnnnnnnnnnnnnnnnnnnnnnnnnnnnnnnnnnnnnnnnnnnnnnnnnngaa-3′, | (SEQ ID NO:88) |
| 5′-nnnnnnnnnnnnnnnnnnnnnnnnnnnnnnnnnnnnnnnnnnnnnnnnnnnnnnnnnnnnnnnngaa-3′, | (SEQ ID NO:89) |
| 5′-nnnnnnnnnnnnnnnnnnnnnnnnnnnnnnnnnnnnnnnnnnnnnnnnnnnnnnnnnnnnnnnnngaa-3′, | (SEQ ID NO:90) |
| 5′-nnnnnnnnnnnnnnnnnnnnnnnnnnnnnnnnnnnnnnnnnnnnnnnnnnnnnnnnnnnnnnnnnngaa-3′, | (SEQ ID NO:91) |
| 5′-nnnnnnnnnnnnnnnnnnnnnnnnnnnnnnnnnnnnnnnnnnnnnnnnnnnnnnnnnnnnnnnnnnnngaa-3′, | (SEQ ID NO:92) |
| 5′-nnnnnnnnnnnnnnnnnnnnnnnnnnnnnnnnnnnnnnnnnnnnnnnnnnnnnnnnnnnnnnnnnnnngaa-3′, | (SEQ ID NO:93) |
| 5′-nnnnnnnnnnnnnnnnnnnnnnnnnnnnnnnnnnnnnnnnnnnnnnnnnnnnnnnnnnnnnnnnnnnnngaa-3′, | (SEQ ID NO:94) |
| 5′-nnnnnnnnnnnnnnnnnnnnnnnnnnnnnnnnnnnnnnnnnnnnnnnnnnnnnnnnnnnnnnnnnnnnnngaa-3′, | (SEQ ID NO:95) |
| 5′-nnnnnnnnnnnnnnnnnnnnnnnnnnnnnnnnnnnnnnnnnnnnnnnnnnnnnnnnnnnnnnnnnnnnnnngaa-3′, | (SEQ ID NO:96) |
| 5′-nnnnnnnnnnnnnnnnnnnnnnnnnnnnnnnnnnnnnnnnnnnnnnnnnnnnnnnnnnnnnnnnnnnnnnnngaa-3′, | (SEQ ID NO:97) |
| 5′-nnnnnnnnnnnnnnnnnnnnnnnnnnnnnnnnnnnnnnnnnnnnnnnnnnnnnnnnnnnnnnnnnnnnnnnnngaa-3′, | (SEQ ID NO:98) |
| 5′-nnnnnnnnnnnnnnnnnnnnnnnnnnnnnnnnnnnnnnnnnnnnnnnnnnnnnnnnnnnnnnnnnnnnnnnnnngaa-3′, | (SEQ ID NO:99) |
| 5′-nnnnnnnnnnnnnnnnnnnnnnnnnnnnnnnnnnnnnnnnnnnnnnnnnnnnnnnnnnnnnnnnnnnnnnnnnnngaa- | (SEQ ID NO:100) |
| 3′, | |
| 5′-nnnnnnnnnnnnnnnnnnnnnnnnnnnnnnnnnnnnnnnnnnnnnnnnnnnnnnnnnnnnnnnnnnnnnnnnnnnngaa- | (SEQ ID NO:101) |
| 3′, | |
| 5′-nnnnnnnnnnnnnnnnnnnnnnnnnnnnnnnnnnnnnnnnnnnnnnnnnnnnnnnnnnnnnnnnnnnnnnnnnnnnngaa- | (SEQ ID NO:102) |
| 3′, | |
| 5′-nnnnnnnnnnnnnnnnnnnnnnnnnnnnnnnnnnnnnnnnnnnnnnnnnnnnnnnnnnnnnnnnnnnnnnnnnnnnnngaa- | (SEQ ID NO:103) |
| 3′, | |
| 5′-nnnnnnnnnnnnnnnnnnnnnnnnnnnnnnnnnnnnnnnnnnnnnnnnnnnnnnnnnnnnnnnnnnnnnnnnnnnnnnngaa- | (SEQ ID NO:104) |
| 3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnga | (SEQ ID NO:105) |
| a-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnga | (SEQ ID NO:106) |
| a-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnng | (SEQ ID NO:107) |
| aa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:108) |
| gaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:109) |
| ngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:110) |
| nngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:111) |
| nnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:112) |
| nnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:113) |
| nnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:114) |
| nnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:115) |
| nnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:116) |
| nnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:117) |
| nnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:118) |
| nnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:119) |
| nnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:120) |
| nnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:121) |
| nnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:122) |
| nnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:123) |
| nnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:124) |
| nnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:125) |
| nnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:126) |
| nnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:127) |
| nnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:128) |
| nnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:129) |
| nnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:130) |
| nnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:131) |
| nnnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:132) |
| nnnnnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:133) |
| nnnnnnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:134) |
| nnnnnnnnnnnnnnnnnnnnnnnnnngaa-3′, | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:135) |
| nnnnnnnnnnnnnnnnnnnnnnnnnnngaa-3′, or | |
| 5′-nnnnnnnnnnnnnnnnnnnnnnnnnnnnnnnnnnnnnnnnnnnnnnnnnnnnnnnnnnnnnnnnnnnnnnnnnnnnnnnnnnn | (SEQ ID NO:136) |
| nnnnnnnnnnnnnnnnnnnnnnnnnnnngaa-3′. | |

In other embodiments, the first polynucleotide comprises the sequence 5′-uggauggaa-3′, as shown in FIG. 2E. In some embodiments, the second polynucleotide comprises eight nucleotides and comprises the following features (5′ to 3′): a dangling region comprising three nucleotides and a second side of the stem comprising five nucleotides. In some embodiments, the second polynucleotide comprises the sequence 5′-ggannnnn-3′. In other embodiments, the second polynucleotide comprises the sequence 5′-ggaccg-3′ (SEQ ID NO:183), as shown in FIG. 2E.

Consensus site 18, shown in FIG. 2A as region 118, comprises a region of RNA comprising a first and second polynucleotide. The first polynucleotide comprises from about six nucleotides to about seventeen nucleotides and comprises the following features (5′ to 3′): a first side of a stem comprising from about four nucleotides to about twelve nucleotides wherein a bulge comprising from about one nucleotide to about two nucleotides is present in the first side of the stem, and a dangling region comprising from about one nucleotide to about three nucleotides. The second polynucleotide comprises from about eight nucleotides to about twelve nucleotides and comprises the following features (5′ to 3′): a second side of the stem comprising from about four nucleotides to about twelve nucleotides.

In some embodiments, the first polynucleotide of consensus site 18 comprises eleven nucleotides and comprises the following features (5′ to 3′): a first side of a stem comprising eight nucleotides wherein a bulge comprising one nucleotide is present between the fourth and fifth nucleotides of the first side of the stem, and a dangling region comprising two nucleotides. In some embodiments, the first polynucleotide comprises the sequence 5′-aunaguancga-3′ (SEQ ID NO:137). In other embodiments, the first polynucleotide comprises the sequence 5′-cauaguagc-3′, as shown in FIG. 2E. In some embodiments, the second polynucleotide comprises eight nucleotides and comprises the following features (5′ to 3′): a second side of the stem comprising eight nucleotides. In some embodiments, the second polynucleotide comprises the sequence 5′-gngaanuu-3′. In other embodiments, the second polynucleotide comprises the sequence 5′-gugaacug-3′, as shown in FIG. 2E.

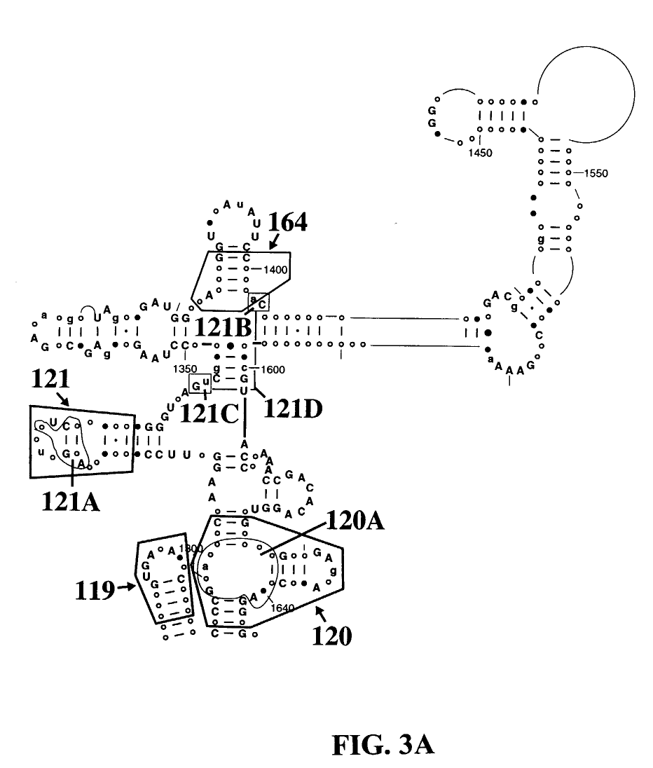

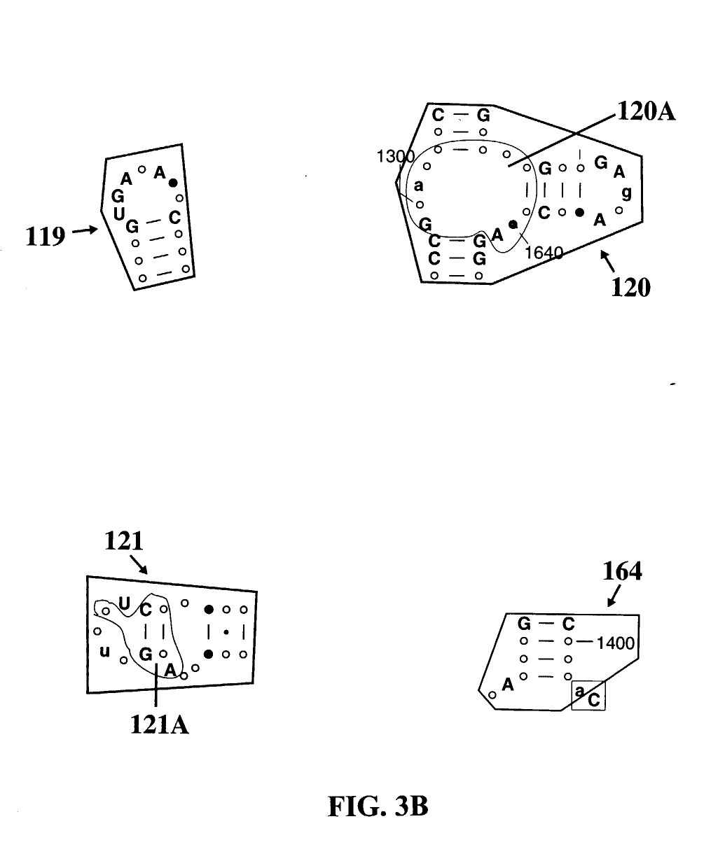

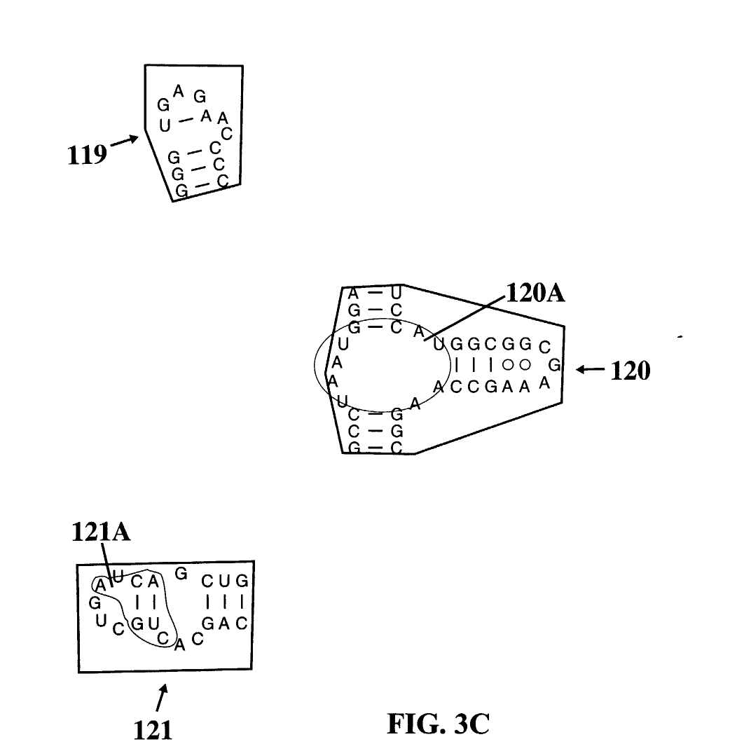

Consensus site 19, shown in FIG. 3A as region 119, comprises a region of RNA comprising from about eight nucleotides to about twenty two nucleotides, wherein portions of the polynucleotide form a double-stranded RNA having the following features (5′ to 3′): a first side of a stem comprising from about two nucleotides to about six nucleotides, a terminal loop comprising from about four nucleotides to about ten nucleotides, and a second side of the stem comprising from about two nucleotides to about six nucleotides.

In some embodiments, consensus site 19 comprises fifteen nucleotides, wherein portions thereof form a double-stranded RNA having the following features (5′ to 3′): a first side of a stem comprising four nucleotides, a terminal loop comprising seven nucleotides, and a second side of the stem comprising four nucleotides. In some embodiments, the polynucleotide comprises the sequence 5′-nnngugananncnnn-3′ (SEQ ID NO:138). In other embodiments, the polynucleotide comprises the sequence 5′-gggugagaacccc-3′ (SEQ ID NO:187), as shown in FIG. 3C.

Consensus site 20, shown in FIG. 3A as region 120, comprises a region of RNA comprising a first and second polynucleotide. The first polynucleotide comprises from about six nucleotides to about sixteen nucleotides and comprises the following features (5′ to 3′): a first side of a stem comprising from about two nucleotides to about five nucleotides, a bulge comprising from about two nucleotides to about six nucleotides, and a first side of a second stem comprising from about two nucleotides to about five nucleotides. The second polynucleotide comprises from about thirteen nucleotides to about thirty four nucleotides and comprises the following features (5′ to 3′): a second side of the second stem comprising from about two nucleotides to about five nucleotides, a bulge comprising from about one nucleotide to about two nucleotides, a first side of a third stem comprising from about two nucleotides to about six nucleotides, a terminal loop comprising from about three nucleotides to about seven nucleotides, a second side of the third stem comprising from about two nucleotides to about six nucleotides, a bulge comprising from about one nucleotide to about three nucleotides, and a second side of the first stem comprising from about two nucleotides to about five nucleotides. As shown in FIG. 3B, nucleotides within region 120A form a pocket.

In some embodiments, the first polynucleotide of consensus site 20 comprises ten nucleotides and comprises the following features (5′ to 3′): a first side of a stem comprising three nucleotides, a bulge comprising four nucleotides, and a first side of a second stem comprising three nucleotides. In some embodiments, the first polynucleotide comprises the sequence 5′-nccgnannnc-3′ (SEQ ID NO:139). In other embodiments, the polynucleotide comprises the sequence 5′-gccuaaugga-3′ (SEQ ID NO:188), as shown in FIG. 3C. In some embodiments, the second polynucleotide comprises twenty two nucleotides and comprises the following features (5′ to 3′): a second side of the second stem comprising three nucleotides, a bulge comprising one nucleotide, a first side of a third stem comprising four nucleotides, a terminal loop comprising five nucleotides, a second side of the third stem comprising four nucleotides, a bulge comprising two nucleotides, and a second side of the first stem comprising three nucleotides. In some embodiments, the second polynucleotide comprises the sequence 5′-gnnnngnngagnanncnnaggn-3′ (SEQ ID NO:140). In other embodiments, the polynucleotide comprises the sequence 5′-uccauggcggcgaaagccaaggc-3′ (SEQ ID NO:189), as shown in FIG. 3C.

Consensus site 21, shown in FIG. 3A as region 121, comprises a region of RNA comprising fromlabout eleven nucleotides to about twenty nine nucleotides, wherein portions of the polynucleotide form a double-stranded RNA having the following features (5′ to 3′): a first side of a first stem comprising from about two nucleotides to about five nucleotides, a first side of an internal loop comprising from about two nucleotides to about five nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about one nucleotide to about two nucleotides, and a second side of the first stem comprising from about two nucleotides to about five nucleotides. As shown in FIG. 3B, nucleotides within region 121A form a pocket.

In some embodiments, consensus site 21 comprises nineteen nucleotides, wherein portions thereof form a double-stranded RNA having the following features (5′ to 3′): a first side of a first stem comprising three nucleotides, a first side of an internal loop comprising three nucleotides, a first side of a second stem comprising two nucleotides, a terminal loop comprising four nucleotides, a second side of the second stem comprising two nucleotides, a second side of the internal loop comprising one nucleotide, and a second side of the first stem comprising three nucleotides. In some embodiments, the polynucleotide comprises the sequence 5′-nnnnnangnunnucnnnnn-3′ (SEQ ID NO:141). In other embodiments, the polynucleotide comprises the sequence 5′-cagcacugcugaucagcug-3′ (SEQ ID NO:186), as shown in FIG. 3C.

Consensus site 22, shown in FIG. 2A as region 122, comprises a region of RNA comprising a first and second polynucleotide. The first polynucleotide comprises from about six nucleotides to about sixteen nucleotides and comprises the following features (5′ to 3′): a first side of a stem comprising from about two nucleotides to about five nucleotides, a first side of an internal loop comprising from about two nucleotides to about six nucleotides, and a first side of a second stem comprising from about two nucleotides to about five nucleotides. The second polynucleotide comprises from about five nucleotides to about twelve nucleotides and comprises the following features (5′ to 3′): a second side of the second stem comprising from about two nucleotides to about five nucleotides, a second side of the internal loop comprising from about one nucleotide to about two nucleotides, and a second side of the first stem comprising from about two nucleotides to about five nucleotides. As shown in FIG. 2C, nucleotides within region 122A form a pocket.

In some embodiments, the first polynucleotide of consensus site 22 comprises ten nucleotides and comprises the following features (5′ to 3′): a first side of a stem comprising three nucleotides, a first side of an internal loop comprising four nucleotides, and a first side of a second stem comprising three nucleotides. In some embodiments, the first polynucleotide comprises the sequence 5′-gnnnnaannn-3′ (SEQ ID NO:142). In other embodiments, the polynucleotide comprises the sequence 5′-gggagcaacc-3′ (SEQ ID NO:184), as shown in FIG. 2E. In some embodiments, the second polynucleotide comprises seven nucleotides and comprises the following features (5′ to 3′): a second side of the second stem comprising three nucleotides, a second side of the internal loop comprising one nucleotide, and a second side of the first stem comprising three nucleotides. In some embodiments, the second polynucleotide comprises the sequence 5′-nnnncnc-3′. In other embodiments, the polynucleotide comprises the sequence 5′-gggccc-3′, as shown in FIG. 2E.

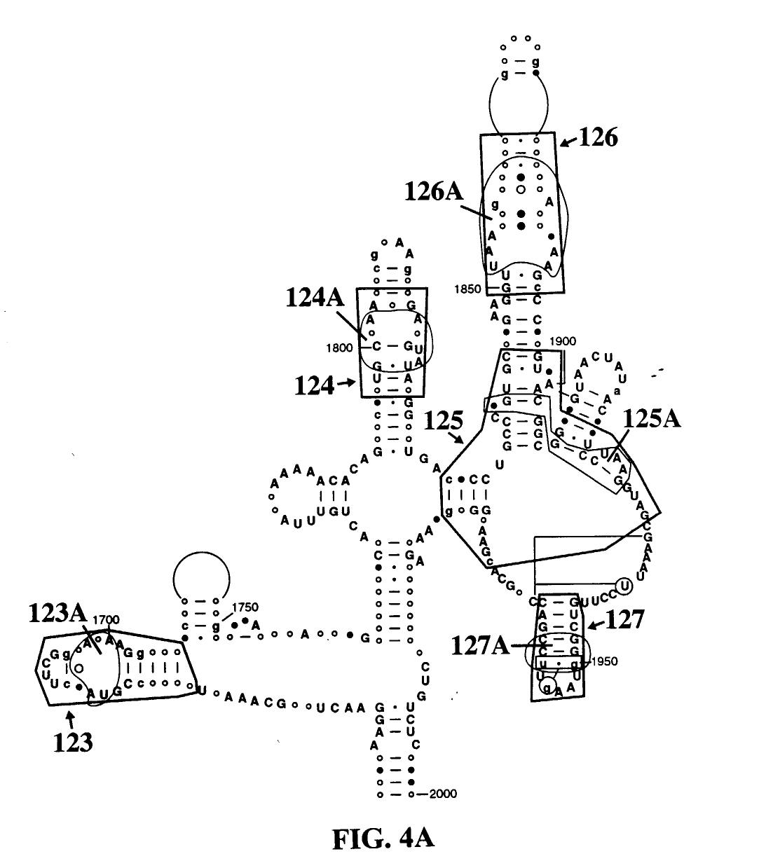

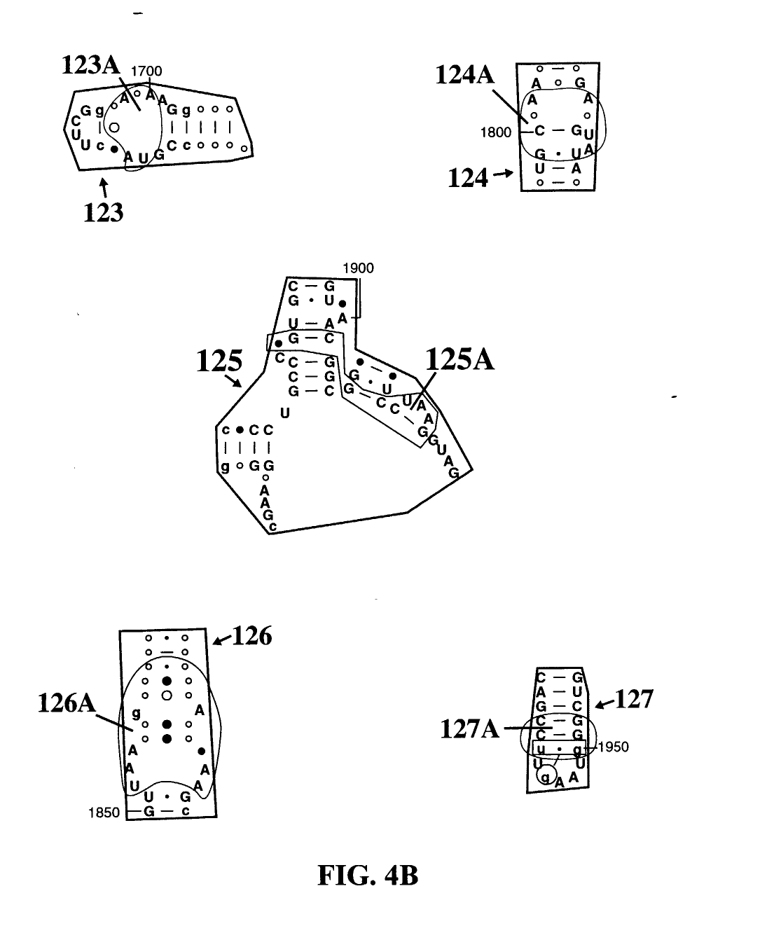

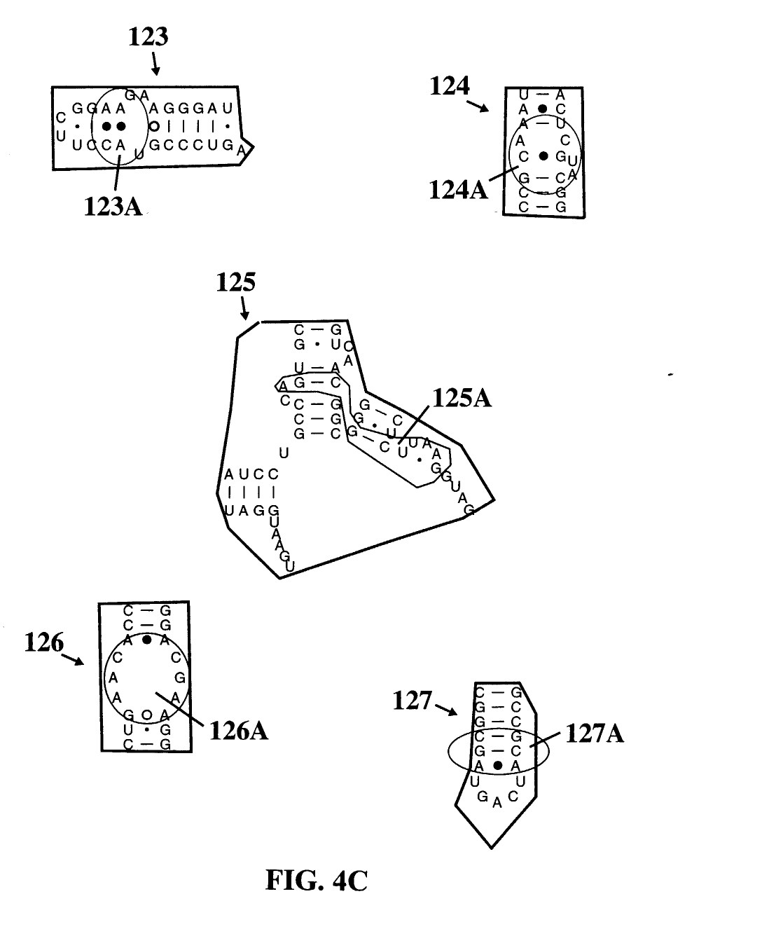

Consensus site 23, shown in FIG. 4A as region 123, comprises a region of RNA comprising from about fifteen nucleotides to about thirty nine nucleotides, wherein portions thereof form a double-stranded RNA having the following features (5′ to 3′): a dangling region comprising from about one nucleotide to about two nucleotides, a first side of a first stem comprising from about three nucleotides to about seven nucleotides, a first side of an internal loop comprising from about two nucleotides to about five nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about two nucleotides to about six nucleotides, and a second side of the first stem comprising from about three nucleotides to about seven nucleotides. As shown in FIG. 4B, nucleotides within region 123A form a pocket.

In some embodiments, consensus site 23 comprises twenty six nucleotides, wherein portions thereof form a double-stranded RNA having the following features (5′ to 3′): a dangling region comprising one nucleotide, a first side of a first stem comprising five nucleotides, a first side of an internal loop comprising three nucleotides, a first side of a second stem comprising two nucleotides, a terminal loop comprising four nucleotides, a second side of the second stem comprising two nucleotides, a second side of the internal loop comprising four nucleotides, and a second side of the first stem comprising five nucleotides. In some embodiments, the polynucleotide comprises the sequence 5′-nnnnccguancuucggnanaaggnnn-3′ (SEQ ID NO:143). In other embodiments, the polynucleotide comprises the sequence 5′-agucccguaccuucggaagaagggau-3′ (SEQ ID NO:190), as shown in FIG. 4C.

Consensus site 24, shown in FIG. 4A as region 124, comprises a region of RNA comprising a first and second polynucleotide. The first polynucleotide comprises from about four nucleotides to about twelve nucleotides and comprises the following features (5′ to 3′): a first side of a stem comprising from about three nucleotides to about nine nucleotides wherein a first side of an internal loop comprising from about one nucleotide to about three nucleotides is present in the first side of the stem. The second polynucleotide comprises from about five nucleotides to about fifteen nucleotides and comprises the following features (5′ to 3′): a second side of the stem comprising from about three nucleotides to about nine nucleotides wherein a second side of the internal loop comprising from about one nucleotide to about three nucleotides is present in the second side of the stem and wherein a bulge comprising from about one nucleotide to about three nucleotides is present in the second side of the stem. As shown in FIG. 4B, nucleotides within region 124A form a pocket.

In some embodiments, the first polynucleotide of consensus site 24 comprises eight nucleotides and comprises the following features (5′ to 3′): a first side of a stem comprising six nucleotides wherein a first side of an internal loop comprising two nucleotides is present between the fourth and fifth nucleotides of the first side of the stem. In some embodiments, the first polynucleotide comprises the sequence 5′-nugcnaan-3′. In other embodiments, the first polynucleotide comprises the sequence 5′-ccgcaaau-3′, as shown in FIG. 4C. In some embodiments, the second polynucleotide comprises ten nucleotides and comprises the following features (5′ to 3′): a second side of the stem comprising six nucleotides wherein a second side of the internal loop comprising two nucleotides is present between the second and third nucleotides of the second side of the stem and wherein a bulge comprising two nucleotides is present between the third and fourth nucleotides of the second side of the stem. In some embodiments, the second polynucleotide comprises the sequence 5′-nganguauan-3′ (SEQ ID NO:144). In other embodiments, the second polynucleotide comprises the sequence 5′-acucguacgg-3′ (SEQ ID NO:191), as shown in FIG. 4C.

Consensus site 25, shown in FIG. 4A as region 125, comprises a region of RNA comprising a first, second, third and fourth polynucleotide. The first polynucleotide comprises from about eight nucleotides to about twenty one nucleotides and comprises the following features (5′ to 3′): a first side of a first stem comprising from about two nucleotides to about six nucleotides, a bulge comprising from about one nucleotide to about two nucleotides, a first side of a second stem comprising from about four nucleotides to about ten nucleotides wherein a bulge comprising from about one nucleotide to about three nucleotides is present in the first side of the second stem. The second polynucleotide comprises from about seven nucleotides to about eighteen nucleotides and comprises the following features (5′ to 3′): a second side of the second stem comprising from about four nucleotides to about ten nucleotides wherein a bulge comprising from about one nucleotide to about three nucleotides is present in the second side of the second stem, and a first side of a third stem comprising from about two nucleotides to about five nucleotides. The third polynucleotide comprises from about eight nucleotides to about twenty nucleotides and comprises the following features (5′ to 3′): a second side of the third stem comprising from about two nucleotides to about five nucleotides, a first side of a fourth stem comprising from about one nucleotide to about two nucleotides, a terminal loop comprising from about two nucleotides to about five nucleotides, a second side of the fourth stem comprising from about one nucleotide to about two nucleotides, and a dangling region comprising from about two nucleotides to about six nucleotides. The fourth polynucleotide comprises from about five nucleotides to about thirteen nucleotides and comprises the following features (5′ to 3′): a dangling region comprising from about three nucleotides to about seven nucleotides, and a second side of the first stem comprising from about two nucleotides to about six nucleotides. As shown in FIG. 4B, nucleotides within region 125A form a pocket.

In some embodiments, the first polynucleotide of consensus site 25 comprises fourteen nucleotides and comprises the following features (5′ to 3′): a first side of a first stem comprising four nucleotides, a bulge comprising one nucleotide, a first side of a second stem comprising seven nucleotides wherein a bulge comprising two nucleotides is present between the third and fourth nucleotides of the first side of the second stem. In some embodiments, the first polynucleotide comprises the sequence 5′-cnccugcccngugc-3′ (SEQ ID NO:145). In other embodiments, the first polynucleotide comprises the sequence 5′-auccugcccagugc-3′ (SEQ ID NO:192), as shown in FIG. 4C. In some embodiments, the second polynucleotide comprises twelve nucleotides and comprises the following features (5′ to 3′): a second side of the second stem comprising seven nucleotides wherein a bulge comprising two nucleotides is present between the second and third nucleotides of the second side of the second stem, and a first side of a third stem comprising three nucleotides. In some embodiments, the second polynucleotide comprises the sequence 5′-gunaacggcggn-3′ (SEQ ID NO:146). In other embodiments, the second polynucleotide comprises the sequence 5′-gucaacggcggg-3′ (SEQ ID NO:193), as shown in FIG. 4C. In some embodiments, the third polynucleotide comprises twelve nucleotides and comprises the following features (5′ to 3′): a second side of the third stem comprising three nucleotides, a first side of a fourth stem comprising one nucleotide, a terminal loop comprising three nucleotides, a second side of the fourth stem comprising one nucleotide, and a dangling region comprising four nucleotides. In some embodiments, the third polynucleotide comprises the sequence 5′-nucuuaagguag-3′ (SEQ ID NO:147). In other embodiments, the third polynucleotide comprises the sequence 5′-cucuuaagguag-3′ (SEQ ID NO:194), as shown in FIG. 4C. In some embodiments, the fourth polynucleotide comprises nine nucleotides and comprises the following features (5′ to 3′): a dangling region comprising five nucleotides, and a second side of the first stem comprising four nucleotides. In some embodiments, the fourth polynucleotide comprises the sequence 5′-cgaanggng-3′ (SEQ ID NO:148). In other embodiments, the fourth polynucleotide comprises the sequence 5′-ugaauggau-3′, as shown in FIG. 4C. The molecular interaction site comprises a drug-binding pocket encompassing an area defined by about 13Å by 17Å and is located in the minor groove side of stems 68 and 69 and is centered around the 5 nucleotides immediately 3′ to stem 69.

Consensus site 26, shown in FIG. 4A as region 126, comprises a region of RNA comprising a first and second polynucleotide. The first polynucleotide comprises from about eight nucleotides to about twenty nucleotides and comprises the following features (5′ to 3′): a first side of a stem comprising from about five nucleotides to about thirteen nucleotides wherein a first side of a first internal loop comprising from about two nucleotides to about five nucleotides is present in the first side of the stem and wherein a first side of a second internal loop comprising from about one nucleotide to about two nucleotides is present in the first side of the stem. The second polynucleotide comprises from about eight nucleotides to about twenty nucleotides and comprises the following features (5′ to 3′): a second side of the stem comprising from about five nucleotides to about thirteen nucleotides wherein a second side of the second internal loop comprising from about one nucleotide to about two nucleotides is present in the second side of the stem and wherein a second side of the first internal loop comprising from about two nucleotides to about five nucleotides is present in the second side of the stem. As shown in FIG. 4B, nucleotides within region 126A form a pocket.

In some embodiments, the first polynucleotide of consensus site 26 comprises thirteen nucleotides and comprises the following features (5′ to 3′): a first side of a stem comprising nine nucleotides wherein a first side of a first internal loop comprising three nucleotides is present between the second and third nucleotides of the first side of the stem and wherein a first side of a second internal loop comprising one nucleotide is present between the fourth and fifth nucleotides of the first side of the stem. In some embodiments, the first polynucleotide comprises the sequence 5′-guuaanngnnnnn-3′ (SEQ ID NO:149). In other embodiments, the first polynucleotide comprises the sequence 5′-cugaacacc-3′, as shown in FIG. 4C. In some embodiments, the second polynucleotide comprises thirteen nucleotides and comprises the following features (5′ to 3′): a second side of the stem comprising nine nucleotides wherein a second side of the second internal loop comprising one nucleotide is present between the fifth and sixth nucleotides of the second side of the stem and wherein a second side of the first internal loop comprising three nucleotides is present between the seventh and eighth nucleotides of the second side of the stem. In some embodiments, the second polynucleotide comprises the sequence 5′-nnnnnannnaagc-3′ (SEQ ID NO:150). In other embodiments, the second polynucleotide comprises the sequence 5′-ggacgaagg-3′ (SEQ ID NO:326), as shown in FIG. 4C.

Consensus site 27, shown in FIG. 4A as region 127, comprises a region of RNA comprising from about nine nucleotides to about twenty five nucleotides, wherein portions thereof form a double-stranded RNA having the following features (5′ to 3′): a first side of a stem comprising from about three nucleotides to about nine nucleotides, a terminal loop comprising from about three nucleotides to about seven nucleotides, and a second side of the stem comprising from about three nucleotides to about nine nucleotides. As shown in FIG. 4B, nucleotides within region 127A form a pocket.

In some embodiments, consensus site 27 comprises seventeen nucleotides, wherein portions thereof form a double-stranded RNA having the following features (5′ to 3′): a first side of a stem comprising six nucleotides, a terminal loop comprising five nucleotides, and a second side of the stem comprising six nucleotides. In some embodiments, the polynucleotide comprises the sequence 5′-gucggguaaguuccgac-3′ (SEQ ID NO:151). In other embodiments, the polynucleotide comprises the sequence 5′-gccgcaucaguagcggc-3′ (SEQ ID NO:195), as shown in FIG. 4C. The molecular interaction site comprises a drug-binding pocket encompassing an area defined by about 17 Å by 13Å and encompasses the major groove of the entire loop 71, as well as the last two base-pairs of the closing stem.

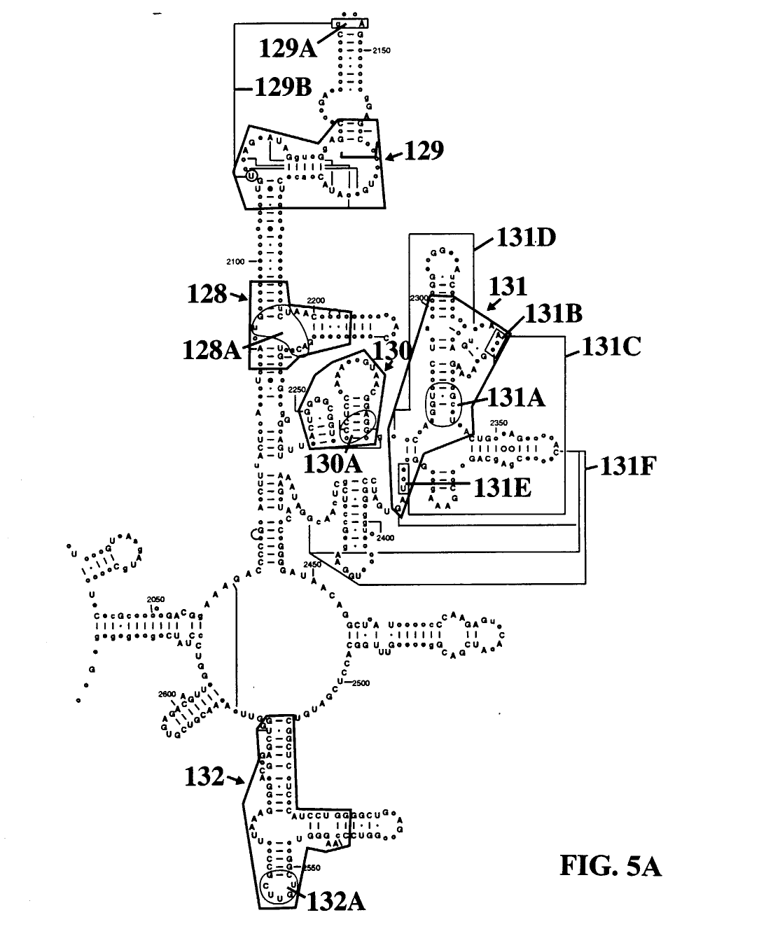

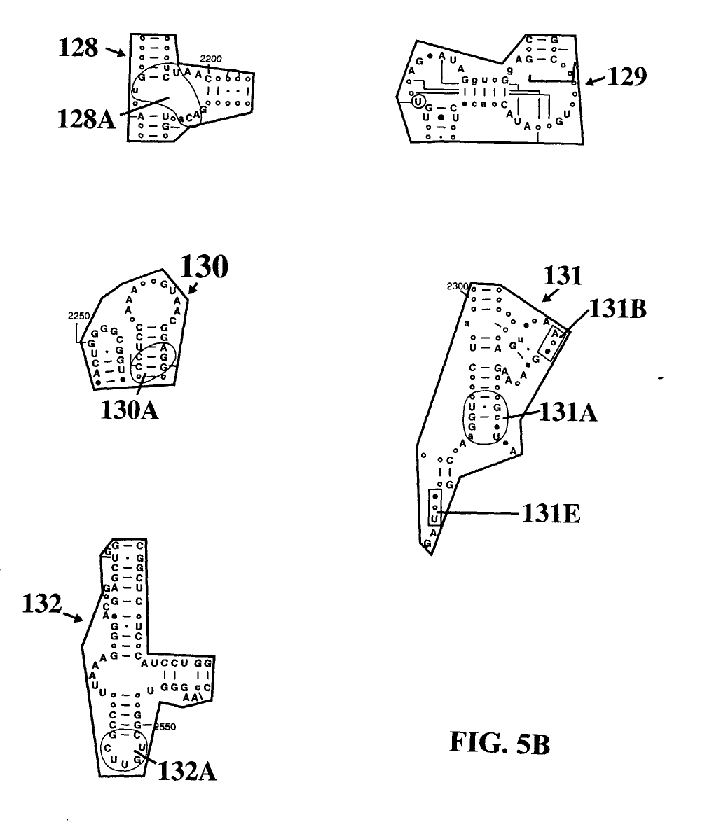

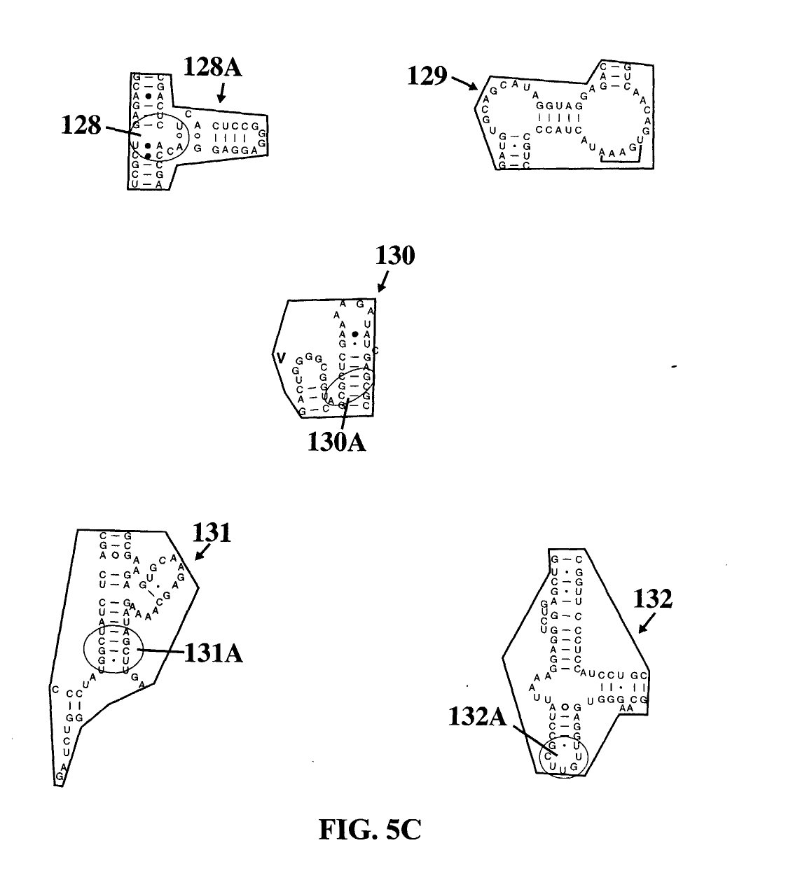

Consensus site 28, shown in FIG. 5A as region 128, comprises a region of RNA comprising a first, second and third polynucleotide. The first polynucleotide comprises from about six nucleotides to about fifteen nucleotides and comprises the following features (5′ to 3′): a first side of a first stem comprising from about two nucleotides to about five nucleotides, a bulge comprising from about one nucleotide to about three nucleotides, and a first side of a second stem comprising from about three nucleotides to about seven nucleotides. The second polynucleotide comprises from about nine nucleotides to about twenty one nucleotides and comprises the following features (5′ to 3′): a second side of the second stem comprising from about three nucleotides to about seven nucleotides, a first side of an internal loop comprising from about two nucleotides to about five nucleotides, and a first side of a third stem comprising from about three nucleotides to about seven nucleotides wherein a bulge comprising from about one nucleotide to about two nucleotides is optionally present in the first side of the third stem. The third polynucleotide comprises from about eight nucleotides to about nineteen nucleotides and comprises the following features (5′ to 3′): a second side of the third stem comprising from about three nucleotides to about seven nucleotides, a second side of the internal loop comprising from about three nucleotides to about seven nucleotides, and a second side of the first stem comprising from about two nucleotides to about five nucleotides. As shown in FIG. 5B, nucleotides within region 128A form a pocket.

In some embodiments, the first polynucleotide of consensus site 28 comprises ten nucleotides and comprises the following features (5′ to 3′): a first side of a first stem comprising three nucleotides, a bulge comprising two nucleotides, and a first side of a second stem comprising five nucleotides. In some embodiments, the first polynucleotide comprises the sequence 5′-nnanugnnnn-3′ (SEQ ID NO:152). In other embodiments, the first polynucleotide comprises the sequence 5′-ucgcugagacg-3′ (SEQ ID NO:196), as shown in FIG. 5C. In some embodiments, the second polynucleotide comprises thirteen or fourteen nucleotides and comprises the following features (5′ to 3′): a second side of the second stem comprising five nucleotides, a first side of an internal loop comprising three nucleotides, and a first side of a third stem comprising five nucleotides wherein a bulge comprising one nucleotide is optionally present between the third and fourth nucleotides of the first side of the third stem. In some embodiments, the second polynucleotide comprises the sequence 5′-nnnucuaacnnnn-3′ (SEQ ID NO:153) or 5′-nnnucuaacnnnnn-3′ (SEQ ID NO:154). In some embodiments, the third polynucleotide comprises thirteen nucleotides and comprises the following features (5′ to 3′): a second side of the third stem comprising five nucleotides, a second side of the internal loop comprising five nucleotides, and a second side of the first stem comprising three nucleotides. In some embodiments, the third polynucleotide comprises the sequence 5′-nnnnngacanugn-3′ (SEQ ID NO:155). In other embodiments, the second and third polynucleotides, together, comprises the sequence 5′-cgacucucacuccgggaggaggacaccga-3′ (SEQ ID NO:197), as shown in FIG. 5C.

Consensus site 29, shown in FIG. 5A as region 129, comprises a region of RNA comprising a first and second polynucleotide. The first polynucleotide comprises from about thirteen nucleotides to about thirty four nucleotides and comprises the following features (5′ to 3′): a first side of a first stem comprising from about two nucleotides to about six nucleotides, a bulge comprising from about five nucleotides to about thirteen nucleotides, a first side of a second stem comprising from about three nucleotides to about seven nucleotides, a first side of an internal loop comprising from about one nucleotide to about three nucleotides, and a first side of a third stem comprising from about two nucleotides to about five nucleotides. The second polynucleotide comprises from about thirteen nucleotides to about thirty six nucleotides and comprises the following features (5′ to 3′): a second side of the third stem comprising from about two nucleotides to about five nucleotides, a second side of an internal loop comprising from about six nucleotides to about eighteen nucleotides, a second side of the second stem comprising from about three nucleotides to about seven nucleotides, and a second side of the first stem comprising from about two nucleotides to about six nucleotides.

In some embodiments, the first polynucleotide of consensus site 29 comprises twenty three nucleotides and comprises the following features (5′ to 3′): a first side of a first stem comprising four nucleotides, a bulge comprising nine nucleotides, a first side of a second stem comprising five nucleotides, a first side of an internal loop comprising two nucleotides, and a first side of a third stem comprising three nucleotides. In some embodiments, the first polynucleotide comprises the sequence 5′-nnugunnagnauaggunggagnc-3′ (SEQ ID NO:156). In other embodiments, the first polynucleotide comprises the sequence 5′-gaugugcagcauagguaggagac-3′ (SEQ ID NO:198), as shown in FIG. 5C. In some embodiments, the second polynucleotide comprises twenty four nucleotides and comprises the following features (5′ to 3′): a second side of the third stem comprising three nucleotides, a second side of an internal loop comprising twelve nucleotides, a second side of the second stem comprising five nucleotides, and a second side of the first stem comprising four nucleotides. In some embodiments, the second polynucleotide comprises the sequence 5′-gncnnnnnugnnauacnacncunn-3′ (SEQ ID NO:157). In other embodiments, the second polynucleotide comprises the sequence 5′-gucaacagugaaauacuacccguc-3′ (SEQ ID NO:199), as shown in FIG. 5C.

Consensus site 30, shown in FIG. 5A as region 130, comprises a region of RNA comprising from about nineteen nucleotides to about fifty three nucleotides, wherein portions thereof form a double-stranded RNA having the following features (5′ to 3′): a first side of a first stem comprising from about two nucleotides to about six nucleotides, a terminal loop comprising from about three nucleotides to about seven nucleotides, a second side of the first stem comprising from about two nucleotides to about six nucleotides, a first side of a second stem comprising from about three nucleotides to about nine nucleotides, a terminal loop comprising from about six nucleotides to about sixteen nucleotides, and a second side of the second stem comprising from about three nucleotides to about nine nucleotides. As shown in FIG. 5B, nucleotides within region 130A form a pocket.

In some embodiments, consensus site 30 comprises thirty six nucleotides, wherein portions thereof form a double-stranded RNA having the following features (5′ to 3′): a first side of a first stem comprising four nucleotides, a terminal loop comprising five nucleotides, a second side of the first stem comprising four nucleotides, a first side of a second stem comprising six nucleotides, a terminal loop comprising eleven nucleotides, and a second side of the second stem comprising six nucleotides. In some embodiments, the polynucleotide comprises the sequence 5′-nacuggggcggunnccuccnaaannguaacggaggn-3′ (SEQ ID NO:158). In other embodiments, the polynucleotide comprises the sequence 5′-gacuggggcgguacgcgcucgaaaagauaucgagcgc-3′ (SEQ ID NO:200), as shown in FIG. 5C.

Consensus site 31, shown in FIG. 5A as region 131, comprises a region of RNA comprising a first, second and third polynucleotide. The first polynucleotide comprises from about eleven nucleotides to about twenty nine nucleotides and comprises the following features (5′ to 3′): a dangling region comprising from about one nucleotide to about two nucleotides, a first side of a first stem comprising from about one nucleotide to about three nucleotides, a bulge comprising from about two nucleotides to about six nucleotides, a first side of a second stem comprising from about three nucleotides to about nine nucleotides, and a first side of a third stem comprising from about three nucleotides to about seven nucleotides wherein a bulge comprising from about one nucleotide to about two nucleotides is present in the first side of the third stem. The second polynucleotide comprises from about seventeen nucleotides to about forty eight nucleotides and comprises the following features (5′ to 3′): a second side of the third stem comprising from about three nucleotides to about seven nucleotides wherein a bulge comprising from about two nucleotides to about five nucleotides is present in the second side of the third stem, a first side of a fourth stem comprising from about one nucleotide to about three nucleotides, a terminal loop comprising from about three nucleotides to about nine nucleotides, a second side of the fourth stem comprising from about one nucleotide to about three nucleotides, a bulge comprising from about two nucleotides to about six nucleotides, a second side of the second stem comprising from about three nucleotides to about nine nucleotides, and a dangling region comprising from about two nucleotides to about six nucleotides. The third polynucleotide comprises from about four nucleotides to about ten nucleotides and comprises the following features (5′ to 3′): a second side of the first stem comprising from about one nucleotide to about three nucleotides and a dangling region comprising from about three nucleotides to about seven nucleotides. As shown in FIGS. 5A and 5B, nucleotides within region 131A form a pocket. Nucleotides within region 131B form an interaction (131C) with nucleotides within region 131E.

In some embodiments, the first polynucleotide of consensus site 31 comprises nineteen nucleotides and comprises the following features (5′ to 3′): a dangling region comprising one nucleotide, a first side of a first stem comprising two nucleotides, a bulge comprising four nucleotides, a first side of a second stem comprising six nucleotides, and a first side of a third stem comprising five nucleotides wherein a bulge comprising one nucleotide is present between the second and third nucleotides of the first side of the third stem. In some embodiments, the first polynucleotide comprises the sequence 5′-nncnaaggunnncunannn-3′ (SEQ ID NO:159). In other embodiments, the first polynucleotide comprises the sequence 5′-cccuauggcuaucucagc-3′ (SEQ ID NO:201), as shown in FIG. 5C. In some embodiments, the second polynucleotide comprises thirty two nucleotides and comprises the following features (5′ to 3′): a second side of the third stem comprising five nucleotides wherein a bulge comprising three nucleotides is present between the third and fourth nucleotides of the second side of the third stem, a first side of a fourth stem comprising two nucleotides, a terminal loop comprising six nucleotides, a second side of the fourth stem comprising two nucleotides, a bulge comprising four nucleotides, a second side of the second stem comprising six nucleotides, and a dangling region comprising four nucleotides. In some embodiments, the second polynucleotide comprises the sequence 5′-nnnnnnnagunnaanngnanaagnnngcnuna-3′ (SEQ ID NO:160). In other embodiments, the second polynucleotide comprises the sequence 5′-gcgaagagugcaagagcaaaagauagcuuga-3′ (SEQ ID NO:202), as shown in FIG. 5C. In some embodiments, the third polynucleotide comprises seven nucleotides and comprises the following features (5′ to 3′): a second side of the first stem comprising two nucleotides and a dangling region comprising five nucleotides. In some embodiments, the third polynucleotide comprises the sequence 5′-gnnnuag-3′. In other embodiments, the third polynucleotide comprises the sequence 5′-ggucuag-3′, as shown in FIG. 5C.

Consensus site 32, shown in FIG. 5A as region 132, comprises a region of RNA comprising a first and second polynucleotide. The first polynucleotide comprises from about ten nucleotides to about twenty seven nucleotides and comprises the following features (5′ to 3′): a first side of a first stem comprising from about six nucleotides to about sixteen nucleotides, a bulge comprising from about one nucleotide to about three nucleotides, and a first side of a second stem comprising from about two nucleotides to about six nucleotides wherein a first side of an internal loop comprising from about one nucleotide to about two nucleotides is present in the first side of the second stem. The second polynucleotide comprises from about twenty six nucleotides to about sixty five nucleotides and comprises the following features (5′ to 3′): a second side of the second stem comprising from about two nucleotides to about six nucleotides a second side of the internal loop comprising from about two nucleotides to about five nucleotides is present in the second side of the second stem, a bulge comprising from about one nucleotide to about two nucleotides, a first side of a third stem comprising from about three nucleotides to about seven nucleotides, a terminal loop comprising from about three nucleotides to about seven nucleotides, a second side of the third stem comprising from about three nucleotides to about seven nucleotides, a bulge comprising from about three nucleotides to about seven nucleotides, and a second side of the first stem comprising from about six nucleotides to about sixteen nucleotides wherein a bulge comprising from about one nucleotide to about three nucleotides is present in the second side of the first stem and wherein a bulge comprising from about one nucleotide to about three nucleotides is present in the second side of the first stem and wherein a bulge comprising from about one nucleotide to about two nucleotides is present in the second side of the first stem. As shown in FIG. 5B, nucleotides within region 132A form a pocket.

In some embodiments, the first polynucleotide of consensus site 32 comprises eighteen nucleotides and comprises the following features (5′ to 3′): a first side of a first stem comprising eleven nucleotides, a bulge comprising two nucleotides, and a first side of a second stem comprising four nucleotides wherein a first side of an internal loop comprising one nucleotide is present between the second and third nucleotides of the first side of the second stem. In some embodiments, the first polynucleotide comprises the sequence 5′-cggcucnucncauccugg-3′ (SEQ ID NO:161). In other embodiments, the first polynucleotide comprises the sequence 5′-cgguucccuccauccugc-3′ (SEQ ID NO:203), as shown in FIG. 5C. In some embodiments, the second polynucleotide comprises forty four nucleotides and comprises the following features (5′ to 3′): a second side of the second stem comprising four nucleotides a second side of the internal loop comprising three nucleotides is present between the second and third nucleotides of the second side of the second stem, a bulge comprising one nucleotide, a first side of a third stem comprising five nucleotides, a terminal loop comprising five nucleotides, a second side of the third stem comprising five nucleotides, a bulge comprising five nucleotides, and a second side of the first stem comprising eleven nucleotides wherein a bulge comprising two nucleotides is present between the fifth and sixth nucleotides of the second side of the first stem and wherein a bulge comprising two nucleotides is present between the sixth and seventh nucleotides of the second side of the first stem and wherein a bulge comprising one nucleotide is present between the tenth and eleventh nucleotides of the second side of the first stem. In some embodiments, the second polynucleotide comprises the sequence 5′-ccaagggunnggcuguucgccnnuuaaag nggnacgngagcugg-3′ (SEQ ID NO:162). In other embodiments, the second polynucleotide comprises the sequence 5′-gcaagggugagguuguucgccuauuaaaggaggucg ugagcug-3′ (SEQ ID NO:327), as shown in FIG. 5C.

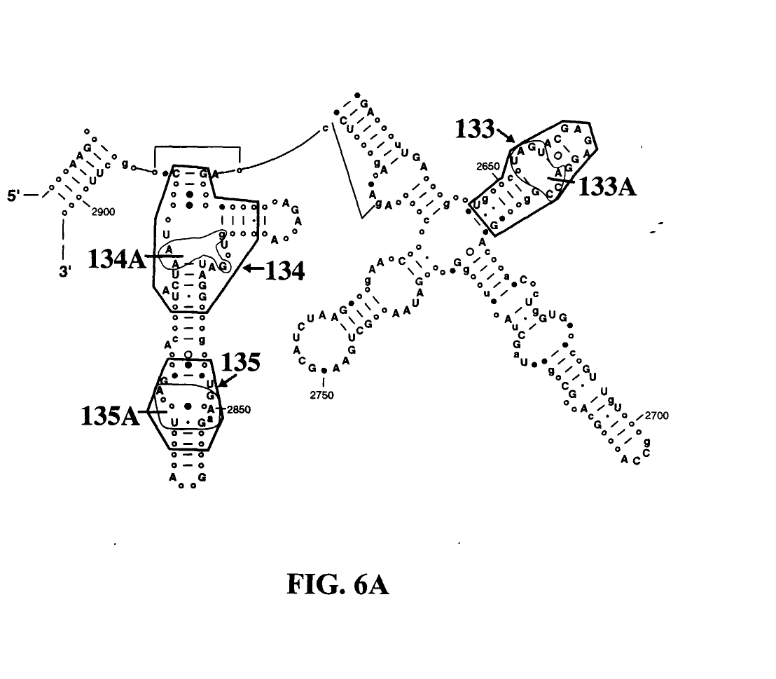

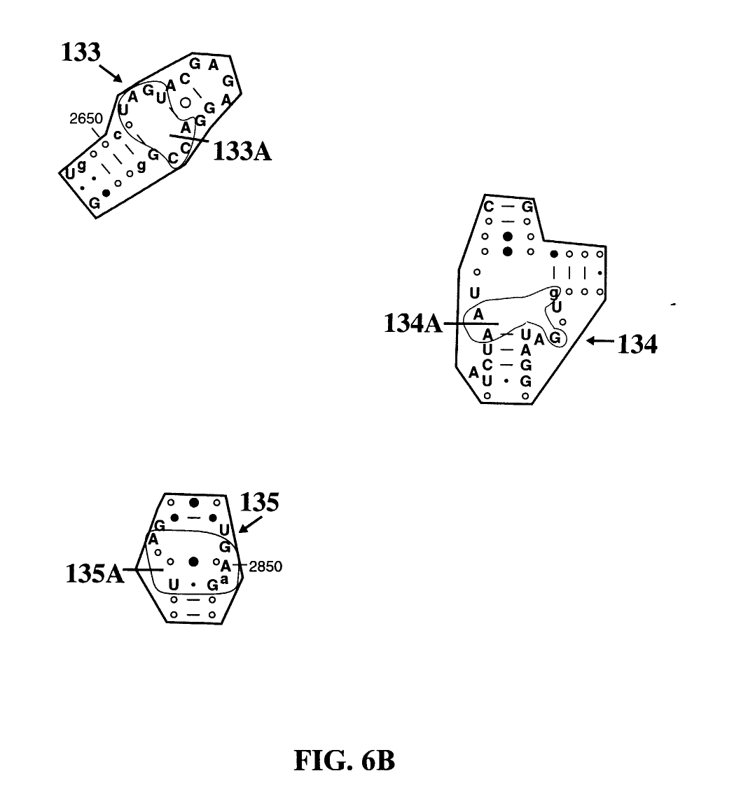

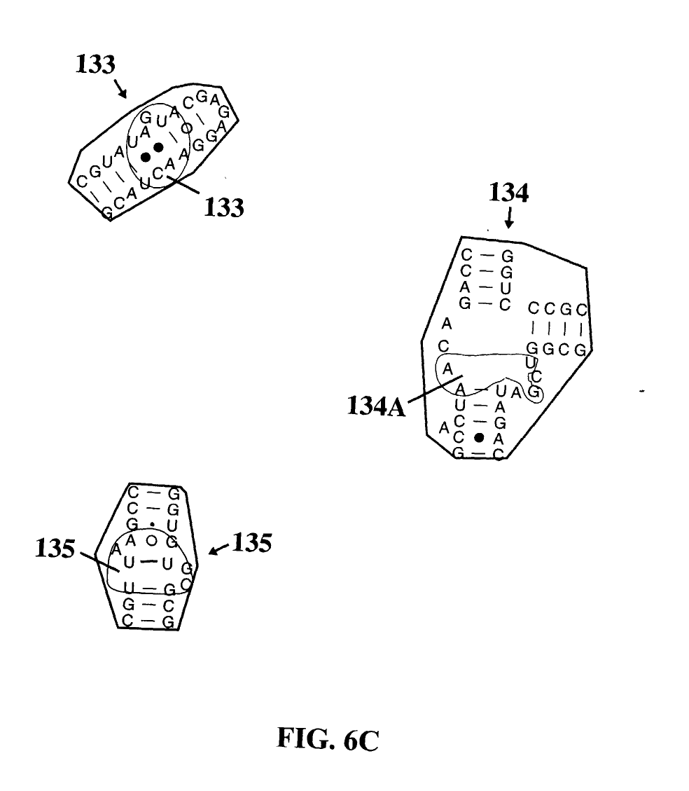

Consensus site 33, shown in FIG. 6A as region 133, comprises a region of RNA comprising from about fifteen nucleotides to about forty nucleotides, wherein portions of the polynucleotide form a double-stranded RNA having the following features (5′ to 3′): a first side of a stem comprising from about five nucleotides to about thirteen nucleotides wherein a first side of an internal loop comprising from about two nucleotides to about five nucleotides is present in the first side of the stem, a terminal loop comprising from about two nucleotides to about six nucleotides, and a second side of the first stem comprising from about five nucleotides to about thirteen nucleotides wherein a second side of the internal loop comprising from about one nucleotide to about three nucleotides is present in the second side of the stem. As shown in FIG. 6B, nucleotides within region 133A form a pocket.

In some embodiments, consensus site 33 comprises twenty seven nucleotides, wherein portions thereof form a double-stranded RNA having the following features (5′ to 3′): a first side of a stem comprising nine nucleotides wherein a first side of an internal loop comprising three nucleotides is present between the sixth and seventh nucleotides of the first side of the stem, a terminal loop comprising four nucleotides, and a second side of the first stem comprising nine nucleotides wherein a second side of the internal loop comprising two nucleotides is present between the third and fourth nucleotides of the second side of the stem. In some embodiments, the polynucleotide comprises the sequence 5′-ugnncnuaguacgagaggaccggnnng-3′ (SEQ ID NO:163). In other embodiments, the polynucleotide comprises the sequence 5′-cguauaguacgagaggaacuacg-3′ (SEQ ID NO:204), as shown in FIG. 6C. The molecular interaction site comprises a drug-binding pocket encompassing an area defined by about 15 Å by 10 Å, and lies into the major groove of stem 95, and is centered around the nucleotides U2653 and A2654.

Consensus site 34, shown in FIG. 6A as region 134, comprises a region of RNA comprising a first, second and third polynucleotide. The first polynucleotide comprises from about four nucleotides to about twelve nucleotides and comprises the following features (5′ to 3′): a first side of a first stem comprising from about two nucleotides to about six nucleotides and a first side of a second stem comprising from about two nucleotides to about six nucleotides. The second polynucleotide comprises from about seven nucleotides to about nineteen nucleotides and comprises the following features (5′ to 3′): a second side of the second stem comprising from about two nucleotides to about six nucleotides, a bulge comprising from about two nucleotides to about six nucleotides, and a first side of a third stem comprising from about three nucleotides to about seven nucleotides. The third polynucleotide comprises from about eight nucleotides to about twenty nucleotides and comprises the following features (5′ to 3′): a second side of the third stem comprising from about three nucleotides to about seven nucleotides wherein a bulge comprising from about one nucleotide to about two nucleotides is present in the second side of the third stem, a bulge comprising from about two nucleotides to about five nucleotides, and second side of the first stem comprising from about two nucleotides to about six nucleotides. As shown in FIG. 6B, nucleotides within region 134A form a pocket.

In some embodiments, the first polynucleotide of consensus site 34 comprises eight nucleotides and comprises the following features (5′ to 3′): a first side of a first stem comprising four nucleotides and a first side of a second stem comprising four eight nucleotides. In some embodiments, the first polynucleotide comprises the sequence 5′-gnnnnnnn-3′. In other embodiments, the first polynucleotide comprises the sequence 5′-ggucccgc-3′, as shown in FIG. 6C. In some embodiments, the second polynucleotide comprises thirteen nucleotides and comprises the following features (5′ to 3′): a second side of the second stem comprising four nucleotides, a bulge comprising four nucleotides, and a first side of a third stem comprising five nucleotides. In some embodiments, the second polynucleotide comprises the sequence 5′-nnngungauaggn-3′ (SEQ ID NO:164). In other embodiments, the second polynucleotide comprises the sequence 5′-gcggucgauagac-3′ (SEQ ID NO:205), as shown in FIG. 6C. In some embodiments, the third polynucleotide comprises thirteen nucleotides and comprises the following features (5′ to 3′): a second side of the third stem comprising five nucleotides wherein a bulge comprising one nucleotide is present between the second and third nucleotides of the second side of the third stem, a bulge comprising three nucleotides, and second side of the first stem comprising four nucleotides. In some embodiments, the third polynucleotide comprises the sequence 5′-nuacuaaunnnnc-3′ (SEQ ID NO:165). In other embodiments, the third polynucleotide comprises the sequence 5′-gcacuaacagacc-3′ (SEQ ID NO:206), as shown in FIG. 6C.

Consensus site 35, shown in FIG. 6A as region 135, comprises a region of RNA comprising a first and second polynucleotide. The first polynucleotide comprises from about five nucleotides to about fifteen nucleotides and comprises the following features (5′ to 3′): a first side of a stem comprising from about three nucleotides to about nine nucleotides wherein a first side of an internal loop comprising from about one nucleotide to about three nucleotides is present between the second and third nucleotides of the first side of the stem and wherein a bulge comprising from about one nucleotide to about three nucleotides is present between the third and fourth nucleotides of the first side of the stem. The second polynucleotide comprises from about five nucleotides to about fifteen nucleotides and comprises the following features (5′ to 3′): a second side of the stem comprising from about three nucleotides to about nine nucleotides wherein a second side of the internal loop comprising from about two nucleotides to about six nucleotides is present in the second side of the stem. As shown in FIG. 6B, nucleotides within region 135A form a pocket.

In some embodiments, the first polynucleotide of consensus site 35 comprises ten nucleotides and comprises the following features (5′ to 3′): a first side of a stem comprising six nucleotides wherein a first side of an internal loop comprising two nucleotides is present between the second and third nucleotides of the first side of the stem and wherein a bulge comprising two nucleotides is present between the third and fourth nucleotides of the first side of the stem. In some embodiments, the first polynucleotide comprises the sequence 5′-nnugnaagnn-3′ (SEQ ID NO:166). In other embodiments, the first polynucleotide comprises the sequence 5′-ggugugcgcg-3′ (SEQ ID NO:207), as shown in FIG. 6C. In some embodiments, the second polynucleotide comprises nine nucleotides and comprises the following features (5′ to 3′): a second side of the stem comprising six nucleotides wherein a second side of the internal loop comprising four nucleotides is present between the fourth and fifth nucleotides of the second side of the stem. In some embodiments, the second polynucleotide comprises the sequence 5′-nnunnagnn-3′. In other embodiments, the second polynucleotide comprises the sequence 5′-cguuaagcc-3′, as shown in FIG. 6C.

Consensus site 36, shown in FIG. 2A as region 165, comprises a region of RNA comprising from about eleven nucleotides to about thirty three nucleotides comprising the following features (5′ to 3′): a first side of a stem comprising from about four nucleotides to about twelve nucleotides, a terminal loop comprising from about three nucleotides to about nine nucleotides, and a second side of the stem comprising about four nucleotides to about twelve nucleotides.

In some embodiments, consensus site 36 comprises twenty two nucleotides and comprises the following features (5′ to 3′): a first side of a stem comprising eight nucleotides, a terminal loop comprising six nucleotides, and a second side of the stem comprising eight nucleotides. In some embodiments, the polynucleotide comprises the sequence 5′-cnnngngngnuaannuncnnng-3′ (SEQ ID NO:167). In other embodiments, the polynucleotide comprises the sequence 5′-ugcgcgggguaagccugugua-3′ (SEQ ID NO:185).

Consensus site 37, shown in FIG. 3A as region 164, comprises a region of RNA comprising a first and second polynucleotide. The first polynucleotide comprises from about three nucleotides to about nine nucleotides and comprises the following features (5′ to 3′): a dangling region comprising from about one to about three nucleotides, and a first side of a stem comprising from about two nucleotides to about six nucleotides. The second polynucleotide comprises from about three nucleotides to about nine nucleotides and comprises the following features (5′ to 3′): a second side of the stem comprising from about two nucleotides to about six nucleotides, and a dangling region comprising about one to about three nucleotides.

In some embodiments, the first polynucleotide of consensus site 37 comprises six nucleotides and comprises the following features (5′ to 3′): a dangling region comprising two nucleotides, and a first side of a stem comprising four nucleotides. In some embodiments, the first polynucleotide comprises the sequence 5′-nannng-3′. In other embodiments, the first polynucleotide comprises the sequence 5′-aaaccg-3′, as shown in FIG. 3C. In some embodiments, the second polynucleotide comprises six nucleotides and comprises the following features (5′ to 3′): a second side of the stem comprising four nucleotides, and a dangling region comprising two nucleotides. In some embodiments, the second polynucleotide comprises the sequence 5′-cnnnac-3′. In other embodiments, the second polynucleotide comprises the sequence 5′-ccgugc-3′, as shown in FIG. 3C.

### Example 3

### Molecular Interaction Sites In Additional 23S rRNA Species

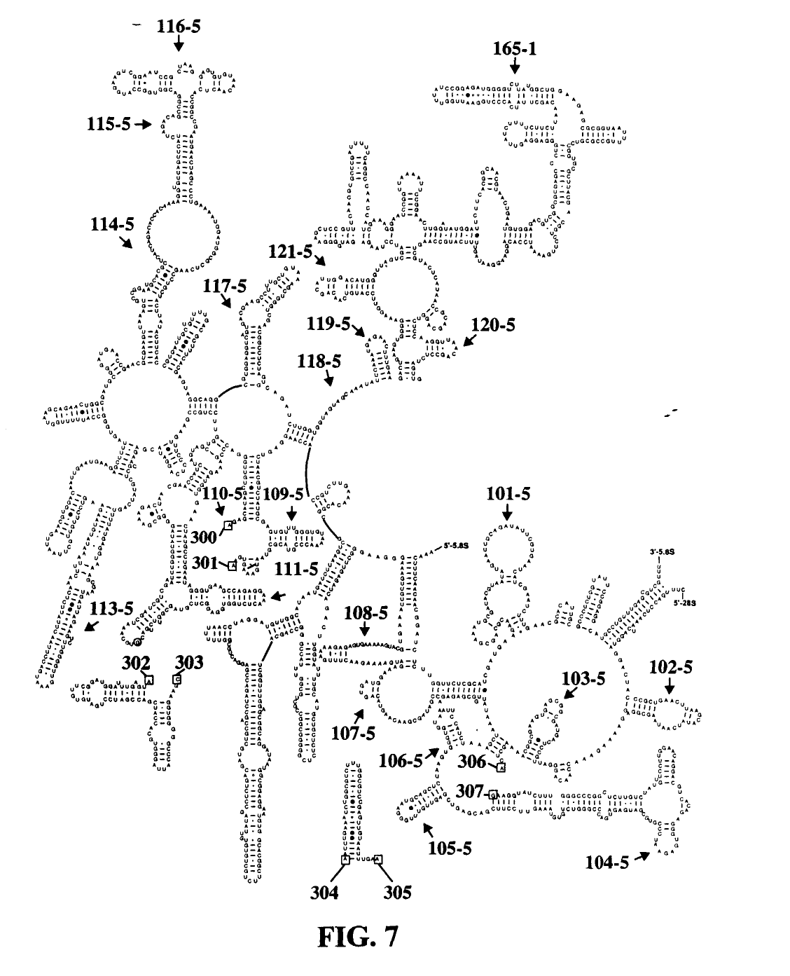

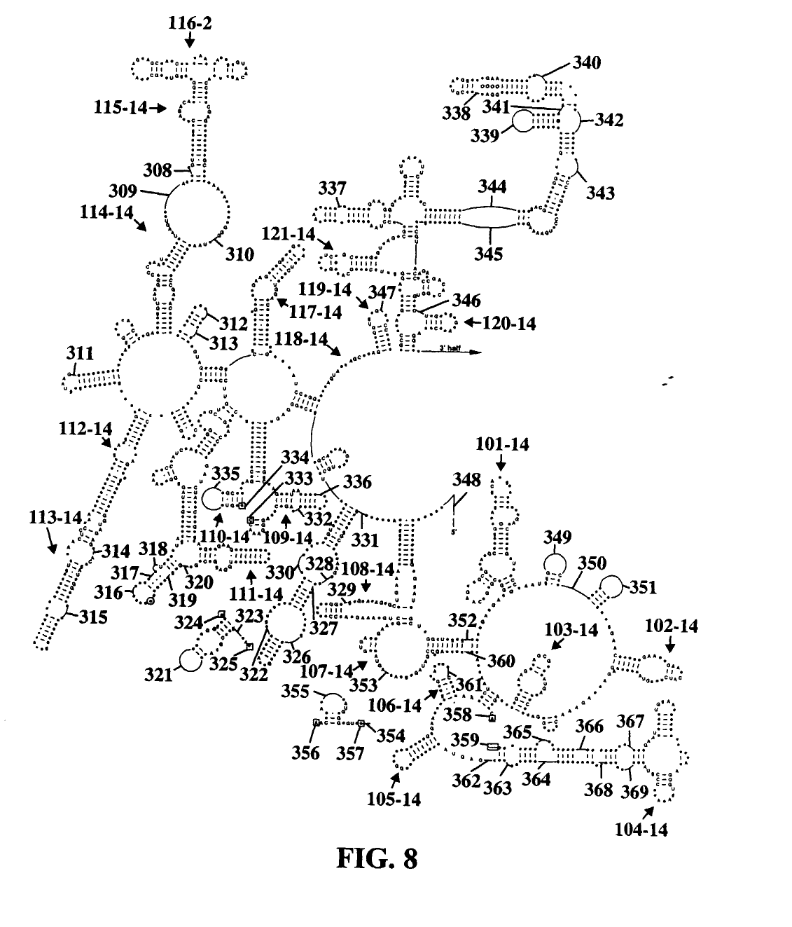

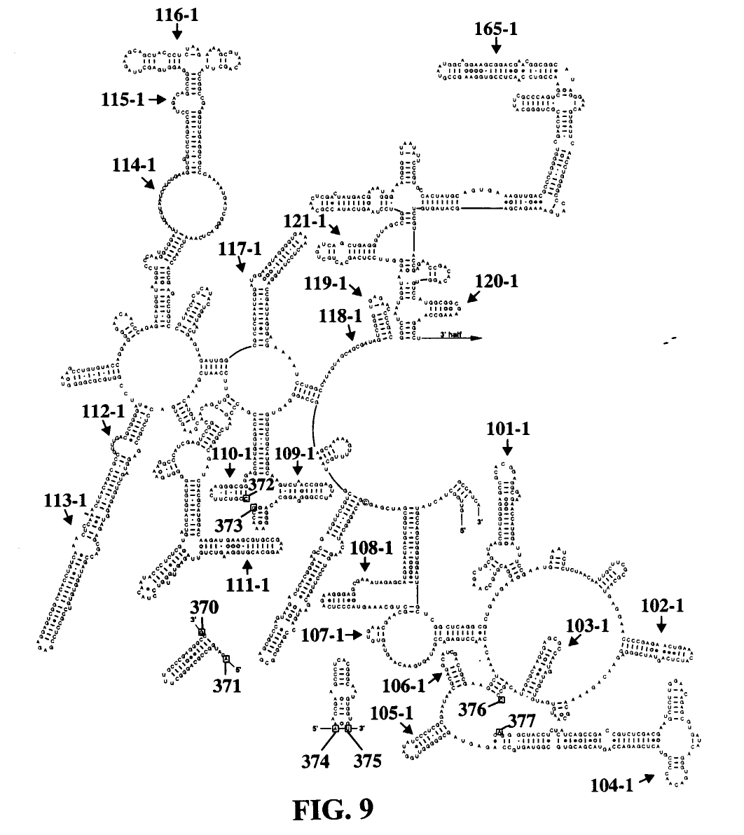

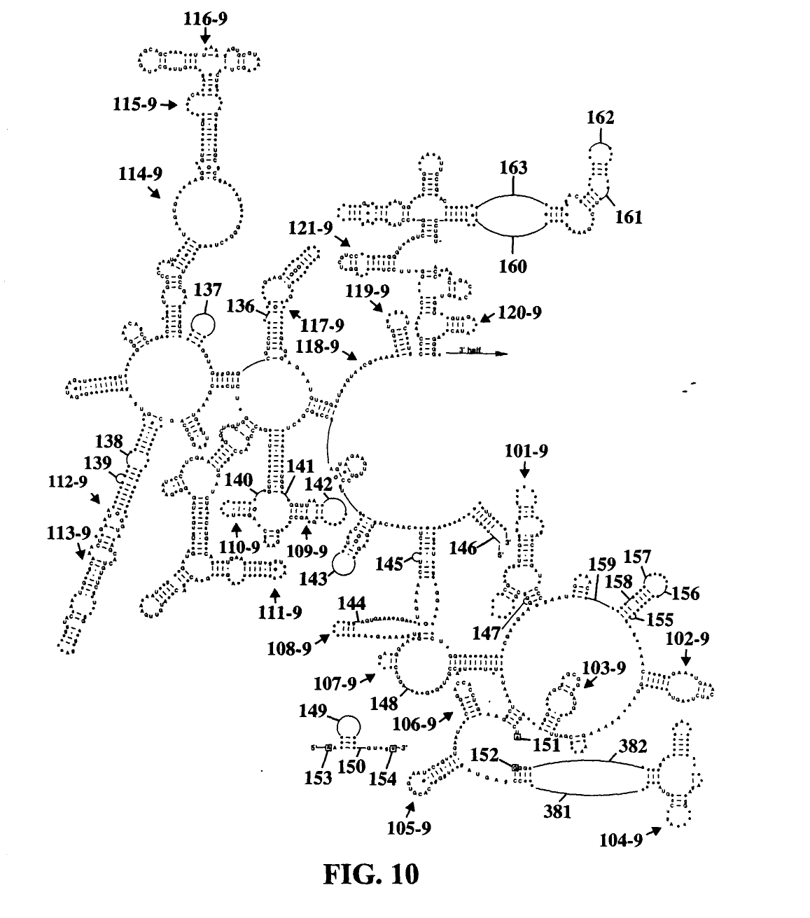

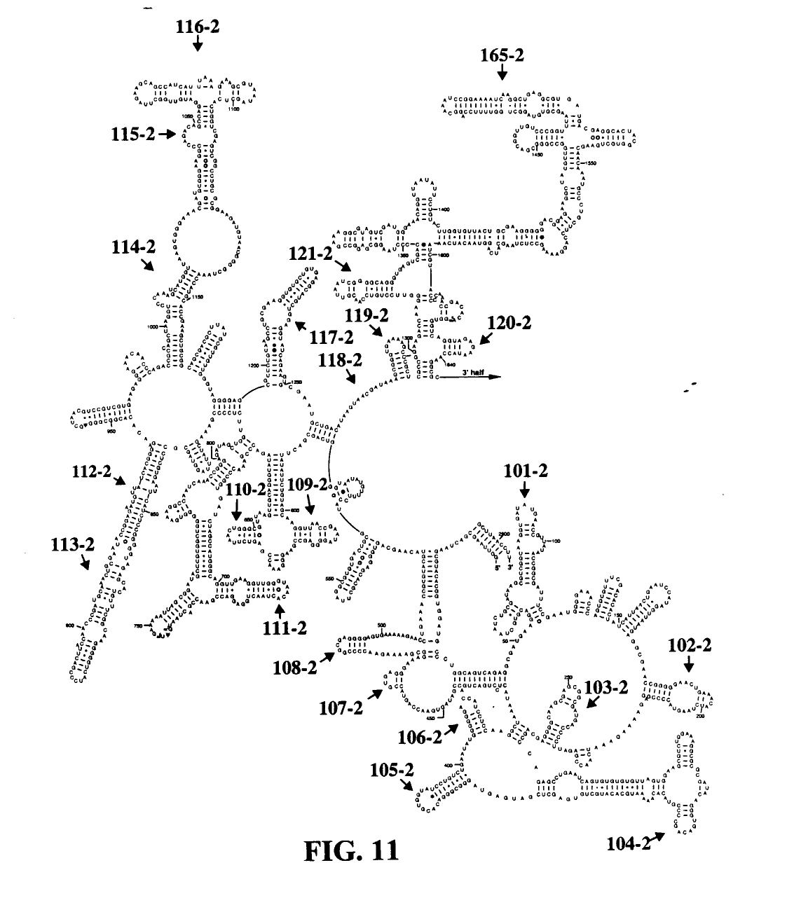

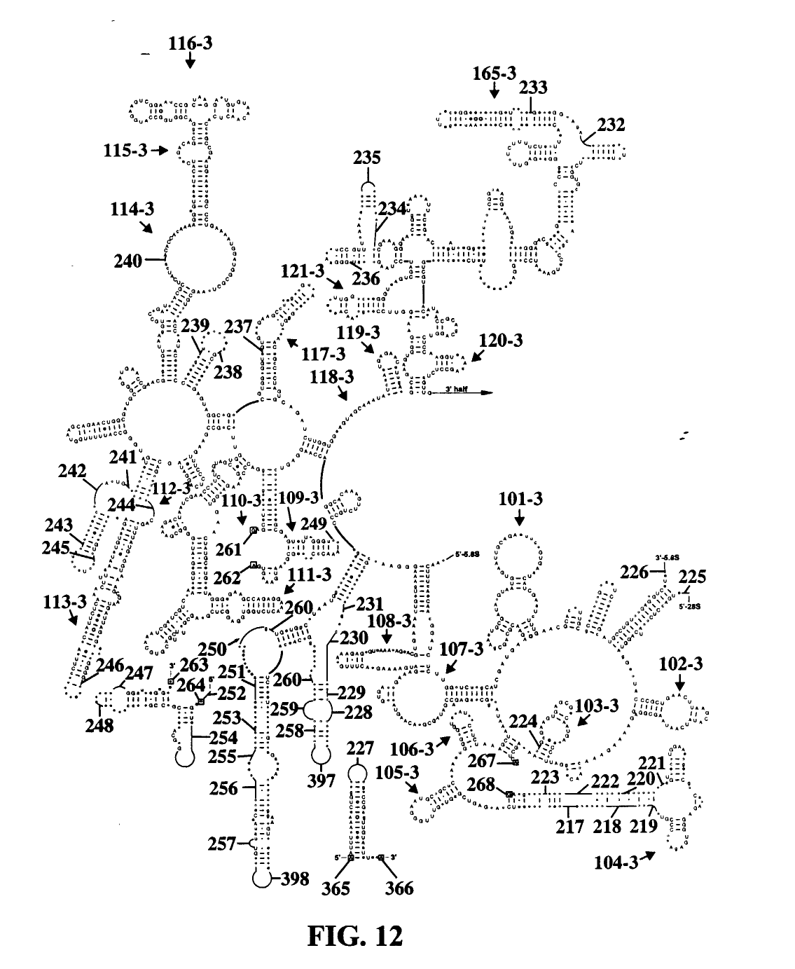

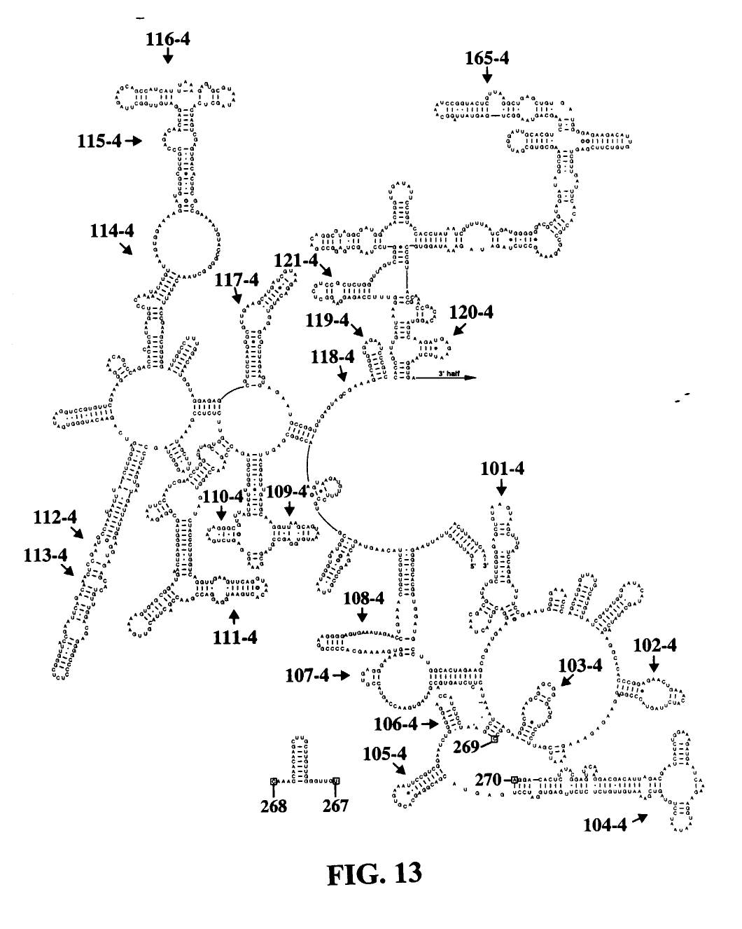

Additional molecular interaction sites can be located in numerous species of 23S rRNA. In the particular examples disclosed herein below, “n” refers to any nucleotide. For example, molecular interaction sites, in locations that correspond to those described above, can be found in the 23S rRNA of Candida albicans (SEQ ID NO:208) (FIG. 7), Archaea consensus (SEQ ID NO:209) (FIG. 8), Haloarcula marismortui (SEQ ID NO:210) (FIG. 9), chloroplast (SEQ ID NO:211) (FIG. 10), Escherichia coli (SEQ ID NO:212) (FIG. 11), fungal consensus (SEQ ID NO:213) (FIG. 12), and Staphylococcus aureus (SEQ ID NO:214) (FIG. 13).

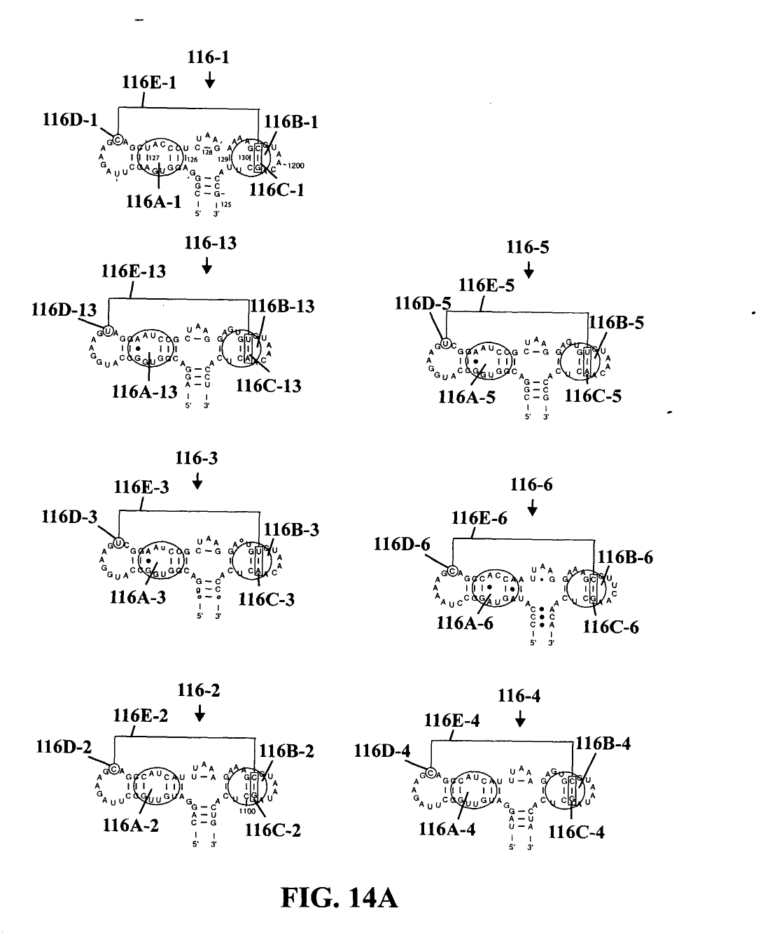

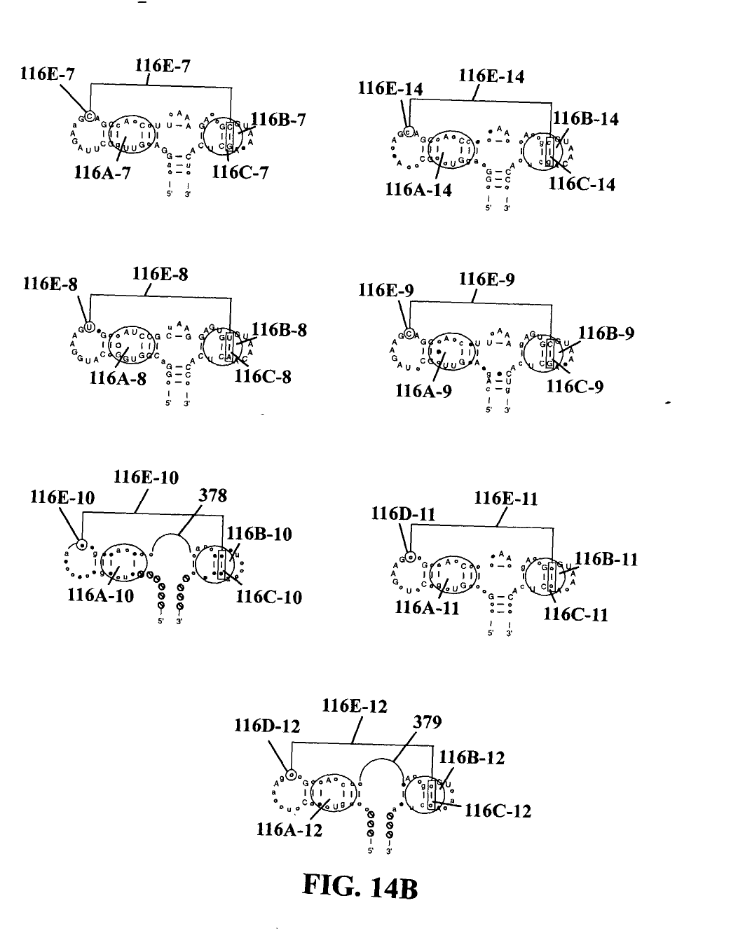

In particular, the following molecular interaction sites have been discovered. In some embodiments, molecular interaction site 16 comprises the sequence 5′-cgggaggugagcuuagaagcagcuacccucuaagaaaagcguaacagcuuaccg-3′ (SEQ ID NO:215) (Haloarcula marismortui), 5′-aggacgguggccauggaaguaggaauccgcuaaggaguguguaacaa cucaccu-3′ (SEQ ID NO:216) (Homo sapien), 5′-nggacgguggccauggaagucggaauccgcu aagganuguguaacaacucaccn-3′ (SEQ ID NO:217) (fungal consensus), 5′-caggauguuggcuuagaagcagccaucauuuaaagaaagcguaauagcucacug-3′ (SEQ ID NO:218) (Escherichia coli), 5′-cggacgguggccauggaagucggaauccgcuaaggaguguguaacaacucaccg-3′ (SEQ ID NO:219) (Candida albicans), 5′-cccauaguaggccuaaaagcagccaccaauuaagg aaagcguucaagcucaaca-3′ (SEQ ID NO:220) (Homo sapien mitochondria), 5′-uaggauguuggcuuagaagcagccaucauuuaaagagugcguaauagcucacua-3′ (SEQ ID NO:221) (Staphylococcus aureus), 5′-nagganguuggcuuagaagcagccancnuunaaaganngcguaanagc ucacun-3′ (SEQ ID NO:222) (bacterial consensus), 5′-nggacgguggncauggaagungnnau ccgcuaaggaguguguaacaacucaccn-3′ (SEQ ID NO:223) (eukaryote consensus), 5′-nnnnnnnunngnnnnnnanngnnannnnnannnnnunnnannnnnnnn-3′ (SEQ ID NO:224) (mitochondria), 5′-nggnaggunngcnnannagcagcnanccnnnaannanngcguaacagcunaccn-3′ (SEQ ID NO:225) (Archaea consensus), 5′-cagnanguungcnuagaagcagcnancnuunaaaga gugcguaanagcucacug-3′ (SEQ ID NO:226) (chloroplast), 5′-nngnnngungncnungaagnn gnnancnnnnaanganngnguaanancucacnn-3′ (SEQ ID NO:227) (three phylogenetic domains) or 5′-nnnnnngunnncnunnaagnngnnancnnnanngngunanancunannn-3′ (SEQ ID NO:228) (three phylogentic domains, chloroplast and mitochondria), each of which is shown as region 116 in FIGS. 14A and 14B. The conserved natures of each of the last two molecular interaction sites demonstrates the ability of the methods of the present inventions to be used to identify binding pockets across many species due to the underlying secondary structure.

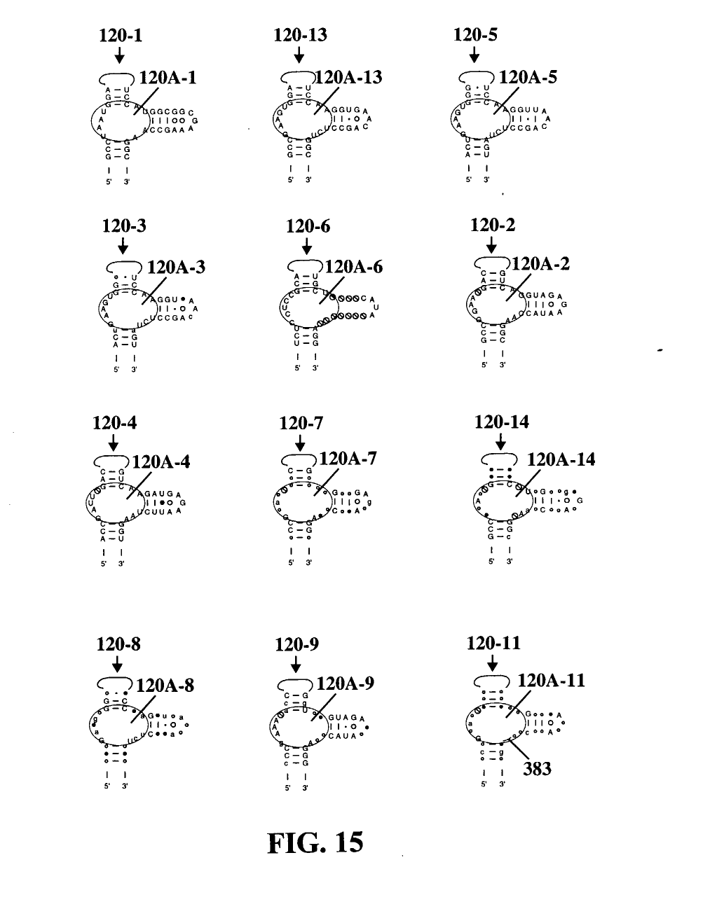

In some embodiments, molecular interaction site 20 comprises two polynucleotides, the first polynucleotide comprises the sequence 5′-gccuaaugga-3′ (SEQ ID NO:229) and the second polynucleotide comprises the sequence 5′-uccauggcggcgaaagccaaggc-3′ (SEQ ID NO:230) (Haloarcula marismortui), the first polynucleotide comprises the sequence 5′-acugaaguggn-3′ (SEQ ID NO:231) and the second polynucleotide comprises the sequence 5′-uccaaggunaacagccucuagu-3′ (SEQ ID NO:232) (fungal consensus), the first polynucleotide comprises the sequence 5′-accgauunggac-3′ (SEQ ID NO:233) and the second polynucleotide comprises the sequence 5′-gucaagaugagaauucuaaggu-3′ (SEQ ID NO:234) (Staphylococcus aureus), the first polynucleotide comprises the sequence 5′-nnngangnggn-3′ (SEQ ID NO:235) and the second polynucleotide comprises the sequence 5′-nccnagnunannanncucunnn-3′ (SEQ ID NO:236) (eukaryote consensus), the first polynucleotide comprises the sequence 5′-gccgaagugga-3′ (SEQ ID NO:237) and the second polynucleotide comprises the sequence 5′-uccaaggugaacagccucuggc-3′ (SEQ ID NO:238) (Homo sapien), the first polynucleotide comprises the sequence 5′-ucuccuccgca-3′ (SEQ ID NO:239) and the second polynucleotide comprises the sequence 5′-ugcunnnncauannnnnnnagg-3′ (SEQ ID NO:240) (Homo sapien mitochondria), the first polynucleotide comprises the sequence 5′-nccgnannnnc-3′ (SEQ ID NO:241) and the second polynucleotide comprises the sequence 5′-gnnnngnngagnanncnnaggn-3′ (SEQ ID NO:242) (bacterial consensus), the first polynucleotide comprises the sequence 5′-cccgaaanacc-3′ (SEQ ID NO:243) and the second polynucleotide comprises the sequence 5′-ggunnguagannauacnnaggg-3′ (SEQ ID NO:244) (chloroplast), the first polynucleotide comprises the sequence 5′-acugaaguggg-3′ (SEQ ID NO:245) and the second polynucleotide comprises the sequence 5′-uccaagguuaacagccucuagu-3′ (SEQ ID NO:246) (Candida albicans), the first polynucleotide comprises the sequence 5′-gccggaangac-3′ (SEQ ID NO:247) and the second polynucleotide comprises the sequence 5′-gucagguagagaauaccaaggc-3′ (SEQ ID NO:248) (Escherichia coli), the first polynucleotide comprises the sequence 5′-gccnnanngnn-3′ (SEQ ID NO:249) and the second polynucleotide comprises the sequence 5′-nncnungnngngnanncnaanggc-3′ (SEQ ID NO:250) (Archaea consensus), or the first polynucleotide comprises the sequence 5′-ncngnannnnn-3′ (SEQ ID NO:251) and the second polynucleotide comprises the sequence 5′-nnnnngnnnannanncnnngn-3′ (SEQ ID NO:252) (three phylogenetic domains), each of which is shown as region 120 in FIG. 15.

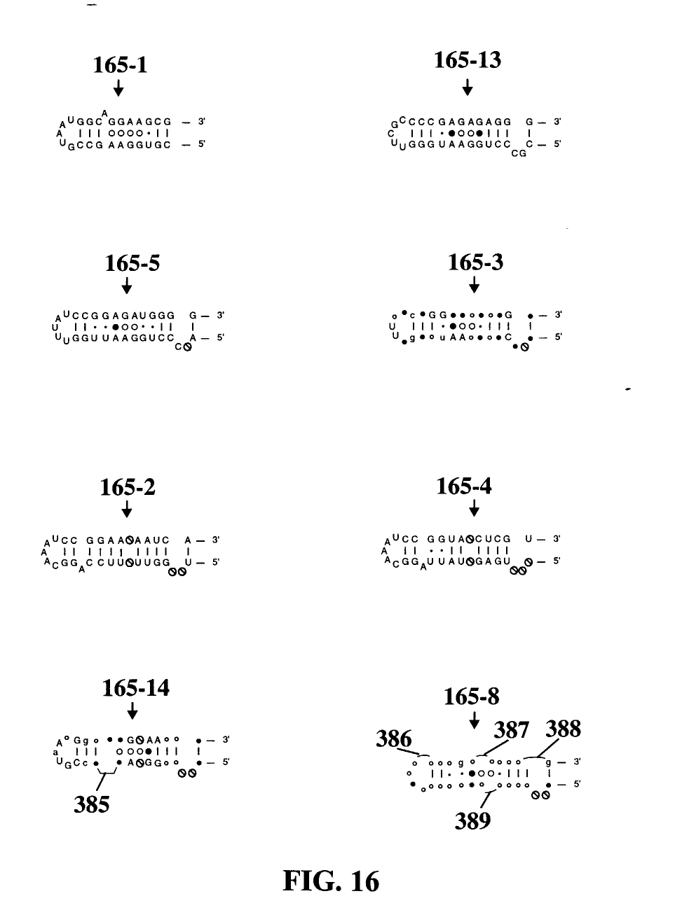

In some embodiments, molecular interaction site 36 comprises the sequence 5′-cguggaagccguaauggcaggaagcg-3′ (SEQ ID NO:253) (Haloarcula marismortui), 5′-ancccuggaauugguuuauccggagaugggg-3′ (SEQ ID NO:254) (Candida albicans), 5′-unngguunuuccaggcaaauccggaanaauca-3′ (SEQ ID NO:255) (Escherichia coli), 5′-nnnnnggnannccguaanggnnngnaannn-3′ (SEQ ID NO:256) (Archaea consensus), 5′-cgcccuggaauggguucgccccgagagaggg-3′ (SEQ ID NO:257) (Homo sapien), 5′-nnncnnnnaaunngnuunncnggnnnnnngn-3′ (SEQ ID NO:258) (fungal consensus), 5′-nnnugagnuauuaggcaaauccgguancucgu-3′ (SEQ ID NO:259) (Staphylococcus aureus), or 5′-nnnnnnnnnnnnnnnnnnnngnnnnng-3′ (SEQ ID NO:260) (eukaryote consensus), each of which is shown as region 165 in FIG. 16.

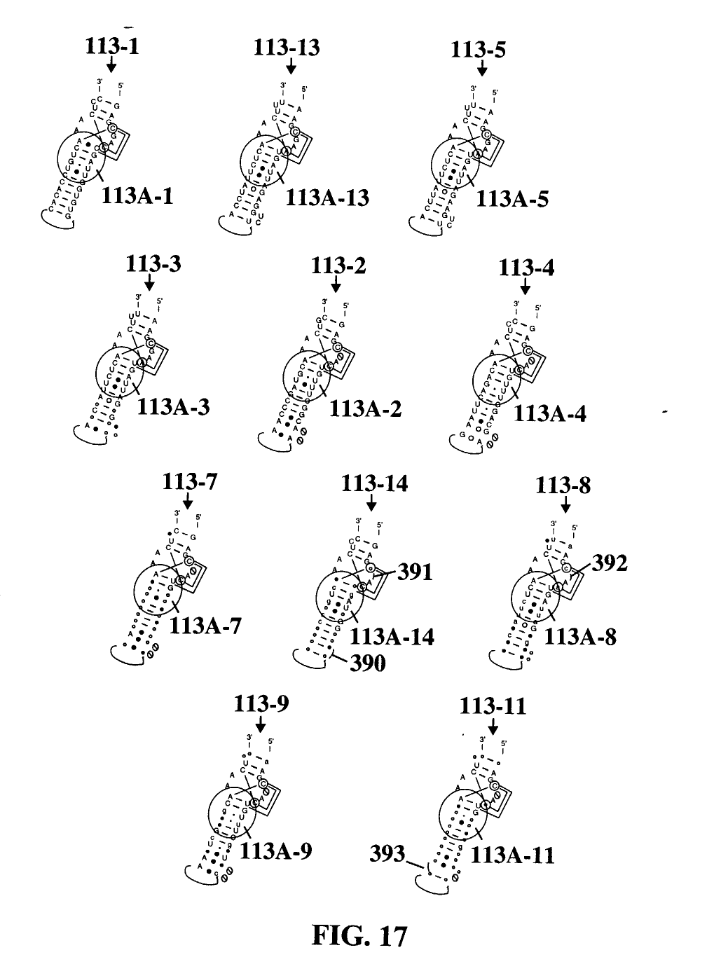

In some embodiments, molecular interaction site 13 comprises two polynucleotides, the first polynucleotide comprises the sequence 5′-gagcgaccgauuggugug-3′ (SEQ ID NO:261) and the second polynucleotide comprises the sequence 5′-cacaccugucaaacucc-3′ (SEQ ID NO:262) (Haloarcula marismortui), the first polynucleotide comprises the sequence 5′-aagcgaaugauuaga ggucu-3′ (SEQ ID NO:263) and the second polynucleotide comprises the sequence 5′-accuauucucaaacuuu-3′ (SEQ ID NO:264) (Homo sapien), the first polynucleotide comprises the sequence 5′-aagcgaaugauuagaagucu-3′ (SEQ ID NO:265) and the second polynucleotide comprises the sequence 5′-acuuauucucaaacuuu-3′ (SEQ ID NO:266) (Candida albicans), the first polynucleotide comprises the sequence 5′-aagcgaaugauuagngnnnn-3′ (SEQ ID NO:267) and the second polynucleotide comprises the sequence 5′-ancnauucucaaacuuu-3′ (SEQ ID NO:268) (fungal consensus), the first polynucleotide comprises the sequence 5′-gagcnacuguuucggcanna-3′ (SEQ ID NO:269) and the second polynucleotide comprises the sequence 5′-aacccgaugcaaacugc-3′ (SEQ ID NO:270) (Escherichia coli), the first polynucleotide comprises the sequence 5′-gagcnacuguuuggacgnna-3′ (SEQ ID NO:271) and the second polynucleotide comprises the sequence 5′-gaauucagacaaacucc-3′ (SEQ ID NO:272) (Staphylococcus aureus), the first polynucleotide comprises the sequence 5′-gagcnacugnnnnnnnnnnn-3′ (SEQ ID NO:273) and the second polynucleotide comprises the sequence 5′-nannnnnnnnaaacunc-3′ (SEQ ID NO:274) (bacterial consensus), the first polynucleotide comprises the sequence 5′-gagnacngaunggnnnn-3′ (SEQ ID NO:275) and the second polynucleotide comprises the sequence 5′-nnnnccngucnaacucc-3′ (SEQ ID NO:276) (Archaea consensus), the first polynucleotide comprises the sequence 5′-aagcaaugauuagngnnnn-3′ (SEQ ID NO:277) and the second polynucleotide comprises the sequence 5′-nncnauucucaaacunu-3′ (SEQ ID NO:278) (eukaryote consensus), the first polynucleotide comprises the sequence 5′-aagcnacuguuucgnunnnc-3′ (SEQ ID NO:279) and the second polynucleotide comprises the sequence 5′-aanucgnngcaaacunn-3′ (SEQ ID NO:280) (chloroplast), or the first polynucleotide comprises the sequence 5′-nagcnanugnnnngnnnnnn-3′ (SEQ ID NO:281) and the second polynucleotide comprises the sequence 5′-nnnnnnnnnnaaacunn-3′ (SEQ ID NO:282) (three phylogenetic domains), each of which is shown as region 113 in FIG. 17.

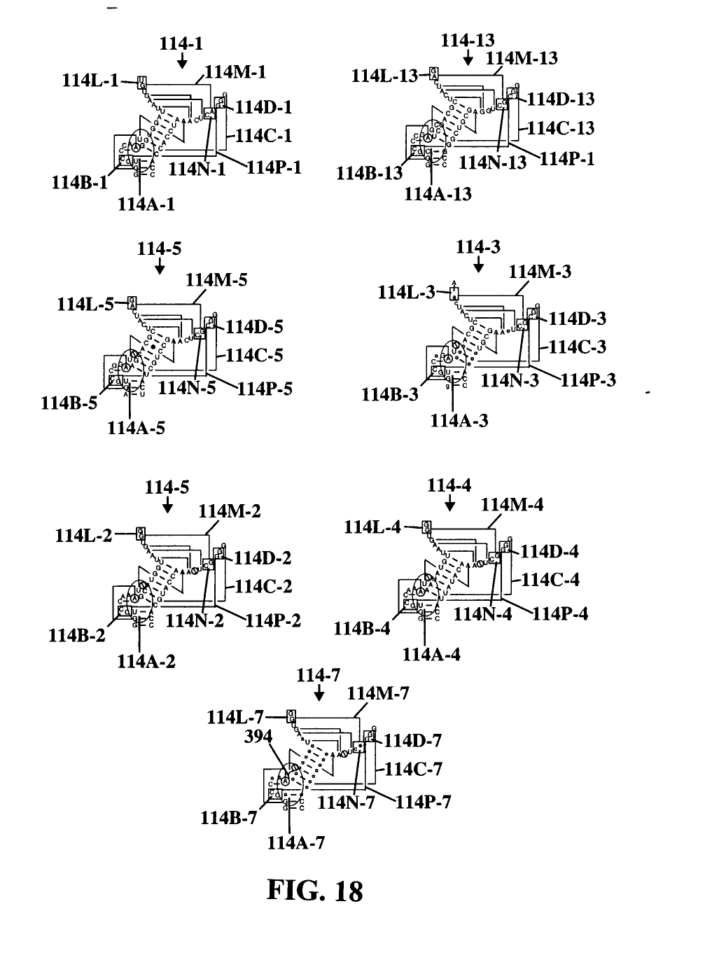

In some embodiments, molecular interaction site 14 comprises two polynucleotides, the first polynucleotide comprises the sequence 5′-gguccccaaguguggauuaagugu-3′ (SEQ ID NO:283) and the second polynucleotide comprises the sequence 5′-gggacucaaauccaccacc-3′ (SEQ ID NO:284) (Haloarcula marismortui), the first polynucleotide comprises the sequence 5′-agugccggaaugncacgcucaucag-3′ (SEQ ID NO:285) and the second polynucleotide comprises the sequence 5′-ggcgcucaagccugcuacu-3′ (SEQ ID NO:286) (Candida albicans), the first polynucleotide comprises the sequence 5′-ggucccaaagucnaugguuaagugg-3′ (SEQ ID NO:287) and the second polynucleotide comprises the sequence 5′-ggggcunaaaccuagcacc-3′ (SEQ ID NO:288) (Escherichia coli), the first polynucleotide comprises the sequence 5′-ggcgcccgaugccgacgcucaucag-3′ (SEQ ID NO:289) and the second polynucleotide comprises the sequence 5′-ggcgcuggagcgucgggcc-3′ (SEQ ID NO:290) (Homo sapien), the first polynucleotide comprises the sequence 5′-ggugccnganunnnacgcucaucaa-3′ (SEQ ID NO:291) and the second polynucleotide comprises the sequence 5′-ggcgcunaagcgunnnacc-3′ (SEQ ID NO:292) (fungal consensus), the first polynucleotide comprises the sequence 5′-ggucccaaaauanuauguuaagugg-3′ (SEQ ID NO:293) and the second polynucleotide comprises the sequence 5′-ggggcunaaacauauuacc-3′ (SEQ ID NO:294) (Staphylococcus aureus), or the first polynucleotide comprises the sequence 5′-ggncccnaannnnnnnnuaagugg-3′ (SEQ ID NO:295) and the second polynucleotide comprises the sequence 5′-gggncunaannnnnnnncc-3′ (SEQ ID NO:296) (bacterial consensus), each of which is shown as region 114 in FIG. 18.

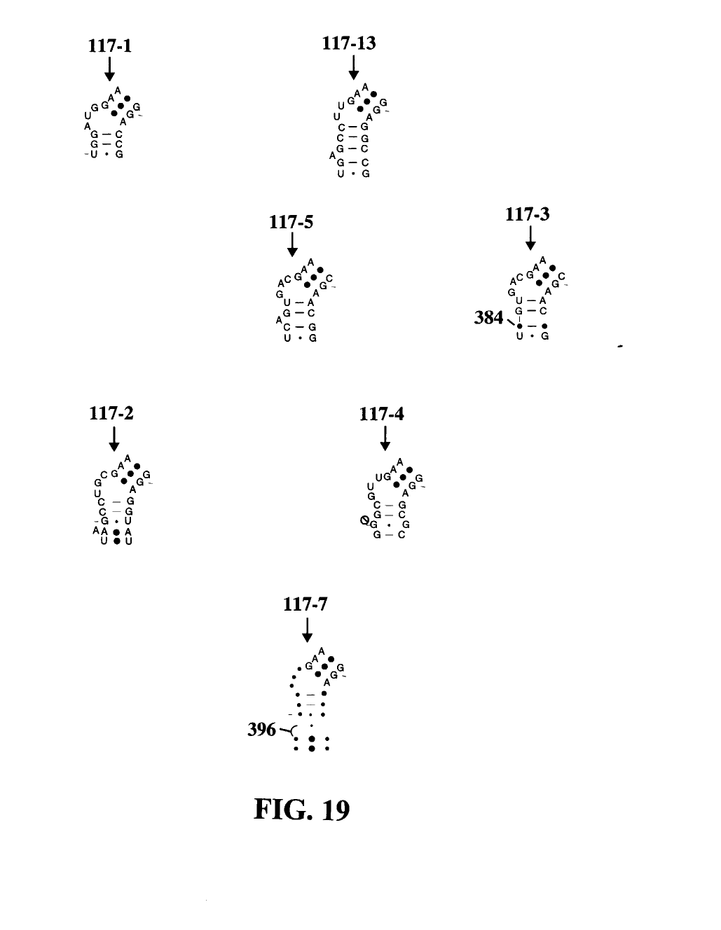

In some embodiments, molecular interaction site 17 comprises two polynucleotides, the first polynucleotide comprises the sequence 5′-uggauggaa-3′ and the second polynucleotide comprises the sequence 5′-ggaccg-3′ (Haloarcula marismortui), the first polynucleotide comprises the sequence 5′-ugagccuugaa-3′ (SEQ ID NO:297) and the second polynucleotide comprises the sequence 5′-ggaggccg-3′ (Homo sapien), the first polynucleotide comprises the sequence 5′-ucagugacgaa-3′ (SEQ ID NO:298) and the second polynucleotide comprises the sequence 5′-cgaacgg-3′ (Candida albicans), the first polynucleotide comprises the sequence 5′-ungugacgaa-3′ (SEQ ID NO:299) and the second polynucleotide comprises the sequence 5′-cgaacng-3′ (SEQ ID NO:300) (fungal consensus), the first polynucleotide comprises the sequence 5′-uaagccugcgaa-3′ (SEQ ID NO:301) and the second polynucleotide comprises the sequence 5′-ggagguau-3′ (SEQ ID NO:302) (Escherichia coli), the first polynucleotide comprises the sequence 5′-ggngcguugaa-3′ (SEQ ID NO:303) and the second polynucleotide comprises the sequence 5′-ggagcgc-3′ (Staphylococcus aureus), or the first polynucleotide comprises the sequence 5′-nnnnnnnngaa-3′ (SEQ ID NO:304) and the second polynucleotide comprises the sequence 5′-ggannnnn-3′ (bacterial consensus), each of which is shown as region 117 in FIG. 19.

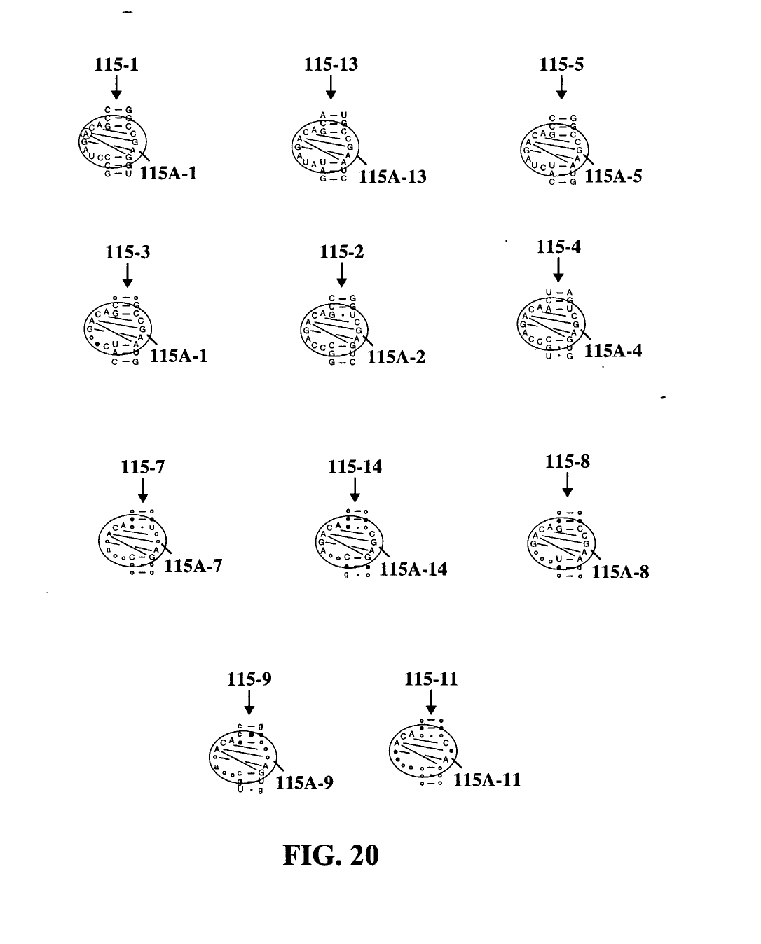

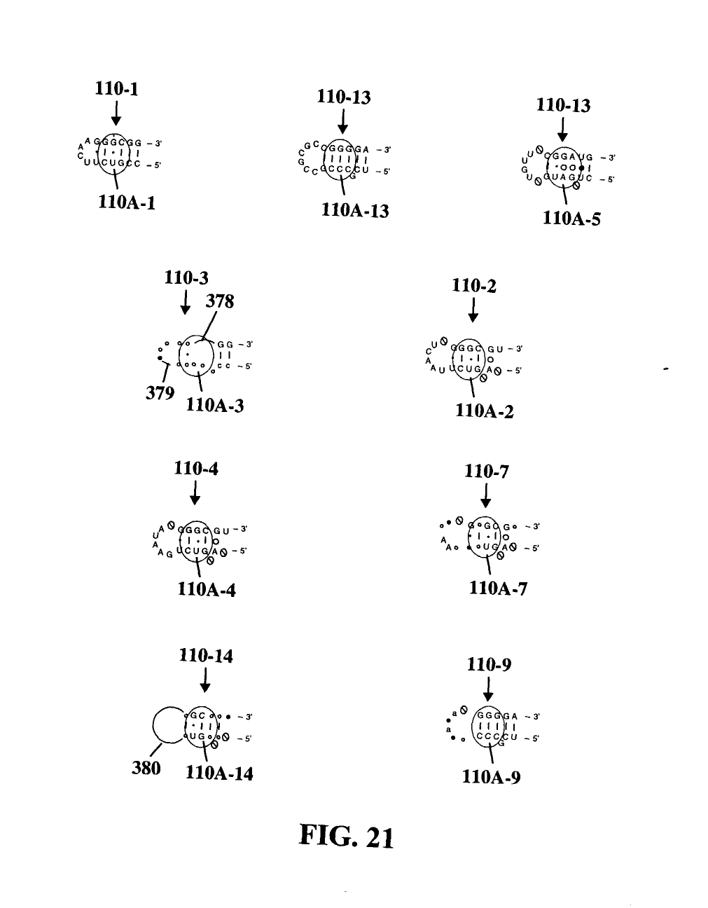

In some embodiments, molecular interaction site 15 comprises two polynucleotides, the first polynucleotide comprises the sequence 5′-gcccuagacagcc-3′ (SEQ ID NO:305) and the second polynucleotide comprises the sequence 5′-ggccgaggu-3′ (Haloarcula marismortui), the first polynucleotide comprises the sequence 5′-gauauagacagca-3′ (SEQ ID NO:306) and the second polynucleotide comprises the sequence 5′-ugccgaauc-3′ (Homo sapien), the first polynucleotide comprises the sequence 5′-caucuagacagcc-3′ (SEQ ID NO:307) and the second polynucleotide comprises the sequence 5′-ggccgaaug-3′ (Candida albicans), the first polynucleotide comprises the sequence 5′-caucnngacagcn-3′ (SEQ ID NO:308) and the second polynucleotide comprises the sequence 5′-ngccgaaug-3′ (fungal consensus), the first polynucleotide comprises the sequence 5′-ggcccagacagcc-3′ (SEQ ID NO:309) and the second polynucleotide comprises the sequence 5′-ggucgaguc-3′ (Escherichia coli), the first polynucleotide comprises the sequence 5′-ugcccagacaacu-3′ (SEQ ID NO:310) and the second polynucleotide comprises the sequence 5′-agucgagug-3′ (Staphylococcus aureus), the first polynucleotide comprises the sequence 5′-nncnnanacannn-3′ (SEQ ID NO:311) and the second polynucleotide comprises the sequence 5′-nnucnagnn-3′ (bacterial consensus), the first polynucleotide comprises the sequence 5′-gncnnagacannn-3′ (SEQ ID NO:312) and the second polynucleotide comprises the sequence 5′-nnncgagnn-3′ (Archaea consensus), the first polynucleotide comprises the sequence 5′-nnunnngacagnn-3′ (SEQ ID NO:313) and the second polynucleotide comprises the sequence 5′-nnccgaaun-3′ (SEQ ID NO:314) (eukaryote consensus), the first polynucleotide comprises the sequence 5′-ugcnnanacancc-3′ (SEQ ID NO:315) and the second polynucleotide comprises the sequence 5′-gnnnnagug-3′ (chloroplast), or the first polynucleotide comprises the sequence 5′-nnnnnnnacannn-3′ (SEQ ID NO:316) and the second polynucleotide comprises the sequence 5′-nnncnannn-3′ (three phylogenetic domains), each of which is shown as region 115 in FIG. 20.

In some embodiments, molecular interaction site 10 comprises the sequence 5′-ccgucuucaagggcgg-3′ (SEQ ID NO:317) (Haloarcula marismortui), 5′-ucgcccgccgcgccgggga-3′ (SEQ ID NO:318) (Homo sapien), 5′-cungaugnuguuncggaug-3′ (SEQ ID NO:319) (Candida albicans), 5′-ccnnnnnnnnnngg-3′ (SEQ ID NO:320) (fungal consensus), 5′-nangucuuaacungggcgu-3′ (SEQ ID NO:321) (Escherichia coli), 5′-nangucugaauangggcgu-3′ (SEQ ID NO:322) (Staphylococcus aureus), 5′-nangunnnaannngngcgn-3′ (SEQ ID NO:323) (bacterial consensus), 5′-nnnngunngcnnn-3′ (SEQ ID NO:324) (Archaea consensus), or 5′-ucgcccnnanangggga-3′ (SEQ ID NO:325) (chloroplast).

What is claimed is:

1. A polynucleotide comprising from about thirty five nucleotides to about one hundred forty nine nucleotides comprising a secondary structure defined by: a first side of a first stem comprising from about two nucleotides to about six nucleotides, a first side of a second stem comprising from about two nucleotides to about five nucleotides, a first terminal loop comprising from about four nucleotides to about twelve nucleotides, a second side of the second stem comprising from about two nucleotides to about five nucleotides, a first side of a first internal loop comprising from about three nucleotides to about seven nucleotides, a first side of a third stem comprising from about five nucleotides to about fifteen nucleotides wherein a first side of a second internal loop comprising from about one nucleotide to about three nucleotides is present in the first side of the third stem, a second terminal loop comprising from about four nucleotides to about ten nucleotides, a second side of the third stem comprising from about five nucleotides to about fifteen nucleotides wherein a second side of a second internal loop comprising from about three nucleotides to about nine nucleotides is present in the second side of the third stem, a second side of the first internal loop comprising from about two nucleotides to about five nucleotides, and a second side of the first stem comprising from about two nucleotides to about six nucleotides.

2. The polynucleotide of claim 1 comprising at least sixty four or sixty five nucleotides and up to one hundred fourteen or one hundred fifteen nucleotides comprising a secondary structure defined by: a first side of a first stem comprising four nucleotides, a first side of a second stem comprising three nucleotides, a first terminal loop comprising eight nucleotides, a second side of the second stem comprising three nucleotides, a first side of a first internal loop comprising five nucleotides, a first side of a third stem comprising ten nucleotides wherein a first side of a second internal loop comprising two nucleotides is present between the seventh and eighth nucleotides of the first side of the third stem, a second terminal loop comprising seven nucleotides, a second side of the third stem comprising ten nucleotides wherein a second side of a second internal loop comprising five or six nucleotides is present between the third and fourth nucleotides of the second side of the third stem, a second side of the first internal loop comprising three nucleotides, and a second side of the first stem comprising four nucleotides.

3. The polynucleotide of claim 2 comprising SEQ ID NO:1 or SEQ ID NO:2.

4. A polynucleotide comprising from about fourteen nucleotides to about eighty six nucleotides comprising a secondary structure defined by: a first side of a first stem comprising from about three nucleotides to about seven nucleotides wherein a first side of an internal loop comprising from about three nucleotides to about seven nucleotides is present in the first side of the stem, a terminal loop comprising from about two nucleotides to about six nucleotides, and a second side of the stem comprising from about three nucleotides to about seven nucleotides wherein a second side of the internal loop comprising from about three nucleotides to about nine nucleotides is present in the second side of the stem.

5. The polynucleotide of claim 4 comprising at least twenty five nucleotides and up to seventy five nucleotides comprising a secondary structure defined by: a first side of a first stem comprising five nucleotides wherein a first side of an internal loop comprising five nucleotides is present between the third and fourth nucleotides of the first side of the stem, a terminal loop comprising four nucleotides, and a second side of the stem comprising five nucleotides wherein a second side of the internal loop comprising six nucleotides is present between the second and third nucleotides of the second side of the stem.

6. The polynucleotide of claim 5 comprising SEQ ID NO:3.

7. A polynucleotide comprising from about twelve nucleotides to about eighty one nucleotides comprising a secondary structure defined by: a first side of a stem comprising from about two nucleotides to about six nucleotides wherein a first side of an internal loop comprising from about three nucleotides to about seven nucleotides is present in the first side of the stem, a terminal loop comprising from about three nucleotides to about seven nucleotides, and a second side of the stem comprising from about two nucleotides to about six nucleotides wherein a second side of the internal loop comprising from about two nucleotides to about five or four nucleotides is present in the second side of the stem.

8. The polynucleotide of claim 7 comprising at least twenty one or twenty two nucleotides and up to seventy one or seventy two nucleotides comprising a secondary structure defined by: a first side of a stem comprising four nucleotides wherein a first side of an internal loop comprising five nucleotides is present between the second and third nucleotides of the first side of the stem, a terminal loop comprising five nucleotides, and a second side of the stem comprising four nucleotides wherein a second side of the internal loop comprising three or four nucleotides is present between the second and third nucleotides of the second side of the stem.

9. The polynucleotide of claim 8 comprising SEQ ID NO:4 or SEQ ID NO:5.

10. A polynucleotide comprising from about thirty one nucleotides to about one hundred twenty seven nucleotides comprising a secondary structure defined by: a first side of a first stem comprising from about two nucleotides to about five nucleotides, a first side of a first internal loop comprising from about two nucleotides to about five nucleotides, a first side of a second stem comprising from about three nucleotides to about seven nucleotides, a first terminal loop comprising from about three nucleotides to about nine nucleotides, a second side of the second stem comprising from about three nucleotides to about seven nucleotides, a second side of the first internal loop comprising from about one nucleotide to about three nucleotides, a first side of a third stem comprising from about one nucleotide to about two nucleotides, a second terminal loop comprising from about two nucleotides to about five nucleotides, a second side of the third stem comprising from about one nucleotide to about two nucleotides, a first side of a second internal loop comprising from about one nucleotide to about two nucleotides, a first side of a fourth stem comprising from about two nucleotides to about five nucleotides, a third terminal loop comprising from about four nucleotides to about ten nucleotides, a second side of the fourth stem comprising from about two nucleotides to about five nucleotides, a second side of the second internal loop comprising from about two nucleotides to about five nucleotides, and a second side of the first stem comprising from about two nucleotides to about five nucleotides.

11. The polynucleotide of claim 10 comprising at least forty nine nucleotides and up to one hundred forty nine nucleotides comprising a secondary structure defined by: a first side of a first stem comprising three nucleotides, a first side of a first internal loop comprising three nucleotides, a first side of a second stem comprising five nucleotides, a first terminal loop comprising six nucleotides, a second side of the second stem comprising five nucleotides, a second side of the first internal loop comprising two nucleotides, a first side of a third stem comprising one nucleotide, a second terminal loop comprising three nucleotides, a second side of the third stem comprising one nucleotide, a first side of a second internal loop comprising one nucleotide, a first side of a fourth stem comprising three nucleotides, a third terminal loop comprising seven nucleotides, a second side of the fourth stem comprising three nucleotides, a second side of the second internal loop comprising three nucleotides, and a second side of the first stem comprising three nucleotides.

12. The polynucleotide of claim 11 comprising SEQ ID NO:6.

13. A polynucleotide comprising from about eight nucleotides to about seventy two nucleotides comprising a secondary structure defined by: a first side of a stem comprising from about two nucleotides to about five nucleotides, a terminal loop comprising from about four nucleotides to about twelve nucleotides, and a second side of the stem comprising from about two nucleotides to about five nucleotides.

14. The polynucleotide of claim 13 comprising at least fifteen nucleotides and up to one hundred fifteen nucleotides comprising a secondary structure defined by: a first side of a stem comprising three nucleotides, a terminal loop comprising eight nucleotides, and a second side of the stem comprising three nucleotides.

15. The polynucleotide of claim 14 comprising SEQ ID NO:7.

16. A polynucleotide comprising from about ten nucleotides to about seventy six nucleotides comprising a secondary structure defined by: a dangling region comprising from about one nucleotide to about three nucleotides, a first side of a stem comprising from about three nucleotides to about seven nucleotides, a terminal loop comprising from about three nucleotides to about nine nucleotides, and a second side of the stem comprising from about three nucleotides to about seven nucleotides.

17. The polynucleotide of claim 16 comprising at least eighteen nucleotides and up to sixty eight nucleotides comprising a secondary structure defined by: a dangling region comprising two nucleotides, a first side of a stem comprising five nucleotides, a terminal loop comprising six nucleotides, and a second side of the stem comprising five nucleotides.

18. The polynucleotide of claim 17 comprising SEQ ID NO:8.

19. A composition comprising a first polynucleotide and a second polynucleotide wherein:
the first polynucleotide comprises from about fifteen nucleotides to about forty eight nucleotides comprising a secondary structure defined by: a first side of a first stem comprising from about one nucleotide to about three nucleotides, a first side of an internal loop comprising from about seven nucleotides to about twenty four nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about two nucleotides to about six nucleotides, and a first side of a third stem comprising from about one nucleotide to about three nucleotides; and
the second polynucleotide comprises from about three nucleotides to about nine nucleotides and interacts with the first polynucleotide such that the two most 5′ nucleotides form the second side of the third stem and the two most 3′ nucleotides form the second side of the first stem.

20. The composition of claim 19 wherein the first polynucleotide comprises at least thirty to thirty two nucleotides but not more than eighty to eighty two nucleotides and comprises a secondary structure defined by: a first side of a first stem comprising two nucleotides, a first side of an internal loop comprising from fourteen to sixteen nucleotides, a first side of a second stem comprising two nucleotides, a terminal loop comprising four nucleotides, a second side of the second stem comprising two nucleotides, a second side of the internal loop comprising four nucleotides, and a first side of a third stem comprising two nucleotides; and
the second polynucleotide comprises at least six nucleotides but not more than fifty six nucleotides and interacts with the first polynucleotide such that the two most 5′ nucleotides form a second side of the third stem and the two most 3′ nucleotides form a second side of the first stem.

21. The composition of claim 20 wherein the first polynucleotide comprises SEQ ID NO:9, SEQ ID NO:10 or SEQ ID NO:11.

22. The composition of claim 20 wherein the second polynucleotide comprises 5′-ccungg-3′.

23. A polynucleotide comprising from about eighteen nucleotides to about ninety nine nucleotides comprising a secondary structure defined by: a first dangling region comprising from about five nucleotides to about thirteen nucleotides, a first side of a stem comprising from about two nucleotides to about six nucleotides, a terminal loop comprising from about two nucleotides to about six or five nucleotides, a second side of the stem comprising from about two nucleotides to about six nucleotides, and a second dangling region comprising from about seven nucleotides to about nineteen nucleotides.

24. The polynucleotide of claim 23 comprising at least thirty four or thirty five nucleotides and up to eighty four or eighty five nucleotides comprising a secondary structure defined by: a first dangling region comprising nine nucleotides, a first side of a stem comprising four nucleotides, a terminal loop comprising four or five nucleotides, a second side of the stem comprising four nucleotides, and a second dangling region comprising thirteen nucleotides.

25. The polynucleotide of claim 24 comprising SEQ ID NO:12 or SEQ ID NO:13.

26. A polynucleotide comprising from about ten nucleotides to about eighty six nucleotides comprising a secondary structure defined by: a first side of a first stem comprising from about two nucleotides to about six nucleotides, a first side of an internal loop comprising from about one nucleotide to about three nucleotides, a first side of a second stem comprising from about one nucleotide to about three nucleotides, a terminal loop comprising from about two nucleotides to about twelve nucleotides, a second side of the second stem comprising from about one nucleotide to about three nucleotides, a second side of the internal loop comprising from about one nucleotide to about three nucleotides, and a second side of the first stem comprising from about two nucleotides to about six nucleotides.

27. The polynucleotide of claim 26 comprising at least twenty to twenty four nucleotides and up to seventy to seventy four nucleotides comprising a secondary structure defined by: a first side of a first stem comprising four nucleotides, a first side of an internal loop comprising two nucleotides, a first side of a second stem comprising two nucleotides, a terminal loop comprising from four to eight nucleotides, a second side of the second stem comprising two nucleotides, a second side of the internal loop comprising two nucleotides, and a second side of the first stem comprising four nucleotides.

28. The polynucleotide of claim 27 comprising any one of SEQ ID NO:14 to SEQ ID NO:18.

29. A polynucleotide comprising from about nine nucleotides to about seventy three nucleotides comprising a secondary structure defined by: a dangling region comprising from about one nucleotide to about two nucleotides, a first side of a stem comprising from about two nucleotides to about six nucleotides, a terminal loop comprising from about three nucleotides to about seven nucleotides, a second side of the stem comprising from about two nucleotides to about six nucleotides, and a dangling region comprising from about one nucleotide to about two nucleotides.

30. The polynucleotide of claim 29 comprising at least sixteen nucleotides and up to sixty six nucleotides comprising a secondary structure defined by: a dangling region comprising one nucleotide, a first side of a stem comprising four nucleotides, a termiinal loop comprising five nucleotides, a second side of the stem comprising four nucleotides, and a dangling region comprising one nucleotide.

31. The polynucleotide of claim 30 comprising SEQ ID NO:19.

32. A polynucleotide comprising from about seven nucleotides to about sixty nine nucleotides comprising a secondary structure defined by: a first side of a stem comprising from about two nucleotides to about five nucleotides, a terminal loop comprising from about three nucleotides to about nine nucleotides, and a second side of the stem comprising from about two nucleotides to about five nucleotides.

33. The polynucleotide of claim 32 comprising at least twelve nucleotides and up to sixty two nucleotides comprising a secondary structure defined by: a first side of a stem comprising three nucleotides, a terminal loop comprising six nucleotides, and a second side of the stem comprising three nucleotides.

34. The polynucleotide of claim 33 comprising SEQ ID NO:20.

35. A composition comprising a first polynucleotide and a second polynucleotide wherein:
the first polynucleotide comprises from about five nucleotides to about fourteen nucleotides comprising a secondary structure defined by: a first side of a stem comprising from about four nucleotides to about ten nucleotides wherein a first bulge comprising from about one nucleotides to about two to four nucleotides is present in the first side of the stem; and
the second polynucleotide comprises from about five nucleotides to about sixteen nucleotides comprising a secondary structure defined by: a second side of the stem comprising from about four nucleotides to about ten nucleotides wherein a second bulge comprising from about one nucleotide to about six nucleotides is optionally present in the second side of the stem.

36. The composition of claim 35 wherein the first polynucleotide comprises at least eight to eleven nucleotides but not more than fifty eight to sixty one nucleotides and comprises a secondary structure defined by: a first side of a stem comprising seven nucleotides wherein a first bulge comprising one to four nucleotides is present between the third and fourth nucleotides of the first side of the stem; and
the second polynucleotide comprises at least seven to twelve nucleotides but not more than fifty seven to sixty two nucleotides and comprises a secondary structure defined by: a second side of the stem comprising seven nucleotides wherein a second bulge comprising one to four nucleotides is optionally present between the third and fourth nucleotides of the second side of the stem.

37. The composition of claim 36 wherein the first polynucleotide comprises 5′-nnnnnnnn-3′, 5′-nnnnnnnnn-3′, SEQ ID NO:21 or SEQ ID NO:22.

38. The composition of claim 36 wherein the second polynucleotide comprises 5′-nnnnnnn-3′, 5′-nnnnnnnn-3′, 5′-nnnnnnnnn-3′, SEQ ID NO:23, SEQ ID NO:24 or SEQ ID NO:25.

39. A composition comprising a first polynucleotide and a second polynucleotide wherein:
the first polynucleotide comprises from about eleven nucleotides to about twenty eight nucleotides comprising a secondary structure defined by: a first side of a first stem comprising from about two nucleotides to about five nucleotides, a first side of a first internal loop comprising from about two nucleotides to about five nucleotides, a first side of a second stem comprising from about two nucleotides to about five nucleotides, a first side of a second internal loop comprising from about one nucleotide to about two nucleotides, a first side of a third stem comprising from about two nucleotides to about six nucleotides, and a dangling region comprising from about two nucleotides to about five nucleotides; and
the second polynucleotide comprises from about twelve nucleotides to about twenty eight nucleotides comprising a secondary structure defined by: a dangling region comprising from about two nucleotides to about five nucleotides, a second side of the third stem comprising from about two nucleotides to about six nucleotides, a second side of the second internal loop comprising from about one nucleotide to about two nucleotides, a second side of the second stem comprising from about two nucleotides to about five nucleotides, a second side of the first internal loop comprising from about one nucleotide to about three nucleotides, and a second side of the first stem comprising from about two nucleotides to about five nucleotides wherein a bulge comprising from about one nucleotide to about two nucleotides is present in the second side of the first stem.

40. The composition of claim 39 wherein the first polynucleotide comprises at least seventeen nucleotides but not more than sixty seven nucleotides and comprises a secondary structure defined by: a first side of a first stem comprising three nucleotides, a first side of a first internal loop comprising three nucleotides, a first side of a second stem comprising three nucleotides, a first side of a second internal loop comprising one nucleotide, a first side of a third stem comprising four nucleotides, and a dangling region comprising three nucleotides; and
the second polynucleotide comprises at least seventeen nucleotides but not more than sixty seven nucleotides and comprises a secondary structure defined by: a dangling region comprising three nucleotides, a second side of the third stem comprising four nucleotides, a second side of the second internal loop comprising one nucleotide, a second side of the second stem comprising three nucleotides, a second side of the first internal loop comprising two nucleotides, and a second side of the first stem comprising three nucleotides wherein a bulge comprising one nucleotide is present between the second and third nucleotides of the second side of the first stem.

41. The composition of claim 40 wherein the first polynucleotide comprises SEQ ID NO:26.

42. The composition of claim 40 wherein the second polynucleotide comprises SEQ ID NO:27.

43. A composition comprising a first polynucleotide and a second polynucleotide wherein:
the first polynucleotide comprises from about fourteen nucleotides to about thirty six nucleotides and comprises the following features (5′ to 3′): a first side of a first stem comprising from about two nucleotides to about five nucleotides, a bulge comprising from about three nucleotides to about nine nucleotides, a first side of a second stem comprising from about four nucleotides to about ten nucleotides wherein a bulge of from about one nucleotide to about two nucleotides is optionally present in the first side of the first stem, and a dangling region comprising from about four nucleotides to about ten nucleotides; and
the second polynucleotide comprises from about ten nucleotides to about twenty seven nucleotides comprising a secondary structure defined by: a dangling region comprising from about four nucleotides to about twelve nucleotides, a second side of the second stem comprising from about four nucleotides to about ten nucleotides, and a second side of the first stem comprising from about two nucleotides to about five nucleotides.

44. The composition of claim 43 wherein the first polynucleotide comprises at least twenty three or twenty four nucleotides but not more than seventy three or seventy four nucleotides and comprises a secondary structure defined by: a first side of a first stem comprising three nucleotides, a bulge comprising six nucleotides, a first side of a second stem comprising seven nucleotides wherein a bulge of one nucleotide is optionally present between the first and second nucleotides of the first side of the first stem, and a dangling region comprising seven nucleotides; and
the second polynucleotide comprises at least eighteen nucleotides but not more than sixty eight nucleotides and comprises a secondary structure defined by: a dangling region comprising eight nucleotides, a second side of the second stem comprising seven nucleotides, and a second side of the first stem comprising three nucleotides.

45. The composition of claim 44 wherein the first polynucleotide comprises SEQ ID NO:28 or SEQ ID NO:29.

46. The composition of claim 44 wherein the second polynucleotide comprises SEQ ID NO:30.

47. A composition comprising a first polynucleotide and a second polynucleotide wherein:
the first polynucleotide comprises from about eight nucleotides to about twenty nucleotides comprising a secondary structure defined by: a first side of a first stem comprising from about two nucleotides to about five nucleotides, a first side of an internal loop comprising from about four nucleotides to about ten nucleotides, and a first side of a second stem comprising from about two nucleotides to about five nucleotides; and
the second polynucleotide comprises from about six nucleotides to about fifteen nucleotides comprising a secondary structure defined by: a second side of the second stem comprising from about two nucleotides to about five nucleotides, a second side of the internal loop comprising from about two nucleotides to about five nucleotides, and a second side of the first stem comprising from about two nucleotides to about five nucleotides.

48. The composition of claim 47 wherein the first polynucleotide comprises at least thirteen nucleotides but not more than sixty three nucleotides and comprises a secondary structure defined by: a first side of a first stem comprising three nucleotides, a first side of an internal loop comprising seven nucleotides, and a first side of a second stem comprising three nucleotides; and
the second polynucleotide comprises at least nine nucleotides but not more than fifty nine nucleotides and comprises a secondary structure defined by: a second side of the second stem comprising three nucleotides, a second side of the internal loop comprising three nucleotides, and a second side of the first stem comprising three nucleotides.

49. The composition of claim 48 wherein the first polynucleotide comprises SEQ ID NO:31.

50. The composition of claim 48 wherein the second polynucleotide comprises 5′-nnucnagnn-3′.

51. A polynucleotide comprising from about thirty five nucleotides to about one hundred thirty eight nucleotides comprising a secondary structure defined by: a first side of a first stem comprising from about two nucleotides to about five nucleotides, a first bulge of from about one nucleotide to about two nucleotides, a first side of a second stem comprising from about two nucleotides to about five nucleotides, a first side of a first internal loop comprising from about one nucleotide to about three nucleotides, a first side of a third stem comprising from about two nucleotides to about five nucleotides, a first terminal loop comprising from about five nucleotides to about thirteen nucleotides, a second side of the third stem comprising from about two nucleotides to about five nucleotides, a second side of the first internal loop comprising from about one nucleotide to about three nucleotides, a second side of the second stem comprising from about two nucleotides to about five nucleotides, a first side of a fourth stem comprising from about one nucleotide to about two nucleotides, a second terminal loop comprising from about two nucleotides to about five nucleotides, a second side of the fourth stem comprising from about one nucleotide to about two nucleotides, a first side of a fifth stem comprising from about two nucleotides to about five nucleotide wherein a first side of a second internal loop comprising from about two nucleotides to about five nucleotides is present in the first side of the fifth stem, a third terminal loop comprising from about three nucleotides to about nine nucleotides, a second side of the fifth stem comprising from about two nucleotides to about five nucleotides wherein a second side of the second internal loop comprising from about one nucleotide to about two nucleotides is present in the second side of the fifth stem, a second bulge of from about one nucleotide to about two nucleotides, and a second side of the first stem comprising from about two nucleotides to about five nucleotides.

52. The polynucleotide of claim 51 comprising at least fifty four nucleotides and up to one hundred four nucleotides comprising a secondary structure defined by: a first side of a first stem comprising three nucleotides, a first bulge of one nucleotide, a first side of a second stem comprising three nucleotides, a first side of a first internal loop comprising two nucleotides, a first side of a third stem comprising three nucleotides, a first terminal loop comprising nine nucleotides, a second side of the third stem comprising three nucleotides, a second side of the first internal loop comprising two nucleotides, a second side of the second stem comprising three nucleotides, a first side of a fourth stem comprising one nucleotide, a second terminal loop comprising three nucleotides, a second side of the fourth stem comprising one nucleotide, a first side of a fifth stem comprising three nucleotide wherein a first side of a second internal loop comprising three nucleotides is present between the first and second nucleotides of the first side of the fifth stem, a third terminal loop comprising six nucleotides, a second side of the fifth stem comprising three nucleotides wherein a second side of the second internal loop comprising one nucleotide is present between the second and third nucleotides of the second side of the fifth stem, a second bulge of one nucleotide, and a second side of the first stem comprising three nucleotides.

53. The polynucleotide of claim 52 comprising SEQ ID NO:32.

54. A composition comprising a first polynucleotide and a second polynucleotide wherein:
the first polynucleotide comprises from about seven nucleotides to about one hundred sixty six nucleotides comprising a secondary structure defined by: a first side of a stem comprising from about three nucleotides to about seven nucleotides wherein a bulge comprising from about one nucleotide to about one hundred fifty nucleotides is optionally present in the first side of the stem, and a dangling region comprising from about three nucleotides to about nine nucleotides; and
the second polynucleotide comprises from about five nucleotides to about twelve nucleotides comprising a secondary structure defined by: a dangling region comprising from about two nucleotides to about five nucleotides and a second side of the stem comprising from about three nucleotides to about seven nucleotides.

55. The composition of claim 54 wherein the first polynucleotide comprises at least eleven to one hundred fourteen nucleotides but not more than sixty one nucleotides to one hundred sixty four nucleotides and comprises a secondary structure defined by: a first side of a stem comprising five nucleotides wherein a bulge comprising from one to one hundred three nucleotides is optionally present between the second and third nucleotides of the first side of the stem, and a dangling region comprising six nucleotides; and
the second polynucleotide comprises at least eight nucleotides but not more than fifty eight nucleotides and comprises a secondary structure defined by: a dangling region comprising three nucleotides and a second side of the stem comprising five nucleotides.

56. The composition of claim 55 wherein the first polynucleotide comprises any one of SEQ ID NO:33 to SEQ ID NO:136.

57. The composition of claim 55 wherein the second polynucleotide comprises 5′-ggannnnn-3′.

58. A composition comprising a first polynucleotide and a second polynucleotide wherein:
the first polynucleotide comprises from about six nucleotides to about seventeen nucleotides comprising a secondary structure defined by: a first side of a stem comprising from about four nucleotides to about twelve nucleotides wherein a bulge comprising from about one nucleotide to about two nucleotides is present in the first side of the stem, and a dangling region comprising from about one nucleotide to about three nucleotides; and
the second polynucleotide comprises from about eight nucleotides to about twelve nucleotides comprising a secondary structure defined by: a second side of the stem comprising from about four nucleotides to about twelve nucleotides.

59. The composition of claim 58 wherein the first polynucleotide comprises at least eleven nucleotides but not more than sixty one nucleotides and comprises a secondary structure defined by: a first side of a stem comprising eight nucleotides wherein a bulge comprising one nucleotide is present between the fourth and fifth nucleotides of the first side of the stem, and a dangling region comprising two nucleotides; and
the second polynucleotide comprises at least eight nucleotides but not more than fifty eight nucleotides and comprises a secondary structure defined by: a second side of the stem comprising eight nucleotides.

60. The composition of claim 59 wherein the first polynucleotide comprises SEQ ID NO:137.

61. The composition of claim 59 wherein the second polynucleotide comprises 5′-gngaanuu-3′.

62. A polynucleotide comprising from about eight nucleotides to about seventy two nucleotides comprising a secondary structure defined by: a first side of a stem comprising from about two nucleotides to about six nucleotides, a terminal loop comprising from about four nucleotides to about ten nucleotides, and a second side of the stem comprising from about two nucleotides to about six nucleotides.

63. The polynucleotide of claim 62 comprising at least fifteen nucleotides and up to sixty five nucleotides comprising a secondary structure defined by: a first side of a stem comprising four nucleotides, a terminal loop comprising seven nucleotides, and a second side of the stem comprising four nucleotides.

64. The polynucleotide of claim 63 comprising SEQ ID NO:138.

65. A composition comprising a first polynucleotide and a second polynucleotide wherein:
the first polynucleotide comprises from about six nucleotides to about sixteen nucleotides comprising a secondary structure defined by: a first side of a stem comprising from about two nucleotides to about five nucleotides, a bulge comprising from about two nucleotides to about six nucleotides, and a first side of a second stem comprising from about two nucleotides to about five nucleotides; and
the second polynucleotide comprises from about thirteen nucleotides to about thirty four nucleotides comprising a secondary structure defined by: a second side of the second stem comprising from about two nucleotides to about five nucleotides, a bulge comprising from about one nucleotide to about two nucleotides, a first side of a third stem comprising from about two nucleotides to about six nucleotides, a terminal loop comprising from about three nucleotides to about seven nucleotides, a second side of the third stem comprising from about two nucleotides to about six nucleotides, a bulge comprising from about one nucleotide to about three nucleotides, and a second side of the first stem comprising from about two nucleotides to about five nucleotides.

66. The composition of claim 65 wherein the first polynucleotide comprises at least ten nucleotides but not more than sixty nucleotides and comprises a secondary structure defined by: a first side of a stem comprising three nucleotides, a bulge comprising four nucleotides, and a first side of a second stem comprising three nucleotides; and
the second polynucleotide comprises at least twenty two nucleotides but not more than seventy two nucleotides and comprises a secondary structure defined by: a second side of the second stem comprising three nucleotides, a bulge comprising one nucleotide, a first side of a third stem comprising four nucleotides, a terminal loop comprising five nucleotides, a second side of the third stem comprising four nucleotides, a bulge comprising two nucleotides, and a second side of the first stem comprising three nucleotides.

67. The composition of claim 66 wherein the first polynucleotide comprises SEQ ID NO:139.

68. The composition of claim 66 wherein the second polynucleotide comprises SEQ ID NO:140.

69. A polynucleotide comprising from about eleven nucleotides to about seventy nine nucleotides comprising a secondary structure defined by: a first side of a first stem comprising from about two nucleotides to about five nucleotides, a first side of an internal loop comprising from about two nucleotides to about five nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about one nucleotide to about two nucleotides, and a second side of the first stem comprising from about two nucleotides to about five nucleotides.

70. The polynucleotide of claim 69 comprising at least nineteen nucleotides and up to sixty nine nucleotides comprising a secondary structure defined by: a first side of a first stem comprising three nucleotides, a first side of an internal loop comprising three nucleotides, a first side of a second stem comprising two nucleotides, a terminal loop comprising four nucleotides, a second side of the second stem comprising two nucleotides, a second side of the internal loop comprising one nucleotide, and a second side of the first stem comprising three nucleotides.

71. The polynucleotide of claim 70 comprising SEQ ID NO:141.

72. A composition comprising a first polynucleotide and a second polynucleotide wherein:
the first polynucleotide comprises from about six nucleotides to about sixteen nucleotides comprising a secondary structure defined by: a first side of a stem comprising from about two nucleotides to about five nucleotides, a first side of an internal loop comprising from about two nucleotides to about six nucleotides, and a first side of a second stem comprising from about two nucleotides to about five nucleotides; and
the second polynucleotide comprises from about five nucleotides to about twelve nucleotides comprising a secondary structure defined by: a second side of the second stem comprising from about two nucleotides to about five nucleotides, a second side of the internal loop comprising from about one nucleotide to about two nucleotides, and a second side of the first stem comprising from about two nucleotides to about five nucleotides.

73. The composition of claim 72 wherein the first polynucleotide comprises at least ten nucleotides but not more than sixty nucleotides and comprises a secondary structure defined by: a first side of a stem comprising three nucleotides, a first side of an internal loop comprising four nucleotides, and a first side of a second stem comprising three nucleotides; and
the second polynucleotide comprises at least seven nucleotides but not more than fifty seven nucleotides and comprises a secondary structure defined by: a second side of the second stem comprising three nucleotides, a second side of the internal loop comprising one nucleotide, and a second side of the first stem comprising three nucleotides.

74. The composition of claim 73 wherein the first polynucleotide comprises SEQ ID NO:142.

75. The composition of claim 73 wherein the second polynucleotide comprises 5′-nnnncnc-3′.

76. A polynucleotide comprising from about fifteen nucleotides to about eighty nine nucleotides comprising a secondary structure defined by: a dangling region comprising from about one nucleotide to about two nucleotides, a first side of a first stem comprising from about three nucleotides to about seven nucleotides, a first side of an internal loop comprising from about two nucleotides to about five nucleotides, a first side of a second stem comprising from about one nucleotide to about three nucleotides, a terminal loop comprising from about two nucleotides to about six nucleotides, a second side of the second stem comprising from about one nucleotide to about three nucleotides, a second side of the internal loop comprising from about two nucleotides to about six nucleotides, and a second side of the first stem comprising from about three nucleotides to about seven nucleotides.

77. The polynucleotide of claim 76 comprising at least twenty six nucleotides and up to seventy six nucleotides comprising a secondary structure defined by: a dangling region comprising one nucleotide, a first side of a first stem comprising five nucleotides, a first side of an internal loop comprising three nucleotides, a first side of a second stem comprising two nucleotides, a terminal loop comprising four nucleotides, a second side of the second stem comprising two nucleotides, a second side of the internal loop comprising four nucleotides, and a second side of the first stem comprising five nucleotides.

78. The polynucleotide of claim 77 comprising SEQ ID NO:143.

79. A composition comprising a first polynucleotide and a second polynucleotide wherein:
the first polynucleotide comprises from about four nucleotides to about twelve nucleotides comprising a secondary structure defined by: a first side of a stem comprising from about three nucleotides to about nine nucleotides wherein a first side of an internal loop comprising from about one nucleotide to about three nucleotides is present in the first side of the stem; and
the second polynucleotide comprises from about five nucleotides to about fifteen nucleotides comprising a secondary structure defined by: a second side of the stem comprising from about three nucleotides to about nine nucleotides wherein a second side of the internal loop comprising from about one nucleotide to about three nucleotides is present in the second side of the stem and wherein a bulge comprising from about one nucleotide to about three nucleotides is present in the second side of the stem.

80. The composition of claim 79 wherein the first polynucleotide comprises at least eight nucleotides but not more than fifty eight nucleotides and comprises a secondary structure defined by: a first side of a stem comprising six nucleotides wherein a first side of an internal loop comprising two nucleotides is present between the fourth and fifth nucleotides of the first side of the stem; and
the second polynucleotide comprises at least ten nucleotides but not more than sixty nucleotides and comprises a secondary structure defined by: a second side of the stem comprising six nucleotides wherein a second side of the internal loop comprising two nucleotides is present between the second and third nucleotides of the second side of the stem and wherein a bulge comprising two nucleotides is present between the third and fourth nucleotides of the second side of the stem.

81. The composition of claim 80 wherein the first polynucleotide comprises 5′-nugcnaan-3′.

82. The composition of claim 80 wherein the second polynucleotide comprises SEQ ID NO:144.

83. A composition comprising a first polynucleotide and a second polynucleotide wherein:
the first polynucleotide comprises from about eight nucleotides to about twenty one nucleotides comprising a secondary structure defined by: a first side of a first stem comprising from about two nucleotides to about six nucleotides, a bulge comprising from about one nucleotide to about two nucleotides, a first side of a second stem comprising from about four nucleotides to about ten nucleotides wherein a bulge comprising from about one nucleotide to about three nucleotides is present in the first side of the second stem; and
the second polynucleotide comprises from about seven nucleotides to about eighteen nucleotides comprising a secondary structure defined by: a second side of the second stem comprising from about four nucleotides to about ten nucleotides wherein a bulge comprising from about one nucleotide to about three nucleotides is present in the second side of the second stem, and a first side of a third stem comprising from about two nucleotides to about five nucleotides. The third polynucleotide comprises from about eight nucleotides to about twenty nucleotides comprising a secondary structure defined by: a second side of the third stem comprising from about two nucleotides to about five nucleotides, a first side of a fourth stem comprising from about one nucleotide to about two nucleotides, a terminal loop comprising from about two nucleotides to about five nucleotides, a second side of the fourth stem comprising from about one nucleotide to about two nucleotides, and a dangling region comprising from about two nucleotides to about six nucleotides. The fourth polynucleotide comprises from about five nucleotides to about thirteen nucleotides comprising a secondary structure defined by: a dangling region comprising from about three nucleotides to about seven nucleotides, and a second side of the first stem comprising from about two nucleotides to about six nucleotides.

84. The composition of claim 83 wherein the first polynucleotide comprises at least fourteen nucleotides but not more than sixty four nucleotides and comprises a secondary structure defined by: a first side of a first stem comprising four nucleotides, a bulge comprising one nucleotide, a first side of a second stem comprising seven nucleotides wherein a bulge comprising two nucleotides is present between the third and fourth nucleotides of the first side of the second stem;
the second polynucleotide comprises at least twelve nucleotides but not more than sixty two nucleotides and comprises a secondary structure defined by: a second side of the second stem comprising seven nucleotides wherein a bulge comprising two nucleotides is present between the second and third nucleotides of the second side of the second stem, and a first side of a third stem comprising three nucleotides;
the third polynucleotide comprises at least twelve nucleotides but not more than sixty two nucleotides and comprises a secondary structure defined by: a second side of the third stem comprising three nucleotides, a first side of a fourth stem comprising one nucleotide, a terminal loop comprising three nucleotides, a second side of the fourth stem comprising one nucleotide, and a dangling region comprising four nucleotides; and
the fourth polynucleotide comprises at least nine nucleotides but not more than fifty nine nucleotides and comprises a secondary structure defined by: a dangling region comprising five nucleotides, and a second side of the first stem comprising four nucleotides.

85. The composition of claim 84 wherein the first polynucleotide comprises SEQ ID NO:145.

86. The composition of claim 84 wherein the second polynucleotide comprises SEQ ID NO:146.

87. The composition of claim 84 wherein the third polynucleotide comprises SEQ ID NO:147.

88. The composition of claim 84 wherein the fourth polynucleotide comprises SEQ ID NO:148.

89. A composition comprising a first polynucleotide and a second polynucleotide wherein:
the first polynucleotide comprises from about eight nucleotides to about twenty nucleotides comprising a secondary structure defined by: a first side of a stem comprising from about five nucleotides to about thirteen nucleotides wherein a first side of a first internal loop comprising from about two nucleotides to about five nucleotides is present in the first side of the stem and wherein a first side of a second internal loop comprising from about one nucleotide to about two nucleotides is present in the first side of the stem; and
the second polynucleotide comprises from about eight nucleotides to about twenty nucleotides comprising a secondary structure defined by: a second side of the stem comprising from about five nucleotides to about thirteen nucleotides wherein a second side of the second internal loop comprising from about one nucleotide to about two nucleotides is present in the second side of the stem and wherein a second side of the first internal loop comprising from about two nucleotides to about five nucleotides is present in the second side of the stem.

90. The composition of claim 89 wherein the first polynucleotide comprises at least thirteen nucleotides but not more than sixty three nucleotides and comprises a secondary structure defined by: a first side of a stem comprising nine nucleotides wherein a first side of a first internal loop comprising three nucleotides is present between the second and third nucleotides of the first side of the stem and wherein a first side of a second internal loop comprising one nucleotide is present between the fourth and fifth nucleotides of the first side of the stem; and
the second polynucleotide comprises at least thirteen nucleotides but not more than sixty three nucleotides and comprises a secondary structure defined by: a second side of the stem comprising nine nucleotides wherein a second side of the second internal loop comprising one nucleotide is present between the fifth and sixth nucleotides of the second side of the stem and wherein a second side of the first internal loop comprising three nucleotides is present between the seventh and eighth nucleotides of the second side of the stem.

91. The composition of claim 90 wherein the first polynucleotide comprises SEQ ID NO:149.

92. The composition of claim 90 wherein the second polynucleotide comprises SEQ ID NO:150.

93. A polynucleotide comprising from about nine nucleotides to about seventy five nucleotides comprising a secondary structure defined by: a first side of a stem comprising from about three nucleotides to about nine nucleotides, a terminal loop comprising from about three nucleotides to about seven nucleotides, and a second side of the stem comprising from about three nucleotides to about nine nucleotides.

94. The polynucleotide of claim 93 comprising at least seventeen nucleotides and up to sixty seven nucleotides comprising a secondary structure defined by: a first side of a stem comprising six nucleotides, a terminal loop comprising five nucleotides, and a second side of the stem comprising six nucleotides.

95. The polynucleotide of claim 94 comprising SEQ ID NO:151.

96. A composition comprising a first polynucleotide and a second polynucleotide wherein:
the first polynucleotide comprises from about six nucleotides to about fifteen nucleotides comprising a secondary structure defined by: a first side of a first stem comprising from about two nucleotides to about five nucleotides, a bulge comprising from about one nucleotide to about three nucleotides, and a first side of a second stem comprising from about three nucleotides to about seven nucleotides;
the second polynucleotide comprises from about nine nucleotides to about twenty one nucleotides comprising a secondary structure defined by: a second side of the second stem comprising from about three nucleotides to about seven nucleotides, a first side of an internal loop comprising from about two nucleotides to about five nucleotides, and a first side of a third stem comprising from about three nucleotides to about seven nucleotides wherein a bulge comprising from about one nucleotide to about two nucleotides is optionally present in the first side of the third stem; and
the third polynucleotide comprises from about eight nucleotides to about nineteen nucleotides comprising a secondary structure defined by: a second side of the third stem comprising from about three nucleotides to about seven nucleotides, a second side of the internal loop comprising from about three nucleotides to about seven nucleotides, and a second side of the first stem comprising from about two nucleotides to about five nucleotides.

97. The composition of claim 96 wherein the first polynucleotide comprises at least ten nucleotides but not more than sixty nucleotides and comprises a secondary structure defined by: a first side of a first stem comprising three nucleotides, a bulge comprising two nucleotides, and a first side of a second stem comprising five nucleotides;
the second polynucleotide comprises at least thirteen or fourteen nucleotides but not more than sixty three or sixty four nucleotides and comprises a secondary structure defined by: a second side of the second stem comprising five nucleotides, a first side of an internal loop comprising three nucleotides, and a first side of a third stem comprising five nucleotides wherein a bulge comprising one nucleotide is optionally present between the third and fourth nucleotides of the first side of the third stem; and
the third polynucleotide comprises at least thirteen nucleotides but not more than sixty three nucleotides and comprises a secondary structure defined by: a second side of the third stem comprising five nucleotides, a second side of the internal loop comprising five nucleotides, and a second side of the first stem comprising three nucleotides.

98. The composition of claim 97 wherein the first polynucleotide comprises SEQ ID NO:152.

99. The composition of claim 97 wherein the second polynucleotide comprises SEQ ID NO:153 or SEQ ID NO:154.

100. The composition of claim 97 wherein the third polynucleotide comprises SEQ ID NO:155.

101. A composition comprising a first polynucleotide and a second polynucleotide wherein:
the first polynucleotide comprises from about thirteen nucleotides to about thirty four nucleotides comprising a secondary structure defined by: a first side of a first stem comprising from about two nucleotides to about six nucleotides, a bulge comprising from about five nucleotides to about thirteen nucleotides, a first side of a second stem comprising from about three nucleotides to about seven nucleotides, a first side of an internal loop comprising from about one nucleotide to about three nucleotides, and a first side of a third stem comprising from about two nucleotides to about five nucleotides; and
the second polynucleotide comprises from about thirteen nucleotides to about thirty six nucleotides comprising a secondary structure defined by: a second side of the third stem comprising from about two nucleotides to about five nucleotides, a second side of an internal loop comprising from about six nucleotides to about eighteen nucleotides, a second side of the second stem comprising from about three nucleotides to about seven nucleotides, and a second side of the first stem comprising from about two nucleotides to about six nucleotides.

102. The composition of claim 101 wherein the first polynucleotide comprises at least twenty three nucleotides but not more than seventy three nucleotides and comprises a secondary structure defined by: a first side of a first stem comprising four nucleotides, a bulge comprising nine nucleotides, a first side of a second stem comprising five nucleotides, a first side of an internal loop comprising two nucleotides, and a first side of a third stem comprising three nucleotides; and
the second polynucleotide comprises at least twenty four nucleotides but not more than seventy four nucleotides and comprises a secondary structure defined by: a second side of the third stem comprising three nucleotides, a second side of the internal loop comprising twelve nucleotides, a second side of the second stem comprising five nucleotides, and a second side of the first stem comprising four nucleotides.

103. The composition of claim 102 wherein the first polynucleotide comprises SEQ ID NO:156.

104. The composition of claim 102 wherein the second polynucleotide comprises SEQ ID NO:157.

105. A polynucleotide comprising from about nineteen nucleotides to about one hundred three nucleotides comprising a secondary structure defined by: a first side of a first stem comprising from about two nucleotides to about six nucleotides, a terminal loop comprising from about three nucleotides to about seven nucleotides, a second side of the first stem comprising from about two nucleotides to about six nucleotides, a first side of a second stem comprising from about three nucleotides to about nine nucleotides, a terminal loop comprising from about six nucleotides to about sixteen nucleotides, and a second side of the second stem comprising from about three nucleotides to about nine nucleotides.

106. The polynucleotide of claim 105 comprising at least thirty six nucleotides and up to eighty six nucleotides comprising a secondary structure defined by: a first side of a first stem comprising four nucleotides, a terminal loop comprising five nucleotides, a second side of the first stem comprising four nucleotides, a first side of a second stem comprising six nucleotides, a terminal loop comprising eleven nucleotides, and a second side of the second stem comprising six nucleotides.

107. The polynucleotide of claim 106 comprising SEQ ID NO:158.

108. A composition comprising a first polynucleotide and a second polynucleotide wherein:
the first polynucleotide comprises from about eleven nucleotides to about twenty nine nucleotides comprising a secondary structure defined by: a dangling region comprising from about one nucleotide to about two nucleotides, a first side of a first stem comprising from about one nucleotide to about three nucleotides, a bulge comprising from about two nucleotides to about six nucleotides, a first side of a second stem comprising from about three nucleotides to about nine nucleotides, and a first side of a third stem comprising from about three nucleotides to about seven nucleotides wherein a bulge comprising from about one nucleotide to about two nucleotides is present in the first side of the third stem:
the second polynucleotide comprises from about seventeen nucleotides to about forty eight nucleotides comprising a secondary structure defined by: a second side of the third stem comprising from about three nucleotides to about seven nucleotides wherein a bulge comprising from about two nucleotides to about five nucleotides is present in the second side of the third stem, a first side of a fourth stem comprising from about one nucleotide to about three nucleotides, a terminal loop comprising from about three nucleotides to about nine nucleotides, a second side of the fourth stem comprising from about one nucleotide to about three nucleotides, a bulge comprising from about two nucleotides to about six nucleotides, a second side of the second stem comprising from about three nucleotides to about nine nucleotides, and a dangling region comprising from about two nucleotides to about six nucleotides; and
the third polynucleotide comprises from about four nucleotides to about ten nucleotides comprising a secondary structure defined by: a second side of the first stem comprising from about one nucleotide to about three nucleotides and a dangling region comprising from about three nucleotides to about seven nucleotides.

109. The composition of claim 108 wherein the first polynucleotide comprises at least nineteen nucleotides but not more than sixty nine nucleotides and comprises a secondary structure defined by: a dangling region comprising one nucleotide, a first side of a first stem comprising two nucleotides, a bulge comprising four nucleotides, a first side of a second stem comprising six nucleotides, and a first side of a third stem comprising five nucleotides wherein a bulge comprising one nucleotide is present between the second and third nucleotides of the first side of the third stem;
the second polynucleotide comprises at least thirty two nucleotides but not more than eighty two nucleotides and comprises a secondary structure defined by: a second side of the third stem comprising five nucleotides wherein a bulge comprising three nucleotides is present between the third and fourth nucleotides of the second side of the third stem, a first side of a fourth stem comprising two nucleotides, a terminal loop comprising six nucleotides, a second side of the fourth stem comprising two nucleotides, a bulge comprising four nucleotides, a second side of the second stem comprising six nucleotides, and a dangling region comprising four nucleotides; and
the third polynucleotide comprises at least seven nucleotides but not more than fifty seven nucleotides and comprises a secondary structure defined by: a second side of the first stem comprising two nucleotides and a dangling region comprising five nucleotides.

110. The composition of claim 109 wherein the first polynucleotide comprises SEQ ID NO:159.

111. The composition of claim 109 wherein the second polynucleotide comprises SEQ ID NO:160.

112. The composition of claim 109 wherein the third polynucleotide comprises 5′-gnnnuag-3′.

113. A composition comprising a first polynucleotide and a second polynucleotide wherein:
the first polynucleotide comprises from about ten nucleotides to about twenty seven nucleotides comprising a secondary structure defined by: a first side of a first stem comprising from about six nucleotides to about sixteen nucleotides, a bulge comprising from about one nucleotide to about three nucleotides, and a first side of a second stem comprising from about two nucleotides to about six nucleotides wherein a first side of an internal loop comprising from about one nucleotide to about two nucleotides is present in the first side of the second stem. The second polynucleotide comprises from about twenty six nucleotides to about sixty five nucleotides comprising a secondary structure defined by: a second side of the second stem comprising from about two nucleotides to about six nucleotides a second side of the internal loop comprising from about two nucleotides to about five nucleotides is present in the second side of the second stem, a bulge comprising from about one nucleotide to about two nucleotides, a first side of a third stem comprising from about three nucleotides to about seven nucleotides, a terminal loop comprising from about three nucleotides to about seven nucleotides, a second side of the third stem comprising from about three nucleotides to about seven nucleotides, a bulge comprising from about three nucleotides to about seven nucleotides, and a second side of the first stem comprising from about six nucleotides to about sixteen nucleotides wherein a bulge comprising from about one nucleotide to about three nucleotides is present in the second side of the first stem and wherein a bulge comprising from about one nucleotide to about three nucleotides is present in the second side of the first stem and wherein a bulge comprising from about one nucleotide to about two nucleotides is present in the second side of the first stem.

114. The composition of claim 113 wherein the first polynucleotide comprises at least eighteen nucleotides but not more than sixty eight nucleotides and comprises a secondary structure defined by: a first side of a first stem comprising eleven nucleotides, a bulge comprising two nucleotides, and a first side of a second stem comprising four nucleotides wherein a first side of an internal loop comprising one nucleotide is present between the second and third nucleotides of the first side of the second stem; and
the second polynucleotide comprises at least forty four nucleotides but not more than ninety four nucleotides and comprises a secondary structure defined by: a second side of the second stem comprising four nucleotides a second side of the internal loop comprising three nucleotides is present between the second and third nucleotides of the second side of the second stem, a bulge comprising one nucleotide, a first side of a third stem comprising five nucleotides, a terminal loop comprising five nucleotides, a second side of the third stem comprising five nucleotides, a bulge comprising five nucleotides, and a second side of the first stem comprising eleven nucleotides wherein a bulge comprising two nucleotides is present between the fifth and sixth nucleotides of the second side of the first stem and wherein a bulge comprising two nucleotides is present between the sixth and seventh nucleotides of the second side of the first stem and wherein a bulge comprising one nucleotide is present between the tenth and eleventh nucleotides of the second side of the first stem.

115. The composition of claim 114 wherein the first polynucleotide comprises SEQ ID NO:161.

116. The composition of claim 114 wherein the second polynucleotide comprises SEQ ID NO:162.

117. A polynucleotide comprising from about fifteen nucleotides to about ninety nucleotides comprising a secondary structure defined by: a first side of a stem comprising from about five nucleotides to about thirteen nucleotides wherein a first side of an internal loop comprising from about two nucleotides to about five nucleotides is present in the first side of the stem, a terminal loop comprising from about two nucleotides to about six nucleotides, and a second side of the first stem comprising from about five nucleotides to about thirteen nucleotides wherein a second side of the internal loop comprising from about one nucleotide to about three nucleotides is present in the second side of the stem.

118. The polynucleotide of claim 117 comprising at least twenty seven nucleotides and up to seventy seven nucleotides comprising a secondary structure defined by: a first side of a stem comprising nine nucleotides wherein a first side of an internal loop comprising three nucleotides is present between the sixth and seventh nucleotides of the first side of the stem, a terminal loop comprising four nucleotides, and a second side of the first stem comprising nine nucleotides wherein a second side of the internal loop comprising two nucleotides is present between the third and fourth nucleotides of the second side of the stem.

119. The polynucleotide of claim 118 comprising SEQ ID NO:163.

120. A composition comprising a first polynucleotide and a second polynucleotide wherein:
the first polynucleotide comprises from about four nucleotides to about twelve nucleotides comprising a secondary structure defined by: a first side of a first stem comprising from about two nucleotides to about six nucleotides and a first side of a second stem comprising from about two nucleotides to about six nucleotides;
the second polynucleotide comprises from about seven nucleotides to about nineteen nucleotides comprising a secondary structure defined by: a second side of the second stem comprising from about two nucleotides to about six nucleotides, a bulge comprising from about two nucleotides to about six nucleotides, and a first side of a third stem comprising from about three nucleotides to about seven nucleotides; and
the third polynucleotide comprises from about eight nucleotides to about twenty nucleotides comprising a secondary structure defined by: a second side of the third stem comprising from about three nucleotides to about seven nucleotides wherein a bulge comprising from about one nucleotide to about two nucleotides is present in the second side of the third stem, a bulge comprising from about two nucleotides to about five nucleotides, and second side of the first stem comprising from about two nucleotides to about six nucleotides.

121. The composition of claim 120 wherein the first polynucleotide comprises at least eight nucleotides but not more than fifty eight nucleotides and comprises a secondary structure defined by: a first side of a first stem comprising four nucleotides and a first side of a second stem comprising four eight nucleotides;
the second polynucleotide comprises at least thirteen nucleotides but not more than sixty three nucleotides and comprises a secondary structure defined by: a second side of the second stem comprising four nucleotides, a bulge comprising four nucleotides, and a first side of a third stem comprising five nucleotides; and
the third polynucleotide comprises at least thirteen nucleotides but not more than sixty three nucleotides and comprises a secondary structure defined by: a second side of the third stem comprising five nucleotides wherein a bulge comprising one nucleotide is present between the second and third nucleotides of the second side of the third stem, a bulge comprising three nucleotides, and second side of the first stem comprising four nucleotides.

122. The composition of claim 121 wherein the first polynucleotide comprises 5′-gnnnnnnn-3′.

123. The composition of claim 121 wherein the second polynucleotide comprises SEQ ID NO:164.

124. The composition of claim 121 wherein the third polynucleotide comprises SEQ ID NO:165.

125. A composition comprising a first polynucleotide and a second polynucleotide wherein:
the first polynucleotide comprises from about five nucleotides to about fifteen nucleotides comprising a secondary structure defined by: a first side of a stem comprising from about three nucleotides to about nine nucleotides wherein a first side of an internal loop comprising from about one nucleotide to about three nucleotides is present between the second and third nucleotides of the first side of the stem and wherein a bulge comprising from about one nucleotide to about three nucleotides is present between the third and fourth nucleotides of the first side of the stem; and
the second polynucleotide comprises from about five nucleotides to about fifteen nucleotides comprising a secondary structure defined by: a second side of the stem comprising from about three nucleotides to about nine nucleotides wherein a second side of the internal loop comprising from about two nucleotides to about six nucleotides is present in the second side of the stem.

126. The composition of claim 125 wherein the first polynucleotide comprises at least ten nucleotides but not more than sixty nucleotides and comprises a secondary structure defined by: a first side of a stem comprising six nucleotides wherein a first side of an internal loop comprising two nucleotides is present between the second and third nucleotides of the first side of the stem and wherein a bulge comprising two nucleotides is present between the third and fourth nucleotides of the first side of the stem; and
the second polynucleotide comprises at least nine nucleotides but not more than fifty nine nucleotides and comprises a secondary structure defined by: a second side of the stem comprising six nucleotides wherein a second side of the internal loop comprising four nucleotides is present between the fourth and fifth nucleotides of the second side of the stem.

127. The composition of claim 126 wherein the first polynucleotide comprises SEQ ID NO:166.

128. The composition of claim 126 wherein the second polynucleotide comprises 5′-nnunnagnn-3′.
